SEQUENCE LISTING (66 sequences)

SEQ ID NO: 1 - protein, 437 aa, Homo sapiens
MSWSLHPRNLILYFYALLFLSSTCVAYVATRDNCCILDERFGSYCPTTCGIADFLSTYQTKVDKDLQSLEDILHQVENKTSEVKQLIKAIQLTYNPDESSKPNMIDAATLKSRKMLEEIMKYEASILTHDSSIRYLQEIYNSNNQKIVNLKEKVAQLEAQCQEPCKDTVQIHDITGKDCQDIANKGAKQSGLYFIKPLKANQQFLVYCEIDGSGNGWTVFQKRLDGSVDFKKNWIQYKEGFGHLSPTGTTEFWLGNEKIHLISTQSAIPYALRVELEDWNGRTSTADYAMFKVGPEADKYRLTYAYFAGGDAGDAFDGFDFGDDPSDKFFTSHNGMQFSTWDNDNDKFEGNCAEQDGSGWWMNKCHAGHLNGVYYQGGTYSKASTPNGYDNGIIWATWKTRWYSMKKTTMKIIPFNRLTIGEGQQHHLGGAKQAGDV

SEQ ID NO: 2 - protein, 17 aa, Artificial Sequence
  A synthetic sequence
CKKTTMKIIPFNRLTIG

SEQ ID NO: 3 - protein, 443 aa, Mus musculus
MSWSLQPPSFLLCCLLLLFSPTGLAYVATRDNCCILDERFGSFCPTTCGIADFLSSYQTDVDNDLRTLEDILFRAENRTTEAKELIKAIQVYYNPDQPPKPGMIDSATQKSKKMVEEIVKYEALLLTHETSIRYLQEIYNSNNQKITNLKQKVAQLEAQCQEPCKDSVQIHDTTGKDCQEIANKGAKESGLYFIRPLKAKQQFLVYCEIDGSGNGWTVLQKRIDGSLDFKKNWIQYKEGFGHLSPTGTTEFWLGNEKIHLISMQSTIPYALRIQLKDWNGRTSTADYAMFRVGPESDKYRLTYAYFIGGDAGDAFDGYDFGDDPSDKFFTSHNGMQFSTWDNDNDKFEGNCAEQDGSGWWMNKCHAGHLNGVYHQGGTYSKSSTTNGFDDGIIWATWKSRWYSMKETTMKIIPFNRLSIGEGQQHHMGGSKQVSVDHEVEIEY

SEQ ID NO: 4 - protein, 14 aa, Artificial Sequence
  A synthetic sequence
CGWTVLQKRIDGSL

SEQ ID NO: 5 - protein, 158 aa, Mus musculus
TFDSPYQVRRMRFSAQLLGLLVLWIPGSTADIVMTQAAFSNPITLGTSASMSCRSSKSLLHSSGITYLSWYLQKPGQSPQLLIYQMSNLASGVPDRFSSSGSGTDFTLRISRVEAEDVGVYYCAQNLELPLTFGAGTKLELKRADAAPTVSACTKGEF

SEQ ID NO: 6 - protein, 16 aa, Mus musculus
RSSKSLLHSSGITYLS

SEQ ID NO: 7 - protein, 7 aa, Mus musculus
QMSNLAS

SEQ ID NO: 8 - protein, 9 aa, Mus musculus
AQNLELPLT

SEQ ID NO: 9 - protein, 166 aa, Mus musculus
NTAFAGFGRNMRSLFSLQLLSTQDLAMGWSCIIVLLVSTATGVHSQVQLQQPGAELVRPGTSVKLSCKASGYTFTSYWIHWVKQRPGQGLEWIGLIDPSDSYTNYNQKFRGKATLTVDTSSSTAYMQLSSLTSEDSAVYYCASSDPTGCWGQGTTLTVSPASTTPP

SEQ ID NO: 10 - protein, 10 aa, Mus musculus
GYTFTSYWIH

SEQ ID NO: 11 - protein, 16 aa, Mus musculus
LIDPSDSYTNYNQKFR

SEQ ID NO: 12 - protein, 6 aa, Mus musculus
SDPTGC

SEQ ID NO: 13 - DNA, 474 bp, Mus musculus
acttttgactcaccatatcaagttcgcagaatgaggttctctgctcagcttctggggctgcttgtgctctggatccctggatccactgcagatattgtgatgacgcaggctgcattctccaatccaatcactcttggaacatcagcttccatgtcctgcaggtctagtaagagtctcctacatagtagtggcatcacttatttgtcttggtatctgcagaagccaggccagtctcctcagctcctgatttatcagatgtccaaccttgcctcaggagtcccagacaggttcagtagcagtgggtcaggaactgatttcacactgagaattagccgagtggaggctgaggatgtgggtgtttattactgtgctcaaaatctagaacttccgctcacgttcggtgctgggaccaagctggagctgaaacgggctgatgctgcaccaactgtatccgcatgcaccaagggcgaattc

SEQ ID NO: 14 - DNA, 499 bp, Mus musculus
gaacactgcgtttgctggctttggaagaaacatgagatcactgttctctctacagttactgagcacacaggacctcgccatgggatggagctgtatcattgtcctcttggtatcaacagctacaggtgtccactcccaggtccaactgcagcagcctggggctgagctggtgaggcctgggacttcagtgaagttgtcctgcaaggcttctggctacaccttcaccagctactggatacactgggtaaagcagaggcctggacaaggccttgagtggatcggactgattgatccttctgatagttatactaactacaatcaaaagttcaggggcaaggccacattgactgtagacacatcctccagcacagcctacatgcagctcagcagcctgacatctgaggactctgcggtctattactgtgcaagctccgatcctacaggctgctggggccaaggcaccactctcacagtctccccagctagcacaacaccccca

SEQ ID NO: 15 - DNA, 48 bp, Mus musculus
aggtctagtaagagtctcctacatagtagtggcatcacttatttgtct

SEQ ID NO: 16 - DNA, 20 bp, Mus musculus
cagatgtccaaccttgcctc

SEQ ID NO: 17 - DNA, 27 bp, Mus musculus
gctcaaaatctagaacttccgctcacg

SEQ ID NO: 18 - DNA, 30 bp, Mus musculus
ggctacaccttcaccagctactggatacac

SEQ ID NO: 19 - DNA, 51 bp, Mus musculus
ctgattgatccttctgatagttatactaactacaatcaaaagttcaggggc

SEQ ID NO: 20 - DNA, 18 bp, Mus musculus
tccgatcctacaggctgc

SEQ ID NO: 21-29 - skipped (9 entries)

SEQ ID NO: 30 - protein, 1273 aa, SARS CoV
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 31 - protein, 254 aa, Corona Virus
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLE

SEQ ID NO: 32 - protein, 610 aa, Corona Virus
CDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLH

SEQ ID NO: 33 - protein, 1255 aa, Corona Virus
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 34 - protein, 191 aa, SARS CoV
TNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFEGSLEVLFQ

SEQ ID NO: 35 - protein, 685 aa, Artificial Sequence
  A synthetic sequence
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 36 - protein, 541 aa, Artificial Sequence
  A synthetic sequence
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 37 - protein, 318 aa, Artificial Sequence
  A synthetic sequence
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNF

SEQ ID NO: 38 - protein, 221 aa, Artificial Sequence
  A synthetic sequence
QPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 39 - protein, 367 aa, Artificial Sequence
  A synthetic sequence
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAR

SEQ ID NO: 40 - protein, 588 aa, Artificial Sequence
  A synthetic sequence
SVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 41 - protein, 11 aa, Homo sapiens
YLLKDLWQKRQ

SEQ ID NO: 42 - protein, 19 aa, Homo sapiens
QSGLYFIKPLKANQQFLVY

SEQ ID NO: 43 - protein, 32 aa, Homo sapiens
DNGIIWATWKTRWYSMKKTTMKIIPFNRLTIG

SEQ ID NO: 44 - protein, 19 aa, Artificial Sequence
  A synthetic sequence
YSMKKTTMKIIPFNRLTIG

SEQ ID NO: 45 - protein, 19 aa, Artificial Sequence
  A synthetic sequence
ASMKKTTMKIIPFNRLTIG

SEQ ID NO: 46 - protein, 19 aa, Artificial Sequence
  A synthetic sequence
YAMKKTTMKIIPFNRLTIG

SEQ ID NO: 47 - protein, 19 aa, Artificial Sequence
  A synthetic sequence
YSAKKTTMKIIPFNRLTIG

SEQ ID NO: 48 - protein, 19 aa, Artificial Sequence
  A synthetic sequence
YSMAKTTMKIIPFNRLTIG

SEQ ID NO: 49 - protein, 19 aa, Artificial Sequence
  A synthetic sequence
YSMKATTMKIIPFNRLTIG

SEQ ID NO: 50 - protein, 19 aa, Artificial Sequence
  A synthetic sequence
YSMKKATMKIIPFNRLTIG

SEQ ID NO: 51 - protein, 19 aa, Artificial Sequence
  A synthetic sequence
YSMKKTAMKIIPFNRLTIG

SEQ ID NO: 52 - protein, 19 aa, Artificial Sequence
  A synthetic sequence
YSMKKTTAKIIPFNRLTIG

SEQ ID NO: 53 - protein, 19 aa, Artificial Sequence
  A synthetic sequence
YSMKKTTMAIIPFNRLTIG

SEQ ID NO: 54 - protein, 19 aa, Artificial Sequence
  A synthetic sequence
YSMKKTTMKAIPFNRLTIG

SEQ ID NO: 55 - protein, 19 aa, Artificial Sequence
  A synthetic sequence
YSMKKTTMKIAPFNRLTIG

SEQ ID NO: 56 - protein, 19 aa, Artificial Sequence
  A synthetic sequence
YSMKKTTMKIIAFNRLTIG

SEQ ID NO: 57 - protein, 19 aa, Artificial Sequence
  A synthetic sequence
YSMKKTTMKIIPANRLTIG

SEQ ID NO: 58 - protein, 19 aa, Artificial Sequence
  A synthetic sequence
YSMKKTTMKIIPFARLTIG

SEQ ID NO: 59 - protein, 19 aa, Artificial Sequence
  A synthetic sequence
YSMKKTTMKIIPFNALTIG

SEQ ID NO: 60 - protein, 19 aa, Artificial Sequence
  A synthetic sequence
YSMKKTTMKIIPFNRATIG

SEQ ID NO: 61 - protein, 19 aa, Artificial Sequence
  A synthetic sequence
YSMKKTTMKIIPFNRLAIG

SEQ ID NO: 62 - protein, 19 aa, Artificial Sequence
  A synthetic sequence
YSMKKTTMKIIPFNRLTAG

SEQ ID NO: 63 - protein, 19 aa, Artificial Sequence
  A synthetic sequence
YSMKKTTMKIIPFNRLTIA

SEQ ID NO: 64 - protein, 9 aa, Artificial Sequence
  A synthetic sequence
  VARIANT (1)...(9): Xaa = Any Amino Acid
IIPFXRLXI

SEQ ID NO: 65 - protein, 1152 aa, Homo sapiens
MALRVLLLTALTLCHGFNLDTENAMTFQENARGFGQSVVQLQGSRVVVGAPQEIVAANQRGSLYQCDYSTGSCEPIRLQVPVEAVNMSLGLSLAATTSPPQLLACGPTVHQTCSENTYVKGLCFLFGSNLRQQPQKFPEALRGCPQEDSDIAFLIDGSGSIIPHDFRRMKEFVSTVMEQLKKSKTLFSLMQYSEEFRIHFTFKEFQNNPNPRSLVKPITQLLGRTHTATGIRKVVRELFNITNGARKNAFKILVVITDGEKFGDPLGYEDVIPEADREGVIRYVIGVGDAFRSEKSRQELNTIASKPPRDHVFQVNNFEALKTIQNQLREKIFAIEGTQTGSSSSFEHEMSQEGFSAAITSNGPLLSTVGSYDWAGGVFLYTSKEKSTFINMTRVDSDMNDAYLGYAAAIILRNRVQSLVLGAPRYQHIGLVAMFRQNTGMWESNANVKGTQIGAYFGASLCSVDVDSNGSTDLVLIGAPHYYEQTRGGQVSVCPLPRGRARWQCDAVLYGEQGQPWGRFGAALTVLGDVNGDKLTDVAIGAPGEEDNRGAVYLFHGTSGSGISPSHSQRIAGSKLSPRLQYFGQSLSGGQDLTMDGLVDLTVGAQGHVLLLRSQPVLRVKAIMEFNPREVARNVFECNDQVVKGKEAGEVRVCLHVQKSTRDRLREGQIQSVVTYDLALDSGRPHSRAVFNETKNSTRRQTQVLGLTQTCETLKLQLPNCIEDPVSPIVLRLNFSLVGTPLSAFGNLRPVLAEDAQRLFTALFPFEKNCGNDNICQDDLSITFSFMSLDCLVVGGPREFNVTVTVRNDGEDSYRTQVTFFFPLDLSYRKVSTLQNQRSQRSWRLACESASSTEVSGALKSTSCSINHPIFPENSEVTFNITFDVDSKASLGNKLLLKANVTSENNMPRTNKTEFQLELPVKYAVYMVVTSHGVSTKYLNFTASENTSRVMQHQYQVSNLGQRSLPISLVFLVPVRLNQTVIWDRPQVTFSENLSSTCHTKERLPSHSDFLAELRKAPVVNCSIAVCQRIQCDIPFFGIQEEFNATLKGNLSFDWYIKTSHNHLLIVSTAEILFNDSVFTLLPGQGAFVRSQTETKVEPFEVPNPLPLIVGSSVGGLLLLALITAALYKLGFFKRQYKDMMSEGGPPGAEPQ

SEQ ID NO: 66 - protein, 8 aa, Artificial Sequence
  A synthetic sequence
AGHHHHHH

(19) United States
(12) Patent Application Publication
(10) Pub. No.: US 2024/0059761 A1
(43) Pub. Date: Feb. 22, 2024

(54) Use of fibrin-targeting immunotherapy to reduce Coronavirus pathogenesis

(71) Applicant: The J. David Gladstone Institutes, a testamentary trust established under the Will of J. David, San Francisco, CA (US)

(72) Inventors: Katerina Akassoglou, San Francisco, CA (US); Jae Kyu Ryu, San Francisco, CA (US); Warner C. Greene, San Francisco, CA (US); Mauricio Montano, San Francisco, CA (US)

(21) Appl. No.: 18/267,710

(22) PCT Filed: Dec. 16, 2021
(86) PCT No.: PCT/US2021/063705

§ 371 Date: Jun. 15, 2023

Related U.S. Application Data
(60) Provisional application No. 63/126,030, filed on Dec. 16, 2020.

(51) Int. Cl. C07K 16/10; C07K 16/36; A61P 29/00; A61P 37/06; A61P 31/14

(52) U.S. Cl. CPC C07K 16/1003; C07K 16/36; A61P 29/00; A61P 37/06; A61P 31/14; A61K 2039/507

(57) ABSTRACT

As described herein, anti-fibrin antibodies can reduce and treat the symptoms of Coronavirus infection, including CoV-ID-19 infection. Compositions and methods are described herein that include active agents such as anti-fibrin antibodies that can inhibit inflammation in the lung and other tissues. These methods and compositions can inhibit the binding of SARS-COV-2 or SARS-COV-1 spike protein to fibrin.

### CROSS REFERENCE TO RELATED APPLICATIONS

This application claims the benefit of priority to U.S. Provisional Patent Application Ser. No. 63/126,030, filed Dec. 16, 2020, the complete disclosure of which is incorporated herein by reference in its entirety.

### INCORPORATION BY REFERENCE OF SEQUENCE LISTING PROVIDED AS A TEXT FILE

A Sequence Listing is provided herewith as a text file, “2199599.txt” created on Dec. 15, 2021 and having a size of 77,824 bytes. The contents of the text file are incorporated by reference herein in their entirety.

### BACKGROUND

SARS-COV-2 is highly infectious coronavirus that caused a global pandemic beginning in 2019 (COVID-19). Highly effective and safe RNA and adenoviral vaccines have been developed, but vaccine hesitancy, lack of vaccine access in the developing world, and the repeated emergence of viral variants displaying increased infectivity and/or immuno-evasive properties has left hundreds of millions of people around the globe vulnerable to the debilitating and lethal effects of this virus.

Thrombosis and inflammation are hallmarks of acute coronavirus infection. Effective antithrombotic therapy has been difficult to achieve in part due to diminished clot breakdown. Glucocorticoids are partially effective in blunting the host inflammatory response that ultimately drives the lethal effects of viral infection. Even when infected individuals ward off the initial viral illness, they remain vulnerable to Long COVID or Post-Acute Sequelae of COVID-19 (PASC) that can involve multiple organs including the lung, heart, brain, and joints. No effective therapies have been identified for Long COVID although multiple reports suggest that Spike-based vaccinations are beneficial. Without question, more effective therapeutic approaches to manage both acute COVID-19 and Long COVID are urgently needed.

### SUMMARY

Described herein are compositions and methods for treating coronavirus (e.g., SARS-COV-2 and/or SARS-COV-1) infection. As illustrated herein, the spike protein of SARS-COV-2 binds fibrinogen and fibrin, and antibodies directed against fibrin are surprisingly effective at reducing such binding as well as reducing the inflammation associated with coronavirus infection. Fibrin is deposited in tissues of patients infected with SARS-COV-2 including in the brain, gut, kidneys, vascular system, and lungs. Such fibrin deposition may contribute to the short-term and long-term symptoms of SARS-COV-2 infection. No current therapeutics prevent the fibrin-mediated effects that can induce inflammation and thrombosis caused by coronaviruses, including SARS-COV-2.

The compositions provided herein include antibodies, small molecules, and polypeptides that can bind to fibrinogen and fibrin that can reduce the adverse effects of Coronavirus, including SARS-COV-2 and/or SARS-COV-1 infection. The compositions can also include anti-Spike protein antibodies, for example, anti-Spike protein antibodies that reduce Spike protein binding to fibrinogen or fibrin. Any of the antibodies, small molecules, and polypeptides can inhibit coronavirus virion and coronavirus spike protein binding to fibrinogen and fibrin. The compositions can include human or humanized anti-fibrin or anti-fibrinogen pr anti-Spike protein antibodies. Such antibodies can, for example, bind to a fibrin/fibrinogen epitope with one or more of the following sequences:

SEQ ID NO: 2
Bβ₁₁₉₋₁₂₉ (YLLKDLWQKRQ, SEQ ID NO: 41),,
γ₁₆₃₋₁₈₁ (QSGLYFIKPLKANQQFLVY; SEQ ID NO: 42),
γ₃₆₄₋₃₉₅ (DNGIIWATWKTRWYSMKKTTMKIIPFNRLTIG; SEQ ID NO: 43),
or
(SEQ ID NO: 64)
IIPFXRLXI.

In some cases, the antibodies can have a CDR region with a sequence that includes SEQ ID NO:6-8, 10, 11 or 12, or combination of CDR regions with sequences that include SEQ ID NO:6-8, 10, 11, and 12. Other agents that bind fibrin/fibrinogen can also be used that block the interaction between Coronavirus Spike protein and fibrin/fibrinogen, including anti-Spike protein antibodies. The compositions can include agents such as antibodies, small molecules, and polypeptides in an amount sufficient to reduce the interaction between Coronavirus Spike protein and fibrin/fibrinogen. Such agents can also reduce the adverse effects and symptoms of Coronavirus infection. For example, the compositions can include the antibodies, small molecules, and polypeptides in an amount sufficient to reduce inflammation in at least one of the brain, gut, kidneys, vascular system, or lungs.

Hence, the compositions can include the antibodies, small molecules, and polypeptides in an amount sufficient to reduce Coronavirus virus binding to fibrin or fibrinogen, that can reduce Coronavirus spike protein binding to fibrin or fibrinogen, that can reduce Mac-1 binding to fibrin or fibrinogen, or a combination thereof.

Also described herein are methods that involve administering a composition that includes antibodies, small molecules, and polypeptides to a subject infected with Coronavirus, where the antibodies, small molecules, and polypeptides can bind to fibrin, the Coronavirus spike protein, or a combination thereof. In some cases the composition can include anti-fibrin antibodies alone. In some cases the compositions can include a combination of anti-fibrin antibodies with other agents, including anti-Spike protein antibodies. Such methods can reduce the short-term and long-term symptoms of Coronavirus infection. For example, the methods can reduce inflammation in at least one of the brain, gut, kidneys, vascular system, or lungs. Such methods can reduce Coronavirus virus binding to fibrin or fibrinogen. Such methods can reduce Coronavirus spike protein binding to fibrin or fibrinogen.

The antibodies used in the compositions and methods can be human antibodies or humanized antibodies. For example, the antibodies can bind to at least one epitope

SEQ ID NO: 2,
Bβ₁₁₉₋₁₂₉ (YLLKDLWQKRQ, SEQ ID NO: 41),
γ₁₆₃₋₁₈₁ (QSGLYFIKPLKANQQFLVY; SEQ ID NO: 42),
γ₃₆₄₋₃₉₅ (DNGIIWATWKTRWYSMKKTTMKIIPFNRLTIG;
SEQ ID NO: 43),
IIPFXRLXI (SEQ ID NO: 64),

or a combination thereof. In some cases, the antibodies can have one or more CDR regions with a sequence that has SEQ ID NO:6-8, 10-12. In some cases, the antibodies can have a combination of CDR regions with sequences that include SEQ ID NO:6-8, 10, 11, and 12.

The compositions and methods described herein can reduce inflammation, oxidative stress, fibrin deposition, or a combination thereof, in tissues of a subject. The compositions and methods described herein can inhibit at least 50% of SARS-CoV-2 spike protein, SARS-COV-1 spike protein, SARS-COV-2 viral particle, SARS-CoV-1 viral particle, or Mac-1 binding to the fibrin or fibrinogen, compared to SARS-CoV-2 spike protein, SARS-COV-1 spike protein, SARS-COV-2 viral particle, SARS-CoV-1 viral particle, or Mac-1 binding to fibrin or fibrinogen in a control subject who did not receive the composition.

### DESCRIPTION OF THE FIGURES

FIGS. 1A-1B illustrate that administration of the anti-fibrin SB8 antibodies reduces inflammation in the lungs of SARS-COV-2 infected mice compared to the inflammation observed for control SARS-COV-2 infected mice which had received non-reactive IgG2b antibodies. FIG. 1A shows images of lung sections stained with anti-macrophage antibodies that were obtained from SARS-COV-2 infected mice treated with anti-fibrin 5B8 antibodies (right) or with SARS-COV-2 infected mice treated with non-reactive IgG2b antibodies (control, left). FIG. 1B graphically illustrates that the number of Mac-2⁺ macrophages was significantly reduced in the lungs of mice that received the 5B8 antibodies after SARS-COV-2 infection, compared to the control mice that were infected with after SARS-COV-2 but that had received the non-reactive IgG2b antibodies

FIGS. 2A-2B graphically illustrate that fibrin and fibrinogen bind to the SARS-CoV-2 spike protein. FIG. 2A graphically illustrates that fibrinogen binds to the SARS-COV-2 spike protein. FIG. 2B graphically illustrates that fibrin binds to the SARS-COV-2 spike protein.

FIG. 3 graphically illustrates that 5B8 anti-fibrin antibodies inhibit binding of the SARS-COV-2 spike protein to fibrin. Varying amounts of 5B8 anti-fibrin antibodies were incubated in solution with a set amount of SARS-COV-2 spike protein on the fibrin-coated plates, and the amount of SARS-COV-2 spike protein bound to the fibrin-coated plates was detected with a labeled anti-spike protein antibody. As illustrated when 5B8 anti-fibrin antibodies were incubated in solution with the SARS-CoV-2 spike protein, less SARS-COV-2 spike protein binds to the immobilized fibrin.

FIGS. 4A-4H illustrate the interaction between SARS-COV-2 Spike and fibrinogen. FIG. 4A graphically illustrates fibrin polymerization in healthy human donor plasma in the presence or absence of Spike protein as measured by turbidity assays. Data are representative of four independent experiments with similar results. As illustrated, significantly more fibrin polymerization occurred when SARS-COV-2 Spike protein is present than when it is not. FIG. 4B shows scanning electron microscopic (SEM) images of fibrin clots in healthy human donor plasma in the presence and absence of Spike protein. Scale bar, 1 μm. Topographic visualization of fibrin fiber surface is provided for the SEM images. Also shown is a graph that shows the quantified fibrin fiber radius and intersection density in the presence and absence of SARS-COV-2 Spike protein. Data are from three independent experiments (mean±s.e.m.). ****/<0.0001, *** P<0.001 (multiple testing Holm procedure and two-tailed Mann-Whitney test). FIG. 4C graphically illustrates binding of recombinant SARS-COV-2 Spike protein (Spike) to fibrinogen or fibrin as detected by ELISA using the absorbance at 450 nm (A450), plus the dissociation constants (Kd). Representative binding curvefits are shown from two independent experiments performed in duplicates (mean±s.e.m.). FIG. 4D shows blots of fibrinogen immunoprecipitated (IP) with His-tagged recombinant trimeric SARS-COV-2 Spike protein produced in CHO cells (left) or monomeric SARS-COV-2 Spike produced in E.coli (right) blotted with anti-spike, anti-His or anti-fibrinogen. Representative immunoblots from three independent experiments are shown. FIG. 4E shows beatmaps of Spike protein binding sites on fibrinogen chains Aα, Bβ, and γ. Peptide array mapping was performed with immobilized peptides of fibrinogen chains Aα, Bβ, and γ blotted with Spike protein. The heatmap shows the signal intensity of binding sites (red-orange in the original) indicated by shading above the amino acid sequence locations on the fibrinogen chains. As shown, binding occurred within the

Bβ₁₁₉₋₁₂₉ (YLLKDLWQKRQ, SEQ ID NO: 41),
γ₁₆₃₋₁₈₁ (QSGLYFIKPLKANQQFLVY; SEQ ID NO: 42)
and
γ₃₆₄₋₃₉₅ (DNGIIWATWKTRWYSMKKTTMKIIPFNRLTIG;
SEQ ID NO: 43)

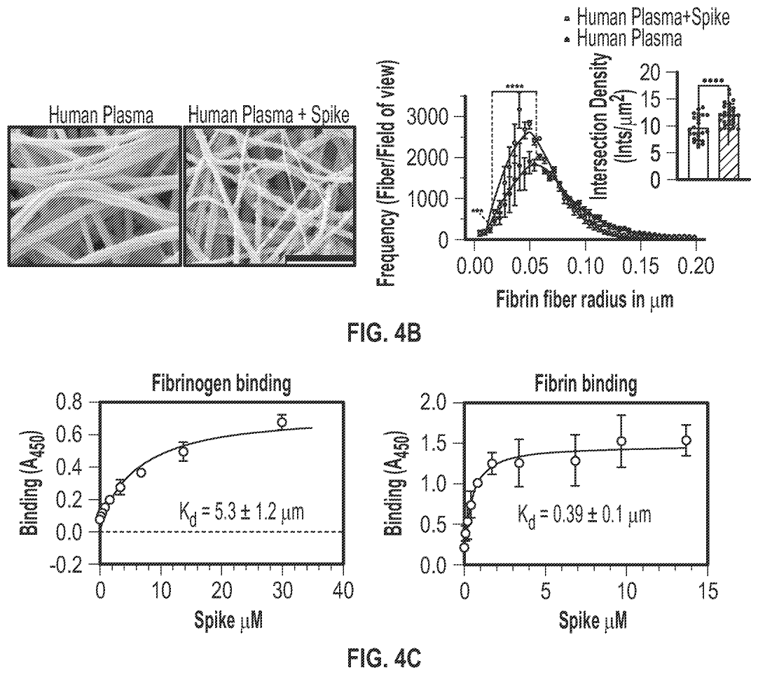

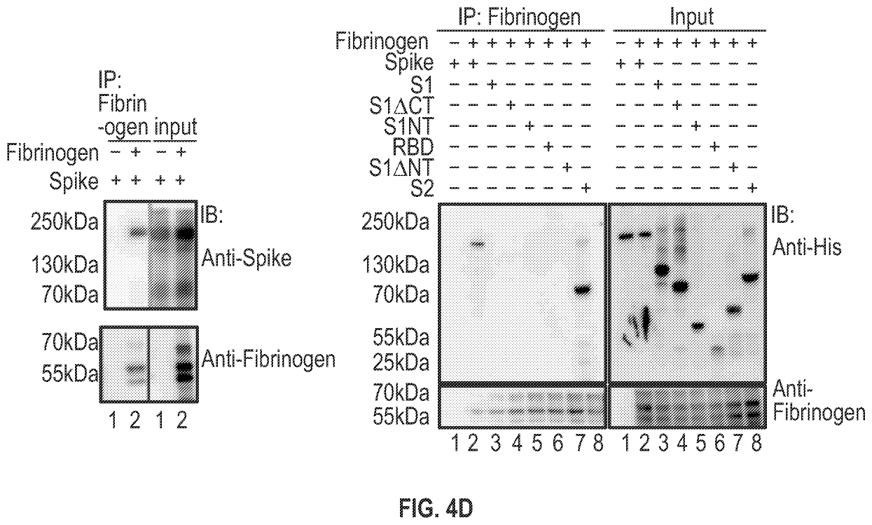

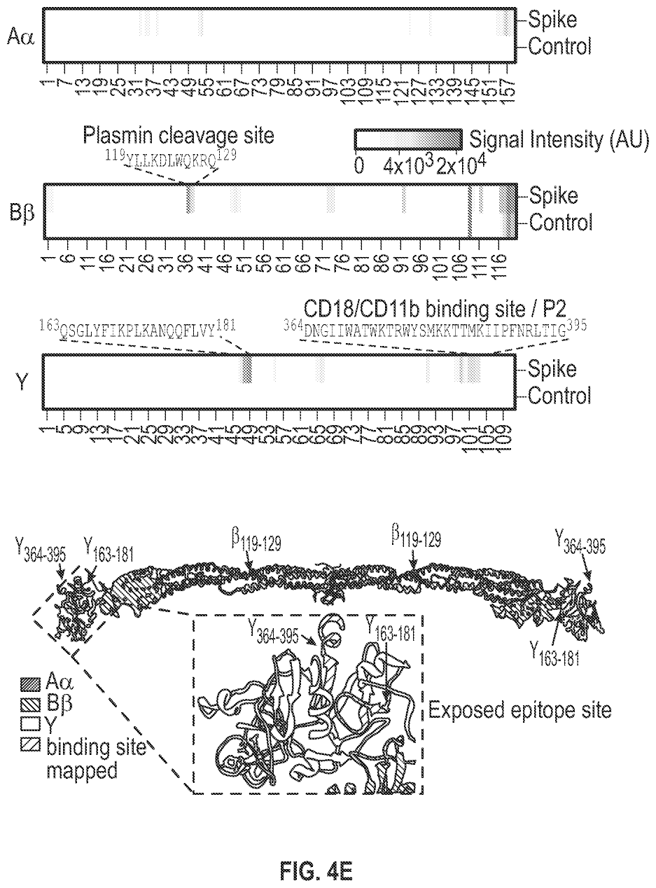

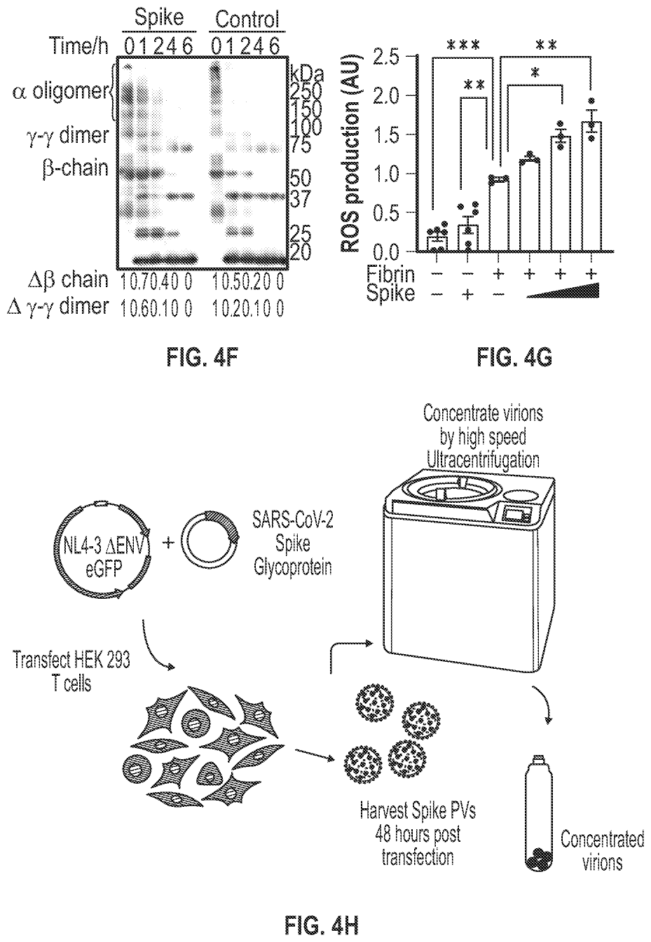

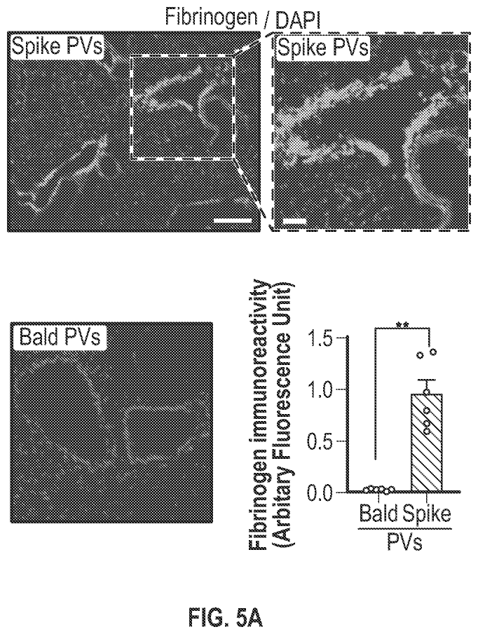

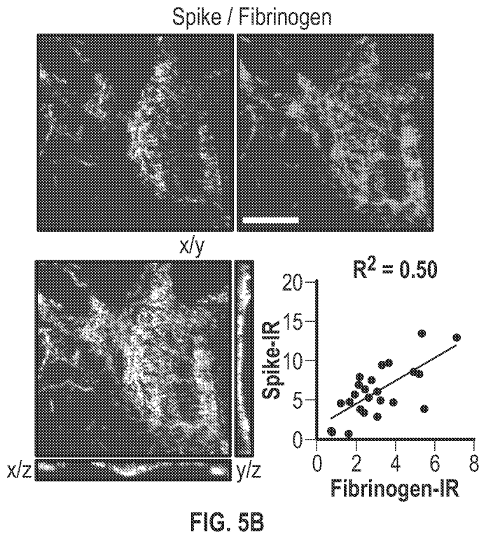

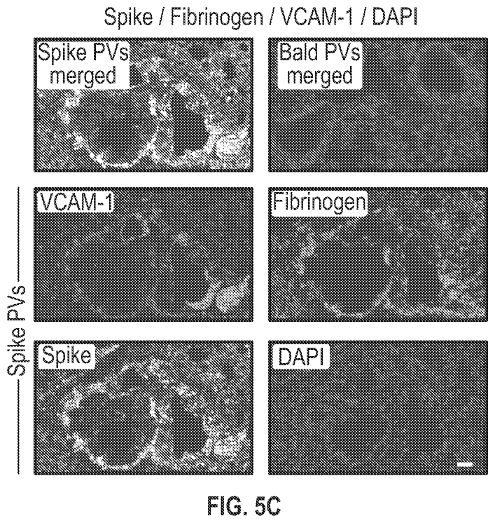

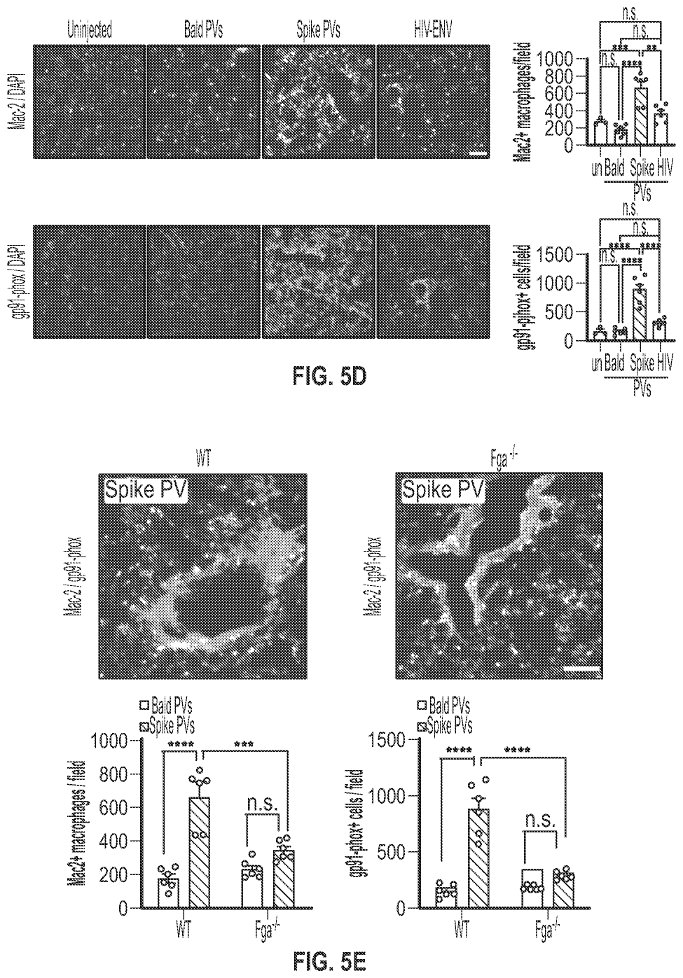

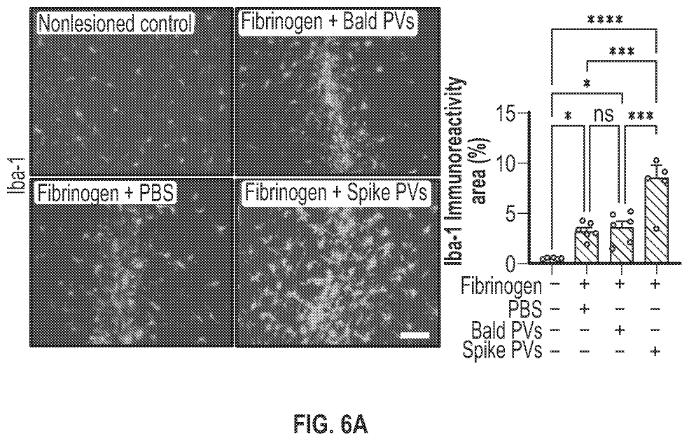

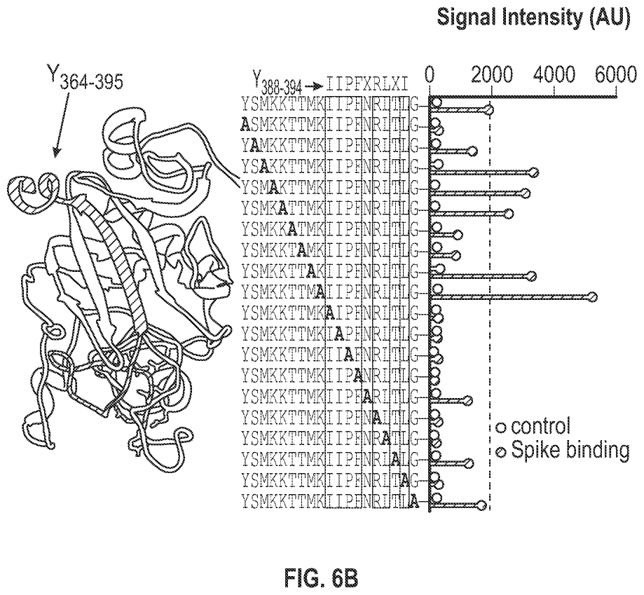

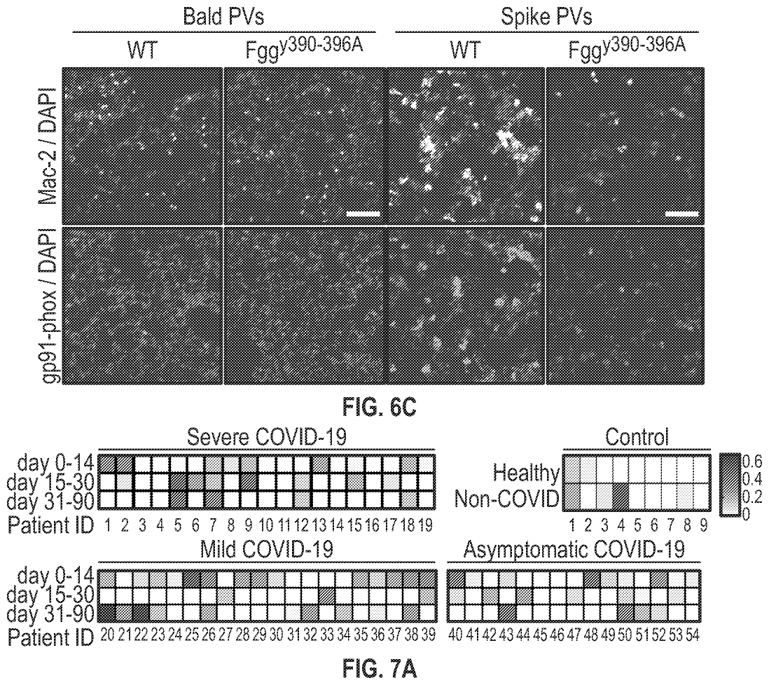

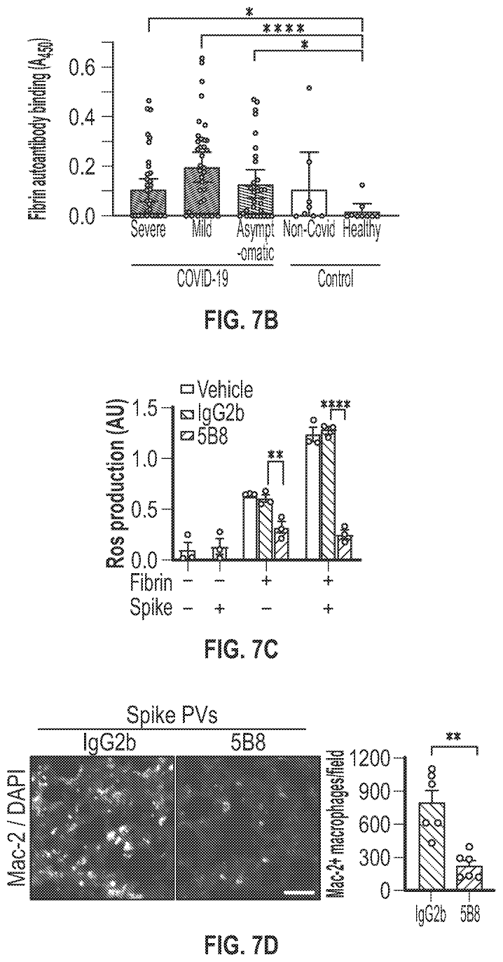

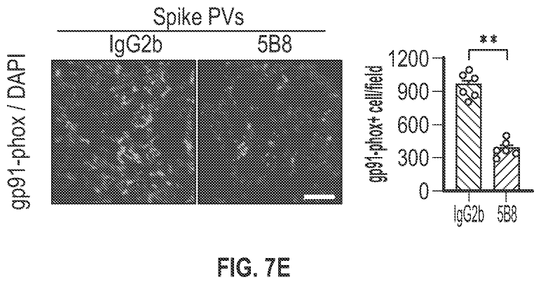

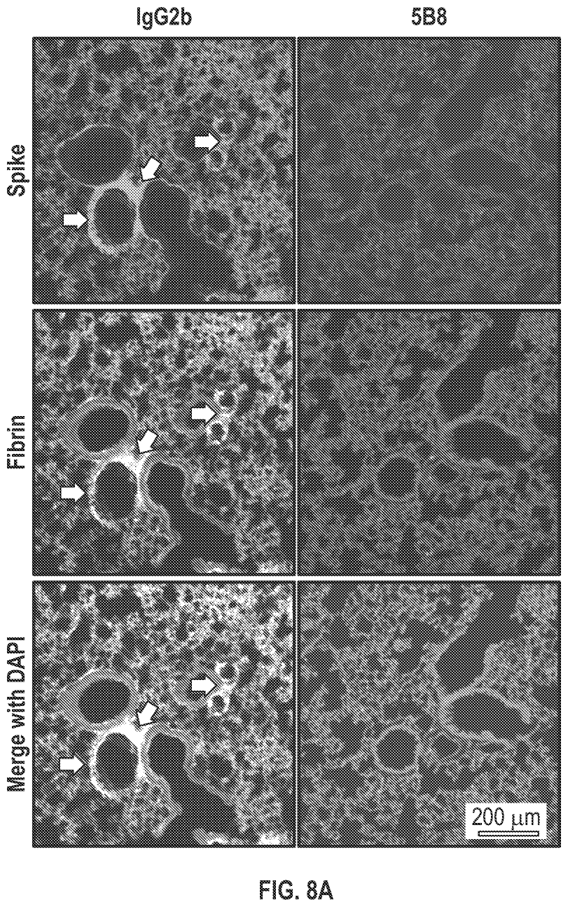

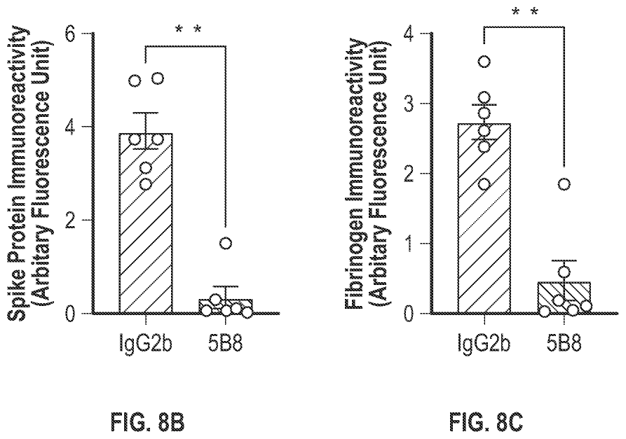

sites of fibrinogen. The key indicates fluorescence intensities signal values from low (white) to high (grey to darker grey). The crystal structure of fibrinogen (PDB:3GHG) is illustrated below where the three peptides β119-129, γ163-181 and γ364-395 are highlighted by shading (red in the original). The structural proximity of the γ163-181 and γ364-395 peptides indicates that they may form a 3D conformational epitope (inset). FIG. 4F shows an immunoblot of fibrin degradation after 0, 1, 2, 4 and 6 h of plasmin digestion in the presence or absence of SARS-COV-2 Spike protein. Data are from five (timepoints at 0, 2 hours and 4 hours) or three (timepoints at 1 hour and 6 hours) independent experiments (mean±s.e.m.). A representative immunoblot is shown. FIG. 4G graphically illustrates quantification of reactive oxygen species (ROS) production detected with dihydroethidum in unstimulated bone marrow-derived macrophages (BMDMs) or BMDMs stimulated for 24 hours with fibrin in the presence or absence of different amounts of Spike protein. Data are from three independent experiments (mean ±s.e.m.). * P<0.05; ** P<0.01; *** P<0.001 (one-way analysis of variance (ANOVA) with Tukey's multiple comparisons test). FIG. 4H is a schematic diagram illustrating pseudotyping of the SARS-COV-2 spike protein using HIV-1 NL4-3 ΔEnv pro-viral DNA vector. The HIV-1 NL4-3 A Env pro-viral DNA vector was co-transfected with the SARS-COV-2 trimeric Spike glycoprotein expression vector into 293T cells. Forty-eight hours after transfection, the supernatant from the transfected cells was harvested, Spike pseudotyped virions (PVs) were pelleted by ultracentrifugation, and the Spike pseudotyped virions (PVs) were collected.

FIGS. 5A-5E illustrate fibrin(ogen)-dependent SARS COV-2 Spike lung pathology. FIG. 5A shows photomicrographs of mouse lung sections obtained 24 hours after injection of BALD or Spike pseudotyped virions (PVs) and stained with anti-fibrin(ogen) antibody. Nuclei are stained with 4′,6-diamidino-2-phenylindole (DAPI; blue). As illustrated more fibrin(ogen) is detected in these lung sections when the Spike pseudotyped virions (PVs) were present Scale bars, 200 μm (top left panel) and 50 μm (top right panel). Data are from n=6 mice per group (mean γs.e.m.). ** /<0.01 (two-tailed Mann-Whitney test). FIG. SB shows confocal photomicrographs of immunofluorescent double-stained mouse lung sections obtained 24 hours after injection of Spike PVs. Immunoreactivity is shown of Spike (green in the original) and fibrinogen (red in the original) and orthogonal views of the y/z and x/z planes show the localization of fibrinogen with the Spike protein. Scale bar, 50 μm. Representative images from three mice are shown. The scatter plot on the lower right shows that fibrinogen immunoreactivity and Spike immunoreactivity were positively correlated in the 24 images analyzed from n=3 mice, R2=0.496, P=0.0001, Pearson correlation. FIG. 5C shows confocal photomicrographs of mouse lung sections 24 hours after Spike pseudotyped virion (PV) injection showing VCAM-1, fibrinogen, and Spike immunoreactivity. Representative images from three mice are shown. FIG. 5D shows photomicrographs of lung sections from mice 24 hours after injection with BALD, Spike, or HIV-ENV pseudotyped virions and from uninjected healthy controls (un). Immunoreactivity of Mac-2 (green in the original) and gp91-phox (red in the original) are shown. Scale bars, 100 μm. Data were from n=6 mice (with BALD, SARS-COV-2 Spike, or HIV-ENV PVs) and n=3 control mice (uninjected controls) (mean ±s.e.m.). ** P<0.01, *** P<0.001, **** P<0.0001 (one-way ANOVA with Tukey's multiple comparisons test). FIG. 5E shows photomicrographs of the lung from control and Fgo mice showing Mac-2 and gp91-phox immunoreactivity after Spike PV injection The graphs below the photomicrographs illustrate the relative amounts of macrophage (Mac-2) and oxidative stress (gp91-phox) detected. Scale bar, 100 μm Data are from n=6 mice per group (mean±s.e.m.). *** P <0.001, **** P<0.0001 (two-way ANOVA with Tukey's multiple comparisons test).

FIGS. 6A-6C illustrate that the fibrin y377-395 cryptic epitope is required for innate immune activation by SARS COV-2 Spike. FIG. 6A shows photomicrographs of brain sections after control or stereotaxic co-injection of fibrinogen with PBS, BALD, or Spike PVs, showing allograft inflammatory factor 1 (Iba-1) immunoreactivity is detected. Scale bar, 50 μm. The percent area of immunoreactivity in brain sections is quantified in the graph to the right for mice treated as indicated along the x-axis. Data are from n=6 mice per group (mean±s.e.m.). * P<0.05, *** P<0.001, **** P<0.0001 (one-way ANOVA with Tukey's multiple comparisons test). p.s., not significant FIG. 6B illustrates the structure of the fibrinogen carboxyl-terminal γ-chain (white) to the left showing the mapped Spike-binding fibrinogen epitope, γ364-395 (cross-hatched). Sequences are shown to the right are SEQ ID NOs:44-63 and were used for the alanine scanning mutagenesis of peptide γ377-395 blotted with His-tagged Spike protein. The signal intensity of the binding of Spike to sequential Ala substituted peptides is shown in the bar graph to the right. Residues with low signal intensity upon Ala substitution (Bold residues) required for binding included the IIPFXRLXI sequence (SEQ ID NO:64) shown at the top. FIG. 6C shows photomicrographs of lung sections from WT and Fggγ390-396A mice 24 hours after injection of BALD or Spike PVs, showing immunoreactivity for Mac-2 and gp91-phox. Scale bars, 50 μm. Data were from n=6 mice per group (mean±s.e.m.). *** P<0.001. **** P<0 0001 (two-way ANOVA with Tukey's multiple comparisons test).

FIGS. 7A-7E illustrate that fibrin-targeting immunotherapy protects from SARS-COV-2 Spike thromboinflammation. FIG. 7A is a heatmap of fibrin autoantibody ELISA results where the levels of fibrin autoantibody in patents are shown by shaded boxes that were detected at several time points. Longitudinal serum collections across at least one time point were obtained from patients with severe (n=19), mild (n=20) and asymptomatic (n=15) COVID-19 patients. Fibrin autoantibody levels were compared to those of 9 healthy controls and 8 controls with non-COVID respiratory illnesses. White boxes indicate unavailable samples for specific time points (e.g., samples were unavailable for patient 1 at day 15-30 and at day 31-90, and for patient 2 at day 31-90; but samples were available for patients 3-6 at days 0-14). FIG. 7B graphically illustrates whole group sample comparison of fibrin autoantibody levels in samples from patients with severe (n=38), mild (n=39), or asymptomatic (n=31) COVID-19, compared to nine healthy controls and eight controls with non-COVID-19 respiratory illness. Values are mean ±s.e.m. * P=0.0207, **** P<0.0001, *P=0.0121, n.s.=nonsignificant (Welch's ANOVA with Dunnett's multiple comparisons test). FIG. 7C graphically illustrates reactive oxygen species (ROS) production in unstimulated bone marrow-derived macrophages (BMDMs) or BMDMs stimulated for 24 hours with Spike or/and fibrin after 5B8 or lgG2b antibody treatment. Data are from three independent experiments (mean # s.e.m.). As illustrated, higher levels of reactive oxygen species (ROS) were produced by BMDMs when exposed to Spike and fibrin, but treatment with the 5B8 anti-fibrin(ogen) antibodies significantly reduced such ROS production when both Spike and fibrin were present. FIG. 7D shows Mac-2 expression in photomicrographs of lung sections from WT mice injected with Spike PVs, and either 5B8 (30 mg/kg) or IgG2b (30 mg/kg). Scale bar, SO um. The graph to the right summarizes the amounts of macrophages detected per field when the SB8 antibodies or lgG2b antibodies were present. Data are from n=6 mice per group (mean±s.e.m.). ** P<0.01 (two-tailed Mann-Whitney test). FIG. 7E shows gp91-phox detection as a marker of oxidative stress in photomicrographs of lung sections from WT mice injected with Spike PVs, and either 5B8 (30 mg/kg) or lgG2b (30 mg/kg). Scale bar, 50 μm The graph to the right summarizes the gp91-phox-expressing cells per field when the SB8 antibodies or lgG2b antibodies were present. Data are from n=6 mice per group (mean±s.e.m.) ** P<0.01 (two-tailed Mann-Whitney test).

FIGS. 8A-8C illustrate the effects of 5B8 antibody or IgG2b (control) antibody treatment on fibrin(ogen) deposits and Spike accumulation in mice administered SARS-COV-2 Spike pseudovirions. FIG. 8A shows confocal micrograph images of immunofluorescence double immunostained mouse lung sections from SARS-COV-2 Spike pseudovirion injected mice at 24 hours after IgG2b (30 mg/kg) (left panel) or 5B8 (30 mg/kg) (right panel) intravenous administration. Immunoreactivity of spike is bright red while immunoreactivity of Fibrin(ogen) is bright green with concentrated deposits indicated by white arrows. Nuclei were stained with 4′,6-diamidino-2-phenylindole (DAPI; blue). Scale bars, 200 μm. Representative images are shown from n=6 mice per group. FIG. 8B graphically illustrates the quantity of SARS-CoV-2 Spike deposition in mouse lung challenged by SARS-COV-2 Spike pseudotyped virions for 24h after 5B8 antibody (30 mg/kg) or IgG2b antibody (30 mg/kg) intravenous treatment. FIG. 8C graphically illustrates the quantity of fibrin(ogen) deposition in mouse lung challenged by SARS-COV-2 Spike pseudotyped virions for 24 hours after 5B8 antibody (30 mg/kg) or lgG2b antibody (control; 30 mg/kg) intravenous treatment. Data were from n=6 mice per group (mean±s.e.m.). ** p<0.01 (two-tailed Mann-Whitney test).

### DETAILED DESCRIPTION

As described herein anti-fibrin antibodies can significantly reduce the adverse effects of Coronavirus infection, including the short-term and long-term effects of Coronavirus infection. As demonstrated herein, the SARS-COV-2 spike protein can bind fibrinogen/fibrin and increases clot formation and deposition of fibrin in one or more of the lungs, brain, kidneys, gut, or heart However, use of anti-fibrin antibodies can significantly reduce such increases in clot formation and fibrin deposition.

CoVID-19 infection can cause acute and long term complications in patients including pneumonia, trouble breathing (low oxygen blood levels), organ failure in several organs, heart problems, acute respiratory distress syndrome, blood clots, acute kidney injury, bacterial infections, infections by other viruses, and combinations thereof. The symptoms of SARS-COV-2 infection can include inflammation and oxidative stress in organs such as the brain, gut, kidneys, vascular system, lungs or a combination thereof; disruption of the blood brain barrier; and, as illustrated herein increased clot formation and deposition of fibrin in one or more of the lungs, brain, kidneys, gut, or heart.

As illustrated herein, at least some of these symptoms can be reduced, eliminated, and/or prevented by administration of anti-fibrin or anti-fibrinogen antibodies.

Most people who become infected with SARS-COV-2 (COVID-19) recover completely within a few weeks. But some people—even those who had mild versions of the disease—continue to experience symptoms after their initial recovery. These people sometimes describe themselves as “long haulers” and the condition has been called post-CoVID-19 syndrome or “long COVID-19.” As used herein, the long-term adverse effects of SARS-COV-2 infection occur after about 1-3, or 2 weeks after an initial SARS-COV-2 infection. In some cases, the SARS-COV-2 may be detected in these “long haulers” but in other cases the long-term symptoms of SARS-COV-2 infection occur even when the SARS-COV-2 virus is no longer detectable.

As demonstrated herein, anti-fibrin antibodies can effectively inhibit these adverse physiological responses and symptoms of SARS-COV-2 infection. In some cases, anti-fibrin antibodies can inhibit the adverse symptoms of SARS-COV-1 infections.

### Thrombosis and SARS-COV-2 Infection

Persistent life-threatening thrombotic events are a hallmark of COVID-19. Aberrant clots form in multiple organs causing significant morbidity and mortality in COVID-19 patients (Tang et al. J Thromb Haemost 18: 844-847 (2020); Al-Aly et al. Nature 594, 259-264 (2021)). The high incidence of clotting complications has been attributed to disease severity, inflammation and subsequent hypercoagulable state (Merad & Martin, Nat Rev Immunol 20, 355-362 (2020)). However, the clinical picture is puzzling because of disproportionate rates of thrombotic events and abnormal clot properties not observed in other inflammatory conditions, such as severe sepsis or different viral respiratory illnesses (Bouck et al. Arterioscler Thromb Vasc Biol 41, 401-414 (2021); Mitrovic et al. Platelets 32, 690-696 (2021); Merkler et al., JAMA Neurol, (2020); Nalbandian et al., Nat Med 27, 601-615 (2021)). Intriguingly, abnormal clotting is not limited to acutely-ill COVID-19 patients. Pulmonary emboli, stroke and sudden death also occur in young COVID-19 patients with asymptomatic infections or mild respiratory symptoms (Fox et al., Lancet Respir Med 8, 681-686 (2020)). Persistent clotting pathology is prevalent in post-acute 10 sequelae of SARS-COV-2 infection (PASC, Long COVID) (Al-Aly et al. Nature 594: 259-264 (2021); Nalbandian et al., Nat Med 27: 601-615 (2021); Tu et al., JAMA Netw Open 4: e217498 (2021)). The central structural component of blood clots, and a key regulator of inflammation in disease, is insoluble fibrin, which is derived from the blood coagulation factor fibrinogen and is deposited in tissues at sites of vascular damage (Doolittle et al. Ann N Y Acad Sci 936, 31-43 (2001); Davalos & Akassoglou, Semin Immunopatbol 34: 43-62 (2012). Hypercoagulability in COVID-19 is associated with inflammation and the formation of fibrin clots resistant to degradation despite adequate anticoagulation (Merad & Martin, Nat Rev Immunol 20: 355-362 (2020); Bouck et al., Arterioscler Thromb Vasc Biol 41: 401-414 (2021); Mitrovic et al., Platelets 32: 690-696 (2021)). Extensive fibrin deposits are detected locally in inflamed lung and brain tissues from COVID-19 patients, sometimes without evidence of direct viral infection at autopsy (Tang et al., J Thromb Haemost 18: 844-847 (2020); Fox et al. Lancet Respir Med 8: 681-686 (2020); Lee et al. N Engl J Med 384: 481-483 (2021); Thakur et al. Brain (2021); Page & Ariens, Thromb

Res 200, 1-8 (2021)). The high prevalence of thrombotic events with these unique hypercoagulability features suggests an as yet unknown mechanism of abnormal blood clot formation in COVID-19. Experiments described herein were designed to determine how blood clots form in COVID-19 and to identify therapies to combat the deleterious effects of abnormal coagulation occurring in acute and convalescent stages of disease.

### Fibrinogen/Fibrin

Fibrinogen (factor I) is a glycoprotein complex that is made in the liver and that circulates in the blood of vertebrates. During tissue and vascular injury, fibrinogen is converted enzymatically by thrombin to fibrin that can then form a fibrin-based blood clot to occlude blood vessels and stop bleeding. Fibrin can also bind and reduce the activity of thrombin (fibrin is sometimes referred to as antithrombin I), which limits clotting. Fibrin also mediates blood platelet and endothelial cell spreading, tissue fibroblast proliferation, capillary tube formation, and angiogenesis. Fibrin therefore can promote revascularization and wound healing. However, because SARS-COV-2 binds to fibrin, excessive fibrin deposition can contribute to the symptoms of SARS-COV-2 infection.

An example of a human fibrinogen sequence is the fibrinogen gamma chain isoform gamma-A precursor sequence (NCBI accession number NP_000500.2), provided as SEQ ID NO:1 below.

| | |
|---|---|
| 1 | MSWSLHPRNL ILYFYALLFL SSTCVAYVAT RDNCCILDER |
| 41 | FGSYCPTTCG IADFLSTYQT KVDKDLQSLE DILHQVENKT |
| 81 | SEVKQLIKAI QLTYNPDESS KPNMIDAATL KSRKMLEEIM |
| 121 | KYEASILTHD SSIRYLQEIY NSNNQKIVNL KEKVAQLEAQ |
| 161 | CQEPCKDTVQ IHDITGKDCQ DIANKGAKQS GLYFIKPLKA |
| 201 | NQQFLVYCEI DGSGNGWTVF QKRLDGSVDF KKNWIQYKEG |
| 241 | FGHLSPTGTT EFWLGNEKIH LISTQSAIPY ALRVELEDWN |
| 281 | GRTSTADYAM FKVGPEADKY RLTYAYFAGG DAGDAFDGFD |
| 321 | FGDDPSDKFF TSHNGMQFST WDNDNDKFEG NCAEQDGSGW |
| 361 | WMNKCHAGHL NGVYYQGGTY SKASTPNGYD NGIIWATWKT |
| 401 | RWYSMKKTTM KIIPFNRLTI GEGQQHHLGG AKQAGDV |

As illustrated herein antibodies directed against the synthetic fibrin y epitope, CKKTTMKIIPFNRLTIG (SEQ ID NO:2, highlighted above in the SEQ ID NO:1 sequence), are particularly effective at decreasing binding of the SARS-COV-2 spike protein to fibrin and to fibrinogen. Antibodies directed to the SEQ ID NO:2 epitope can also effectively decrease inflammation in a mouse model of Covid-19 induced coagulopathy.

A sequence for a mouse fibrinogen (NCBI accession number NP_001304034.1) is shown below as SEQ ID NO:3.

| | |
|---|---|
| 1 | MSWSLQPPSF LLCCLLLLFS PTGLAYVATR DNCCILDERF |
| 41 | GSFCPTTCGI ADFLSSYQTD VDNDLRTLED ILFRAENRTT |
| 81 | EAKELIKAIQ VYYNPDQPPK PGMIDSATQK SKKMVEEIVK |
| 121 | YEALLLTHET SIRYLQEIYN SNNQKITNLK QKVAQLEAQC |
| 161 | QEPCKDSVQI HDTTGKDCQE IANKGAKESG LYFIRPLKAK |
| 201 | QQFLVYCEID GSGNGWTVLQ KRIDGSLDFK KNWIQYKEGF |
| 241 | GHLSPTGTTE FWLGNEKIHL ISMQSTIPYA LRIQLKDWNG |
| 281 | RTSTADYAMF RVGPESDKYR LTYAYFIGGD AGDAFDGYDF |
| 321 | GDDPSDKFFT SHNGMQFSTW DNDNDKFEGN CAEQDGSGWW |
| 361 | MNKCHAGHLN GVYHQGGTYS KSSTTNGFDD GIIWATWKSR |
| 401 | WYSMKETTMK IIPFNRLSIG EGQQHHMGGS KQVSVDHEVE |
| 441 | IEY |

Note that this mouse fibrinogen has as a slightly different sequence in the region of the human fibrin epitope with SEQ ID NO:2. Other mouse fibrinogen sequences also have sequences that differ from the human fibrinogen sequence in the region of the

SEQ ID NO:2 epitope. The fact that antibodies directed against the human SEQ ID NO:2 epitope indicates that some variation in fibrinogen sequences does not adversely affect the efficacy for decreasing inflammation by anti-fibrinogen antibodies directed against the SEQ ID NO:2 epitope.

Additional epitopes that can be targeted by anti-fibrinogen/fibrin antibodies

Bβ₁₁₉₋₁₂₉ (YLLKDLWQKRQ, SEQ ID NO: 41),
γ₁₆₃₋₁₈₁ (QSGLYFIKPLKANQQFLVY; SEQ ID NO: 42),
γ₃₆₄₋₃₉₅ (DNGIIWATWKTRWYSMKKTTMKIIPFNRLTIG;
SEQ ID NO: 43),

or IIPFXRLXI (SEQ ID NO:64) peptide. The antibodies can bind any of these epitopes.

Isoforms and variants of fibrinogen/fibrin proteins can also be targeted by the antibodies described herein. Such isoforms and variants of fibrinogen/fibrin proteins can have sequences that have between 55-100% sequence identity to any of the fibrinogen/fibrin (reference) sequences described herein.

For example, a human fibrinogen sequence with NCBI accession number AAB59530.1 has the following sequence (SEQ ID NO:68), highlighting the (QSGLYFIKPLKANQQFLVY; SEQ ID NO:42), and γ364-395 (DNGIIWATWKTRWYSMKKTTMKIIPFNRLTIG; SEQ ID NO:43) sequences.

| | |
|---|---|
| 1 | MSWSLHPRNL ILYFYALLFL SSTCVAYVAT RDNCCILDER |
| 41 | FGSYCPTTCG IADFLSTYQT KVDKDLQSLE DILHQVENKT |
| 81 | SEVKQLIKAI QLTYNPDESS KPNMIDAATL KSRIMLEEIM |
| 121 | KYEASILTHD SSIRYLQEIY NSNNQKIVNL KEKVAQLEAQ |
| 161 | CQEPCKDTVQ IHDITGKDCQ DIANKGAKQS GLYFIKPLKA |
| 201 | NQQFLVYCEI DGSGNGWTVF QKRLDGSVDF KKNWIQYKEG |
| 241 | FGHLSPTGTT EFWLGNEKIH LISTQSAIPY ALRVELEDWN |
| 281 | GRTSTADYAM FKVGPEADKY RLTYAYFAGG DAGDAFDGFD |
| 321 | FGDDPSDKFF TSHNGMQFST WDNDNDKFEG NCAEQDGSGW |
| 361 | WMNKCHAGHL NGVYYQGGTY SKASTPNGYD NGIIWATWKT |
| 401 | RWYSMKKTTM KIIPFNRLTI GEGQQHHLGG AKQVRPEHPA |
| 421 | ETEYDSLYPE DDL |

The SEQ ID NO:68 fibrinogen sequence has one amino acid difference compared to the fibrinogen sequence with SEQ ID NO:1.

Isoforms and variants of fibrinogen/fibrin proteins can have at least 55% sequence identity, preferably 60%, preferably 70%, preferably 80%, preferably at least 90%, preferably at least 95%, preferably at least 96%, preferably at least 97% sequence, preferably at least 98%, preferably at least 99% identity to a reference sequence over a specified comparison window. Optimal alignment may be ascertained or conducted using the homology alignment algorithm of Needleman and Wunsch, J. Mol. Biol. 48:443-53 (1970).

### Antibodies

Anti-fibrin and anti-spike antibodies can be used to reduce inflammation associated with SARS-COV-2 infection and to inhibit binding of SARS-COV-2 to fibrin or fibrinogen.

Antibodies can be raised against various epitopes of the fibrinogen, fibrin, SARS-COV-2 Spike protein, or a portion or epitope thereof. Some antibodies for fibrinogen or SARS-COV-2 Spike protein may also be available commercially. However, the antibodies contemplated for treatment pursuant to the methods and compositions described herein are preferably human or humanized antibodies and are highly specific for their fibrinogen/fibrin, or SARS-COV-2 Spike protein targets.

For example, the fibrinogen peptide γ377-395 is the binding site for the CD11b I-domain of complement receptor 3 (CR3) (also known as CD11b/CD18, Mac-1, α_{M}β2) and is required for fibrin-induced activation of microglia and macrophages. A sequence for the CD11b/CD18 (Mac-1) protein is available as accession number P11215-1 from the Uniprot database and shown below as SEQ ID NO:

```
                10 20 30 40
MALRVLLLTA LTLCHGFNLD TENAMTFQEN ARGFGQSVVQ
                50 60 70 80
LQGSRVVVGA PQEIVAANQR GSLYQCDYST GSCEPIRLQV
                90 100 110 120
PVEAVNMSLG LSLAATTSPP QLLACGPTVH QTCSENTYVK
              130 140 150 160
GLCFLFGSNL RQQPQKFPEA LRGCPQEDSD IAFLIDGSGS
              170 180 190 200
IIPHDFRRMK EFVSTVMEQL KKSKTLFSLM QYSEEFRIHF
              210 220 230 240
TFKEFQNNPN PRSLVKPITQ LLGRTHTATG IRKVVRELFN
              250 260 270 280
ITNGARKNAF KILVVITDGE KFGDPLGYED VIPEADREGV
              290 300 310 320
IRYVIGVGDA FRSEKSRQEL NTIASKPPRD HVFQVNNFEA
              330 340 350 360
LKTIQNQLRE KIFAIEGTQT GSSSSFEHEM SQEGFSAAIT
              370 380 390 400
SNGPLLSTVG SYDWAGGVFL YTSKEKSTFI NMTRVDSDMN
              410 420 430 440
DAYLGYAAAI ILRNRVQSLV LGAPRYQHIG LVAMFRQNTG
              450 460 470 480
MWESNANVKG TQIGAYFGAS LCSVDVDSNG STDLVLIGAP
              490 500 510 520
HYYEQTRGGQ VSVCPLPRGR ARWQCDAVLY GEQGQPWGRF
              530 540 550 560
GAALTVLGDV NGDKLTDVAI GAPGEEDNRG AVYLFHGTSG
              570 580 590 600
SGISPSHSQR IAGSKLSPRL QYFGQSLSGG QDLTMDGLVD
              610 620 630 640
LTVGAQGHVL LLRSQPVLRV KAIMEFNPRE VARNVFECND
              650 660 670 680
QVVKGKEAGE VRVCLHVQKS TRDRLREGQI QSVVTYDLAL
              690 700 710 720
DSGRPHSRAV FNETKNSTRR QTQVLGLTQT CETLKLQLPN
              730 740 750 760
CIEDPVSPIV LRLNFSLVGT PLSAFGNLRP VLAEDAQRLF
              770 780 790 800
TALFPFEKNC GNDNICQDDL SITFSFMSLD CLVVGGPREF
              810 820 830 840
NVTVTVRNDG EDSYRTQVTF FFPLDLSYRK VSTLQNQRSQ
              850 860 870 880
RSWRLACESA SSTEVSGALK STSCSINHPI FPENSEVTFN
              890 900 910 920
ITFDVDSKAS LGNKLLLKAN VTSENNMPRT NKTEFQLELP
              930 940 950 960
VKYAVYMVVT SHGVSTKYLN FTASENTSRV MQHQYQVSNL
              970 980 990 1000
GQRSLPISLV FLVPVRLNQT VIWDRPQVTF SENLSSTCHT
            1010 1020 1030 1040
KERLPSHSDF LAELRKAPVV NCSIAVCQRI QCDIPFFGIQ
            1050 1060 1070 1080
EEFNATLKGN LSFDWYIKTS HNHLLIVSTA EILFNDSVFT
            1090 1100 1110 1120
LLPGQGAFVR SQTETKVEPF EVPNPLPLIV GSSVGGLLLL
            1130 1140 1150
ALITAALYKL GFFKRQYKDM MSEGGPPGAE PQ
```

Desirable anti-fibrin/anti-fibrinogen antibodies can block the binding of Mac-1 (CD11b/CD18) to fibrin or fibrinogen. Such antibodies can, for example, block SARS-COV-2-related inflammation by disrupting the fibrin/Mac-1 interaction. The data disclosed herein demonstrates that such anti-fibrin antibodies do in fact reduce inflammation in SARS-COV-2-infected animals.

The SARS-COV-2 spike protein can bind to fibrin as shown herein. As also illustrated herein, the anti-fibrin/anti-fibrinogen antibodies can inhibit binding of the SARS-COV-2 spike protein to fibrin. The spike protein is involved in viral-cell receptor recognition and in fusion of the virus to cell membranes. Binding of SARS-CoV-2 via its spike protein to fibrin may induce inflammation as illustrated herein. However, when anti-fibrin antibodies or similar blocking agents are present such inflammation can be reduced and viral-cellular entry may also be inhibited. One example of a SARS-COV-2 spike protein amino acid sequence is shown below as SEQ ID NO:30.

| | |
|---|---|
| 1 | MFVFLVLLPL VSSQCVNLTT RTQLPPAYIN SFTRGVYYPD |
| 41 | KVFRSSVLHS TQDLFLPFFS NVTWFHAIHV SGINGTKRFD |
| 81 | NPVLPFNDGV YFASTEKSNI IRGWIFGTTL DSKTQSLLIV |
| 121 | NNATNVVIKV CEFQFCNDPF LGVYYHKNNK SWMESEFRVY |
| 161 | SSANNCTFEY VSQPFLMDLE GKQGNFKNLR EFVFKNIDGY |
| 201 | FKIYSKHTPI NLVRDLPQGF SALEPLVDLP IGINITRFQT |
| 241 | LLALHRSYLT PGDSSSGWTA GAAAYYVGYL QPRTFLLKYN |
| 281 | ENGTITDAVD CALDPLSETK CTLKSFTVEK GIYQTSNFRV |
| 321 | QPTESIVRFP NITNLCPFGE VFNATRFASV YAWNRKRISN |
| 361 | CVADYSVLYN SASFSTFKCY GVSPTKLNDL CFTNVYADSF |
| 401 | VIRGDEVRQI APGQTGKIAD YNYKLPDDFT GCVIAWNSNN |
| 441 | LDSKVGGNYN YLYRLFRKSN LKPFERDIST EIYQAGSTPC |
| 481 | NGVEGFNCYF PLQSYGFQPT NGVGYQPYRV VVLSFELLHA |
| 521 | PATVCGPKKS TNLVKNKCVN FNFNGLTGTG VLTESNKKFL |
| 561 | PFQQFGRDIA DTTDAVRDPQ TLEILDITPC SFGGVSVITP |
| 601 | GTNTSNQVAV LYQDVNCTEV PVAIHADQLT PTWRVYSTGS |
| 641 | NVFQTRAGCL IGAEHVNNSY ECDIPIGAGI CASYQTQTNS |
| 681 | PRRARSVASQ SIIAYTMSLG AENSVAYSNN SIAIPTNFTI |
| 721 | SVTTEILPVS MTKTSVDCTM YICGDSTECS NLLLQYGSFC |
| 761 | TQLNRALTGI AVEQDKNTQE VFAQVKQIYK TPPIKDFGGF |
| 801 | NFSQILPDPS KPSKRSFIED LLFNKVTLAD AGFIKQYGDC |
| 841 | LGDIAARDLI CAQKFNGLTV LPPLLTDEMI AQYTSALLAG |
| 881 | TITSGWTFGA GAALQIPFAM QMAYRFNGIG VTQNVLYENQ |
| 921 | KLIANQFNSA IGKIQDSLSS TASALGKLQD VVNQNAQALN |
| 961 | TLVKQLSSNF GAISSVLNDI LSRLDKVEAE VQIDRLITGR |
| 1001 | LQSLQTYVTQ QLIRAAEIRA SANLAATKMS ECVLGQSKRV |
| 1041 | DFCGKGYHLM SFPQSAPHGV VFLHVTYVPA QEKNFTTAPA |
| 1081 | ICHDGKAHFP REGVFVSNGT HWFVTQRNFY EPQIITTDNT |
| 1121 | FVSGNCDVVI GIVNNTVYDP LQPELDSFKE ELDKYFKNHT |
| 1161 | SPDVDLGDIS GINASVVNIQ KEIDRLNEVA KNLNESLIDL |
| 1201 | QELGKYEQYI KWPWYIWLGF IAGLIAIVMV TIMLCCMTSC |
| 1241 | CSCLKGCCSC GSCCKFDEDD SEPVLKGVKL HYT |

The SARS-COV-2 Spike protein is responsible for facilitating entry of the virus into cells. It is composed of a short intracellular tail, a transmembrane anchor, and a large ectodomain that consists of a receptor binding S1 subunit and a membrane-fusing S2 subunit. The spike receptor binding S1 domain can reside at amino acid positions 330-583 of the SEQ ID NO:30 spike protein (shown below as SEQ ID NO:31).

| | |
|---|---|
| 330 | P NITNLCPFGE VFNATRFASV YAWNRKRISN |
| 361 | CVADYSVLYN SASFSTFKCY GVSPTKLNDL CFTNVYADSF |
| 401 | VIRGDEVRQI APGQTGKIAD YNYKLPDDFT GCVIAWNSNN |
| 441 | LDSKVGGNYN YLYRLFRKSN LKPFERDIST EIYQAGSTPC |
| 481 | NGVEGFNCYF PLQSYGFQPT NGVGYQPYRV VVLSFELLHA |
| 521 | PATVCGPKKS TNLVKNKCVN FNFNGLTGTG VLTESNKKFL |
| 561 | PFQQFGRDIA DTTDAVRDPQ TLE |

The entry receptor utilized by SARS-COV-2 is the angiotensin-converting enzyme 2 (ACE-2). The SARS-COV-2 spike protein membrane-fusing S2 domain may be at positions 662-1270 of the SEQ ID NO:30 spike protein (shown below as SEQ ID NO:32).

| | |
|---|---|
| 662 | CDIPIGAGI CASYQTQTNS |
| 681 | PRRARSVASQ SIIAYTMSLG AENSVAYSNN SIAIPINFTI |
| 721 | SVTTEILPVS MTKTSVDCTM YICGDSTECS NLLLQYGSFC |
| 761 | TQLNRALTGI AVEQDKNTQE VFAQVKQIYK TPPIKDFGGF |
| 801 | NFSQILPDPS KPSKRSFIED LLFNKVTLAD AGFIKQYGDC |
| 841 | LGDIAARDLI CAQKFNGLTV LPPLLTDEMI AQYTSALLAG |
| 881 | TITSGWTFGA GAALQIPFAM QMAYRFNGIG VTQNVLYENQ |
| 921 | KLIANQFNSA IGKIQDSLSS TASALGKLQD VVNQNAQALN |
| 961 | TLVKQLSSNF GAISSVLNDI LSRLDKVEAE VQIDRLITGR |
| 1001 | LQSLQTYVTQ QLIRAAEIRA SANLAATKMS ECVLGQSKRV |
| 1041 | DFCGKGYHLM SFPQSAPHGV VFLHVTYVPA QEKNFTTAPA |
| 1081 | ICHDGKAHFP REGVFVSNGT HWFVTQRNFY EPQIITTDNT |
| 1121 | FVSGNCDVVI GIVNNTVYDP LQPELDSFKE ELDKYFKNHT |
| 1161 | SPDVDLGDIS GINASVVNIQ KEIDRLNEVA KNLNESLIDL |
| 1201 | QELGKYEQYI KWPWYIWLGF IAGLIAIVMV TIMLCCMTSC |
| 1241 | CSCLKGCCSC GSCCKFDEDD SEPVLKGVKL H |

A related Spike protein is present in the SARS-COV-1 virus. Such a SARS-CoV-1 Spike protein may also bind fibrinogen or fibrin, causing symptoms similar to SARS-COV-2 symptoms, including fever, cough, and shortness of breath. A sequence for the SARS-COV-1 Spike protein is shown below as SEQ ID NO:33 (NCBI accession no. P59594.1).

| | |
|---|---|
| 1 | MFIFLLFLTL TSGSDLDRCT TFDDVQAPNY TQHTSSMRGV |
| 41 | YYPDEIFRSD TLYLTQDLFL PFYSNVTGFH TINHTFGNPV |
| 81 | IPFKDGIYFA ATEKSNVVRG WVFGSTMNNK SQSVIIINNS |
| 121 | TNVVIRACNF ELCDNPFFAV SKPMGTQTHT MIFDNAFNCT |
| 161 | FEYISDAFSL DVSEKSGNFK HLREFVFKNK DGFLYVYKGY |
| 201 | QPIDVVRDLP SGFNTLKPIF KLPLGINITN FRAILTAFSP |
| 241 | AQDIWGTSAA AYFVGYLKPT TFMLKYDENG TITDAVDCSQ |
| 281 | NPLAELKCSV KSFEIDKGIY QTSNFRVVPS GDVVRFPNIT |
| 321 | NLCPFGEVFN ATKFPSVYAW ERKKISNCVA DYSVLYNSTF |
| 361 | FSTFKCYGVS ATKLNDICFS NVYADSFVVK GDDVRQIAPG |
| 401 | QTGVIADYNY KLPDDFMGCV LAWNTRNIDA TSTGNYNYKY |
| 441 | RYLRHGKLRP FERDISNVPF SPDGKPCTPP ALNCYWPLND |
| 481 | YGFYTTTGIG YQPYRVVVLS FELLNAPATV CGPKLSTDLI |
| 521 | KNQCVNFNFN GLTGTGVLTP SSKRFQPFQQ FGRDVSDFTD |
| 561 | SVRDPKTSEI LDISPCSFGG VSVITPGTNA SSEVAVLYQD |
| 601 | VNCTDVSTAI HADQLTPAWR IYSTGNNVFQ TQAGCLIGAE |
| 641 | HVDTSYECDI PIGAGICASY HTVSLLRSTS QKSIVAYTMS |
| 681 | LGADSSIAYS NNTIAIPTNF SISITTEVMP VSMAKTSVDC |
| 721 | NMYICGDSTE CANLLLQYGS FCTQLNRALS GIAAEQDRNT |
| 761 | REVFAQVKQM YKTPTLKYFG GFNFSQILPD PLKPTKRSFI |
| 801 | EDLLFNKVTL ADAGFMKQYG ECLGDINARD LICAQKFNGL |
| 841 | TVLPPLLTDD MIAAYTAALV SGTATAGWTF GAGAALQIPF |
| 881 | AMQMAYRFNG IGVTQNVLYE NQKQIANQFN KAISQIQESL |
| 921 | TTTSTALGKL QDVVNQNAQA LNTLVKQLSS NFGAISSVLN |
| 961 | DILSRLDKVE AEVQIDRLIT GRLQSLQTYV TQQLIRAAEI |
| 1001 | RASANLAATK MSECVLGQSK RVDFCGKGYH LMSFPQAAPH |
| 1041 | GVVFLHVTYV PSQERNFTTA PAICHEGKAY FPREGVFVFN |
| 1081 | GTSWFITQRN FFSPQIITTD NTFVSGNCDV VIGIINNTVY |
| 1121 | DPLQPELDSF KEELDKYFKN HTSPDVDLGD ISGINASVVN |
| 1161 | IQKEIDRLNE VAKNLNESLI DLQELGKYEQ YIKWPWYVWL |
| 1201 | GFIAGLIAIV MVTILLCCMT SCCSCLKGAC SCGSCCKFDE |
| 1241 | DDSEPVLKGV KLHYT |

A sequence for a portion of the SARS-COV-1 Spike protein is shown below as SEQ ID NO:34 (NCBI accession no. 6WAQ_B).

| | |
|---|---|
| 1 | TNLCPFGEVF NATKFPSVYA WERKKISNCV ADYSVLYNST |
| 41 | FFSTFKCYGV SATKLNDLCF SNVYADSFVV KGDDVRQIAP |
| 81 | GQTGVIADYN YKLPDDFMGC VLAWNTRNID ATSTGNYNYK |
| 121 | YRYLRHGKLR PFERDISNVP FSPDGKPCTP PALNCYWPLN |
| 161 | DYGFYTTTGI GYQPYRVVVL SFEGSLEVLF Q |

Other coronavirus Spike proteins and Spike protein segments have sequences, for example, with NCBI accession numbers BCN86353.1; 6XR8_A; QJF75467.1; QJS39567.1; QJX45031.1; QJR85953.1; QII57278.1; YP_009724390.1; QRN64146.1; QNN86157.1; 7KRQ_A; QJF77846.1; QRN78371.1; QMS52716.1; QIZ16509.1; QMI90807.1; QKU32813.1; QIZ97039.1; QJQ84843.1; QKS90791.1; QIS30425.1; QQP45825.1; QJG65956.1; QMJ01317.1; 6WAQ_B (GI: 1827515989); 6WAQ_D (GI: 1827515987); 6ZDH_C (GI: 1864383468); 6ZDH_B (GI: 1864383467); 6ZDH_A (GI: 1864383466); 7KZB_C (GI: 1972885852); 6ZDG_D (GI: 1881823125); 6ZDG_A (GI: 1881823122); 6ZDG_E (GI: 1881823119); 7LAA B (GI: 2007122781); 6ZFO_A (GI: 1866606289); 6ZFO_E (GI: 1866606286); 6ZCZ_E (GI: 1861314304); 7M3I_R (GI: 2035913025); 7M3I_C (GI: 2035913022); 7LJR_C (GI: 2020309812); 7LJR_B (GI: 2020309811); 7LJR A (GI: 2020309810); 7LAB_C (GI: 2000000810); 7LAB_B (GI: 2000000809); 7LAB_A (GI: 2000000808); 7LCN_K (GI: 1964532181); 7LCN_A (GI: 1964532178); 7LCN_C (GI: 1964532175); 7LAA_C (GI: 2007122784); 7LAA A (GI. 2007122780); 7LD1_C (GI: 1964532188); 7LD1_B (GI: 1964532187); and 7LD1_A (GI: 1964532186)

The anti-Spike antibodies can bind to any of the foregoing Spike proteins, or portions or domains of any of these Spike proteins. In some embodiments, the anti-SARS-COV-2 or anti-SARS-COV-1 Spike antibodies can bind to the region of a Spike protein that binds fibrin or fibrinogen.

The antibodies may be monoclonal antibodies. Such antibodies may also be humanized or fully human monoclonal antibodies. The antibodies can exhibit one or more desirable functional properties, such as high affinity binding to fibrinogen or fibrin, high affinity binding to SARS-COV-2 spike protein, or the ability to inhibit binding of fibrinogen or fibrin to the SARS-COV-2 spike protein.

Methods and compositions described herein can include antibodies that bind fibrinogen or fibrin, or that bind to SARS-COV-2 spike protein. The antibodies can also bind to a combination of antibodies that bind to fibrinogen or fibrin, or a combination where each antibody type can separately bind fibrinogen or fibrin.

The term “antibody” as referred to herein includes whole antibodies and any antigen binding fragment (i.e., “antigen-binding portion”) or single chains thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, CH₁, CH₂ and CH₃. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding portion” of an antibody (or simply “antibody portion”), as used herein, refers to one or more fragments of an antibody that retain the ability to specifically bind to an antigen (e.g. a peptide or domain of fibrinogen or fibrin). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and C_{H1} domains; (ii) a F(ab′)₂ fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

An “isolated antibody,” as used herein, is intended to refer to an antibody that is substantially free of other antibodies having different antigenic specificities (e.g., an isolated antibody that specifically binds fibrinogen or fibrin is substantially free of antibodies that specifically bind antigens other than fibrinogen, fibrin, or the SARs-CoV-2 Spike protein. An isolated antibody that specifically binds fibrinogen or fibrin may, however, have cross-reactivity to other antigens, such as isoforms or related fibrinogen and fibrin proteins from other species. Moreover, an isolated antibody may be substantially free of other cellular material and/or chemicals.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody,” as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody,” as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{L} and V_{H} regions of the recombinant antibodies are sequences that, while derived from and related to human germline V_{L} and V_{H} sequences, may not naturally exist within the human antibody germline repertoire in vivo.

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

The term “human antibody derivatives” refers to any modified form of the human antibody, e.g., a conjugate of the antibody and another agent or antibody.

The term “humanized antibody” is intended to refer to antibodies in which CDR sequences derived from the germline of another mammalian species, such as a mouse, have been grafted onto human framework sequences. Additional framework region modifications may be made within the human framework sequences.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

As used herein, an antibody that “specifically binds to human fibrinogen or fibrin” is intended to refer to an antibody that binds to human fibrinogen or fibrin with a K_{D} of 1×10⁻⁷ M or less, more preferably 5×10⁻⁸ M or less, more preferably 1×10⁻⁸ M or less, more preferably 5×10⁻⁹ M or less, even more preferably between 1×10⁻⁸ M and 1×10⁻¹⁰ M or less.

The term “K_{assoc}” or “Kₐ,” as used herein, is intended to refer to the association rate of a particular antibody-antigen interaction, whereas the term “K_{dis}” or “K_{d},” as used herein, is intended to refer to the dissociation rate of a particular antibody-antigen interaction. The term “K_{D},” as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods well established in the art. A preferred method for determining the K_{D} of an antibody is by using surface plasmon resonance, preferably using a biosensor system such as a Biacore™ system.

The antibodies of the invention are characterized by particular functional features or properties of the antibodies. For example, the antibodies bind specifically to human fibrinogen or fibrin. Preferably, an antibody of the invention binds to fibrinogen or fibrin with high affinity, for example with a K_{D} of 1×10⁻⁷M or less. The antibodies can exhibit one or more of the following characteristics.
(a) binds to human fibrinogen or fibrin with a K_{D} of 1×10⁻⁷M or less;
(b) inhibits the binding of fibrinogen or fibrin to the SARS-COV-2 spike protein;
(c) inhibits the binding of SARS-COV-2 spike protein to fibrinogen or fibrin;
(d) inhibits SARS-COV-2-related inflammation; or
(e) a combination thereof.

For example, the antibodies described herein can prevent greater than 30% binding, or greater than 40% binding, or greater than 50% binding, or greater than 60% binding, or greater than 70% binding, or greater than 80% binding, or greater than 90% binding, or greater than 80% binding of SARS-COV-2 or Mac-1 to fibrinogen.

Assays to evaluate the binding ability of the antibodies to fibrinogen or fibrin can be used, including for example, ELISAs, Western blots and RIAs. The binding kinetics (e.g., binding affinity) of the antibodies also can be assessed by standard assays known in the art, such as by Biacore™. analysis.

When each of the subject antibodies can bind to fibrinogen or fibrin or spike, the V_{L} and V_{H} sequences can be “mixed and matched” to create other binding molecules that bind to fibrinogen or fibrin or spike. The binding properties of such “mixed and matched” antibodies can be tested using the binding assays described above and assessed in assays described in the examples. When V_{L} and V_{H} chains are mixed and matched, a V_{H} sequence from a particular VH/V_{L} pairing can be replaced with a structurally similar V_{H} sequence. Likewise, preferably a V_{L} sequence from a particular VH/V_{L} pairing is replaced with a structurally similar V_{L} sequence.

Accordingly, in one aspect, the invention provides an isolated monoclonal antibody, or antigen binding portion thereof comprising:
a) a heavy chain variable region comprising an amino acid sequence; and
(b) a light chain variable region comprising an amino acid sequence;
wherein the antibody specifically binds fibrinogen or fibrin, or specifically binds spike.

In some cases, the CDR3 domain, independently from the CDR1 and/or CDR2 domain(s), alone can determine the binding specificity of an antibody for a cognate antigen and that multiple antibodies can predictably be generated having the same binding specificity based on a common CDR3 sequence. See, for example, Klimka et al., British J. of Cancer 83(2):252-260 (2000) (describing the production of a humanized anti-CD30 antibody using only the heavy chain variable domain CDR3 of murine anti-CD30 antibody Ki-4); Beiboer et al., J. Mol. Biol. 296:833-849 (2000) (describing recombinant epithelial glycoprotein-2 (EGP-2) antibodies using only the heavy chain CDR3 sequence of the parental murine MOC-31 anti-EGP-2 antibody); Rader et al., Proc. Natl. Acad. Sci. U.S.A. 95:8910-8915 (1998) (describing a panel of humanized anti-integrin alphabeta3 antibodies using a heavy and light chain variable CDR3 domain. Hence, in some cases a mixed and matched antibody or a humanized antibody contains a CDR3 antigen binding domain that is specific for fibrinogen or fibrin.

Described herein are monoclonal antibodies generated in mice that inhibit fibrinogen-SARS-COV-2 binding. In particular, the invention provides monoclonal antibodies that specifically bind the γ³⁷⁷⁻³⁹⁵ epitope of the fibrin and fibrinogen γC domain, or any of the Bβ119-129 (YLLKDLWQKRQ, SEQ ID NO:41), γ163-181 (QSGLYFIKPLKANQQFLVY; SEQ ID NO:42) and γ364-395 (DNGIIWATWKTRWYSMKKTTMKIIPFNRLTIG; SEQ ID NO:43) sites. Such antibodies block the damaging effects of SARS-COV-2 relating to inflammation. These monoclonal antibodies can inhibit binding of fibrin and fibrinogen to the SARS-COV-2 spike protein.

Various polynucleotide and polypeptide sequences related to the 5B8 antibody are described herein. These sequences include the 5B8 light chain amino acid sequence (SEQ ID NO:5), shown below.

| | |
|---|---|
| 1 | TFDSPYQVRR MRFSAQLLGL LVLWIPGSTA DIVMTQAAFS |
| 41 | NPITLGTSAS MSCRSSKSLL HSSGITYLSW YLQKPGQSPQ |
| 81 | LLIYQMSNLA SGVPDRFSSS GSGTDFTLRI SRVEAEDVGV |
| 121 | YYCAQNLELP LTFGAGTKLE LKRADAAPTV SACTKGEF |

Three 5B8 antibody light chain CDR amino acid sequences (CDR-LI, CDR-L2, and CDR-L3), are shown below as SEQ ID NO:6, 7, and 8, respectively.

The CDR-L1 sequence (SEQ ID NO:6) is RSSKSLLHSSGITYLS.

The CDR-L2 sequence (SEQ ID NO:7) is QMSNLAS.

The CDR-L3 sequence (SEQ ID NO:8) is AQNLELPLT.

Three 5B8 antibody heavy chain amino acid sequence is shown below as (SEQ ID NO:9).

| | |
|---|---|
| 1 | NTAFAGFGRN MRSLFSLQLL STQDLAMGWS CIIVLLVSTA |
| 41 | TGVHSQVQLQ QPGAELVRPG TSVKLSCKAS GYTFTSYWIH |
| 81 | WVKQRPGQGL EWIGLIDPSD SYTNYNQKFR GKATLTVDTS |
| 121 | SSTAYMQLSS LTSEDSAVYY CASSDPTGCW GQGTTLTVSP |
| 161 | ASTTPP |

Three heavy chain CDR amino acid sequences (CDR-H1, CDR-H2, and CDR-H3), are shown below as SEQ ID NO:10, 11, and 12, respectively.

The CDR-H1 sequence (SEQ ID NO:10) is GYTFTSYWIH.

The CDR-H2 sequence (SEQ ID NO:11) is LIDPSDSYTNYNQKFR.

The CDR-H3 sequence (SEQ ID NO:12) is SDPTGC.

The 5B8 antibody light chain nucleotide sequence is shown below as SEQ ID NO:13.

| | |
|---|---|
| 1 | ACTTTTGACT CACCATATCA AGTTCGCAGA ATGAGGTTCT |
| 41 | CTGCTCAGCT TCTGGGGCTG CTTGTGCTCT GGATCCCTGG |
| 81 | ATCCACTGCA GATATTGTGA TGACGCAGGC TGCATTCTCC |
| 121 | AATCCAATCA CTCTTGGAAC ATCAGCTTCC ATGTCCTGCA |
| 161 | GGTCTAGTAA GAGTCTCCTA CATAGTAGTG GCATCACTTA |
| 201 | TTTGTCTTGG TATCTGCAGA AGCCAGGCCA GTCTCCTCAG |
| 241 | CTCCTGATTT ATCAGATGTC CAACCTTGCC TCAGGAGTCC |
| 281 | CAGACAGGTT CAGTAGCAGT GGGTCAGGAA CTGATTTCAC |
| 321 | ACTGAGAATT AGCCGAGTGG AGGCTGAGGA TGTGGGTGTT |
| 361 | TATTACTGTG CTCAAAATCT AGAACTTCCG CTCACGTTCG |
| 401 | GTGCTGGGAC CAAGCTGGAG CTGAAACGGG CTGATGCTGC |
| 441 | ACCAACTGTA TCCGCATGCA CCAAGGGCGA ATTC |

The 5B8 antibody heavy chain nucleotide sequence is shown below as SEQ ID NO:14.

| | | | | |
|---|---|---|---|---|
| 1 | GAACACTGCG | TTTGCTGGCT | TTGGAAGAAA | CATGAGATCA |
| 41 | CTGTTCTCTC | TACAGTTACT | GAGCACACAG | GACCTCGCCA |
| 81 | TGGGATGGAG | CTGTATCATT | GTCCTCTTGG | TATCAACAGC |
| 121 | TACAGGTGTC | CACTCCCAGG | TCCAACTGCA | GCAGCCTGGG |
| 161 | GCTGAGCTGG | TGAGGCCTGG | GACTTCAGTG | AAGTTGTCCT |
| 201 | GCAAGGCTTC | TGGCTACACC | TTCACCAGCT | ACTGGATACA |
| 241 | CTGGGTAAAG | CAGAGGCCTG | GACAAGGCCT | TGAGTGGATC |
| 281 | GGACTGATTG | ATCCITCTGA | TAGTTATACT | AACTACAATC |
| 321 | AAAAGTTCAG | GGGCAAGGCC | ACATTGACTG | TAGACACATC |
| 361 | CTCCAGCACA | GCCTACATGC | AGCTCAGCAG | CCTGACATCT |
| 401 | GAGGACTCTG | CGGTCTATTA | CTGTGCAAGC | TCCGATCCTA |
| 441 | CAGGCTGCTG | GGGCCAAGGC | ACCACTCTCA | CAGTCTCCCC |
| 481 | AGCTAGCACA | ACACCCCCA | | |

Nucleotide sequences of the three 5B8 antibody light chain CDRs (CDR-L1, CDR-L2, and CDR-L3), are shown below as SEQ ID NO:15, 16, and 17, respectively.

The 5B8 antibody light chain CDR-L1 nucleotide sequence is:

(SEQ ID NO: 15)
AGGTCTAGTA AGAGTCTCCT ACATAGTAGT GGCATCACTT
ATTTGTCT

The 5B8 antibody light chain CDR-L2 nucleotide sequence is:

(SEQ ID NO: 16)
CAGATGTCCA ACCTTGCCTC

The 5B8 antibody light chain CDR-L3 nucleotide sequence is:

(SEQ ID NO: 17)
GCTCAAAATC TAGAACTTCC GCTCACG

Nucleotide sequences of the three 5B8 antibody heavy chain CDRs (CDR-H1, SEQ ID NO:14; CDR-H2, SEQ ID NO: 15; and CDR-H3, SEQ ID NO:16), are shown below as SEQ ID NO:18, 19, and 20, respectively.

The 5B8 antibody heavy chain CDR-HI nucleotide sequence is:

(SEQ ID NO: 18)
GGCTACACCT TCACCAGCTA CTGGATACAC

The 5B8 antibody heavy chain CDR-H2 nucleotide sequence is:

(SEQ ID NO: 19)
CTGATTGATC CTTCTGATAG TTATACTAAC TACAATCAAA
AGTTCAGGGG C

The 5B8 antibody heavy chain CDR-H3 nucleotide sequence is:

(SEQ ID NO: 20)
TCCGATCCTA CAGGCTGC

In some cases, the methods and compositions described herein can include the 5B8 antibody. In other cases, the methods and compositions described herein do not include the 5B8 antibody.

The sequences provided herein, including the fibrin, fibrinogen, epitope and antibody sequences, are exemplary. Isoforms and variants of these sequences can also be used in the methods and compositions described herein.

For example, isoforms and variants of the proteins and nucleic acids described herein, including the antibody sequences, can be used in the methods and compositions described herein so long as they are substantially identical to the fibrin and antibody sequences described herein. The terms “substantially identity” indicates that a polypeptide or nucleic acid has a sequence with between 55-100% sequence identity to a reference sequence, for example with at least 55% sequence identity, preferably 60%, preferably 70%, preferably 80%, preferably at least 90%, preferably at least 95%, preferably at least 96%, preferably at least 97% sequence, preferably at least 98%, preferably at least 99% identity to a reference sequence over a specified comparison window. Optimal alignment may be ascertained or conducted using the homology alignment algorithm of Needleman and Wunsch, J. Mol. Biol. 48:443-53 (1970).

An indication that two antibody or two polypeptide sequences are substantially identical is that both antibodies or both polypeptides have the same function, for example blocking fibrin binding of the SARS-COV-2 spike protein or blocking inflammation in the brain, gut, kidneys, vascular system, lungs, or a combination thereof. The antibodies that are substantially identical to a 5B8 antibody sequence may not have exactly the same level of activity as the SB8 antibody. Instead, the substantially identical antibody may exhibit greater or lesser levels of binding affinity to fibrin or to the SARS-COV-2 spike protein. For example, the substantially identical antibody or nucleic acid encoding the antibody may have at least about 40%, or at least about 50%, or at least about 60%, or at least about 70%, or at least about 80%, or at least about 90%, or at least about 95%, or at least about 97%, or at least about 98%, or at least about 100%, or at least about 105%, or at least about 110%, or at least about 120%, or at least about 130%, or at least about 140%, or at least about 150%, or at least about 200% of the binding affinity of the 5B8 antibody described herein when measured by similar assay procedures.

### Screening Methods

Also described berein are screening methods that can be used to identify useful small molecules, polypeptides, anti-spike antibodies, anti-fibrin antibodies. Such useful small molecules, polypeptides, and antibodies can be screened for binding fibrin, binding the SARS-COV-2 spike protein, for inhibiting the binding of spike protein to fibrin, for inhibiting binding of Mac-1 and fibrin, or a combination thereof. The small molecules, polypeptides, and antibodies can also be evaluated as therapeutics for treating the short-term and the long-term symptoms of SARS-COV-2 infection. For example, the small molecules, polypeptides, and antibodies can also be tested to ascertain if they can reduce adverse symptoms of SARS-COV-2 infection such as inflammation, oxidative stress, and/or fibrin deposition in the brain, gut, kidneys, vascular system, lungs, or a combination thereof.

Oxidative stress is an imbalance between free radicals and antioxidants in the body. Free radicals include oxygen-containing molecules with an uneven number of electrons. For example, free radicals can include peroxides.

The methods can involve contacting a fibrin, fibrinogen, or spike protein with a test agent and detecting whether the test agent binds to the fibrin, fibrinogen, or spike protein. The methods can also involve detecting whether the test agent binds to a peptide with SEQ ID NO:2, or any of the Bβ119-129 (YLLKDLWQKRQ, SEQ ID NO:41), γ163-181 (QSGLYFIKPLKANQQFLVY; SEQ ID NO:42), γ364-395 (DNGIIWATWKTRWYSMKKTTMKIIPFNRLTIG; SEQ ID NO:43), or IIPFXRLXI (SEQ ID NO:64) peptidyl sites. The test agents, and therapeutic agents, can also bind combinations of these peptides.

In addition, the methods can involve detecting whether a test agent will compete with the 5B8 antibody for binding to fibrin, fibrinogen, or to compete with the spike protein for binding to fibrin or fibrinogen. The methods can also include detecting whether a test agent can inhibit the binding of Mac-1 with fibrin/fibrinogen. Moreover, the methods can involve detecting whether a test agent will compete with the spike protein for binding to fibrin or fibrinogen. Such methods can also involve quantifying the affinity and/or specificity of binding to fibrin, fibrinogen, or spike protein.

Test agents that do bind to fibrin, fibrinogen, or spike protein can also be administered to an animal (e.g., an experimental animal or a model animal) that is infected with SARS-COV-2 virus and then determining whether the test agent can reduce inflammation and/or oxidative stress associated with the SARS-COV-2 infection within the animal. For example, the methods can include determining whether the test agent can reduce inflammation and/or oxidative stress in the brain, gut, kidneys, vascular system, and/or the lungs of animals infected with SARS-COV-2 virus.

### Expression Systems

Nucleic acid segments encoding one or more anti-fibrin antibodies or one or more anti-spike antibodies can be inserted into or employed with any suitable expression system. Commercially useful and/or therapeutically effective quantities of one or more anti-fibrin antibodies or anti-spike antibodies can also be generated from such expression systems.

Recombinant expression of nucleic acids is usefully accomplished using a vector, such as a plasmid. The vector can include a promoter operably linked to nucleic acid segment encoding one or more anti-fibrin antibodies, or encoding one or anti-spike antibodies, or encoding one or antibody fragments.

The vector can also include other elements required for transcription and translation. As used herein, vector refers to any carrier containing exogenous DNA. Thus, vectors are agents that transport the exogenous nucleic acid into a cell without degradation and include a promoter yielding expression of the nucleic acid in the cells into which it is delivered. Vectors include but are not limited to plasmids, viral nucleic acids, viruses, phage nucleic acids, phages, cosmids, and artificial chromosomes.

A variety of prokaryotic and eukaryotic expression vectors suitable for carrying, encoding and/or expressing anti-fibrin antibodies can be used. A variety of prokaryotic and eukaryotic expression vectors suitable for carrying, encoding and/or expressing anti-fibrin antibodies can be employed. Such expression vectors include, for example, pET, pET3d, pCR2.1, pBAD, pUC, and yeast vectors. The vectors can be used, for example, in a variety of in vivo and in vitro situations.

The expression cassette, expression vector, and sequences in the cassette or vector can be heterologous. As used herein, the term “heterologous” when used in reference to an expression cassette, expression vector, regulatory sequence, promoter, coding region, or nucleic acid refers to an expression cassette, expression vector, regulatory sequence, promoter, coding region, or nucleic acid that has been manipulated in some way. For example, a heterologous promoter can be a promoter that is not naturally linked to a nucleic acid of interest, or that has been introduced into cells by cell transformation procedures. A heterologous nucleic acid or promoter also includes a nucleic acid or promoter that is native to an organism but that has been altered in some way (e.g., placed in a different chromosomal location, mutated, added in multiple copies, linked to a non-native promoter or enhancer sequence, etc.). Heterologous nucleic acids may comprise sequences that comprise cDNA forms. Heterologous coding regions can be distinguished from endogenous coding regions, for example, when the heterologous coding regions are joined to nucleotide sequences comprising regulatory elements such as promoters that are not found naturally associated with the coding region, or when the heterologous coding regions are associated with portions of a chromosome not found in nature (e.g., genes expressed in loci where the protein encoded by the coding region is not normally expressed). Similarly, heterologous promoters can be promoters that at linked to a coding region to which they are not linked in nature.

Viral vectors that can be employed include those relating to lentivirus, adenovirus, adeno-associated virus, herpes virus, vaccinia virus, polio virus, AIDS virus, neuronal trophic virus, Sindbis and other viruses. Also useful are any viral families which share the properties of these viruses which make them suitable for use as vectors. Retroviral vectors that can be employed include those described in by Verma, I.M., Retroviral vectors for gene transfer. In Microbiology-1985, American Society for Microbiology, pp. 229-232, Washington, (1985). For example, such retroviral vectors can include Murine Maloney Leukemia virus, MMLV, and other retroviruses that express desirable properties. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral nucleic acid.

A variety of regulatory elements can be included in the expression cassettes and/or expression vectors, including promoters, enhancers, translational initiation sequences, transcription termination sequences and other elements. A “promoter” is generally a sequence or sequences of DNA that function when in a relatively fixed location in regard to the transcription start site. For example, the promoter can be upstream of the nucleic acid segment encoding one or more anti-fibrin antibodies, or a fragment thereof

A “promoter” contains core elements required for basic interaction of RNA polymerase and transcription factors and can contain upstream elements and response elements. “Enhancer” generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 by in length, and they function in cis. Enhancers function to increase transcription from nearby promoters. Enhancers, like promoters, also often contain response elements that mediate the regulation of transcription. Enhancers often determine the regulation of expression.

Expression vectors used in eukaryotic host cells (yeast, fungi, insect, plant, animal, human or nucleated cells) can also contain sequences for the termination of transcription, which can affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs.

The expression of anti-fibrin antibodies or anti-spike antibodies, or antibody fragments thereof from an expression cassette or expression vector can be controlled by any promoter capable of expression in prokaryotic cells or eukaryotic cells. Examples of prokaryotic promoters that can be used include, but are not limited to, SP6, T7, T5, tac, bla, trp, gal, lac, or maltose promoters. Examples of eukaryotic promoters that can be used include, but are not limited to, constitutive promoters, e.g., viral promoters such as CMV, SV40 and RSV promoters, as well as regulatable promoters, e.g., an inducible or repressible promoter such as the tet promoter, the hsp70 promoter and a synthetic promoter regulated by CRE. Vectors for bacterial expression include pGEX-5X-3, and for eukaryotic expression include pCIneo-CMV.

The expression cassette or vector can include nucleic acid sequence encoding a marker product. This marker product is used to determine if a vector or expression cassette encoding the anti-fibrin antibodies has been delivered to the cell and, once delivered, is being expressed. Marker genes can include the E. coli lacZ gene which encodes B-galactosidase, and green fluorescent protein. In some embodiments the marker can be a selectable marker. When such selectable markers are successfully transferred into a host cell, the transformed host cell can survive if placed under selective pressure. There are two widely used distinct categories of selective regimes. The first category is based on a cell's metabolism and the use of a mutant cell line which lacks the ability to grow independent of a supplemented media. The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)).

Gene transfer can be obtained using direct transfer of genetic material, in but not limited to, plasmids, viral vectors, viral nucleic acids, phage nucleic acids, phages, cosmids, and artificial chromosomes, or via transfer of genetic material in cells or carriers such as cationic liposomes. Such methods are well known in the art and readily adaptable for use in the method described herein. Transfer vectors can be any nucleotide construction used to deliver genes into cells (e.g., a plasmid), or as part of a general strategy to deliver genes, e.g., as part of recombinant retrovirus or adenovirus (Ram et al. Cancer Res. 53:83-88, (1993)). Appropriate means for transfection, including viral vectors, chemical transfectants, or physico-mechanical methods such as electroporation and direct diffusion of DNA, are described by, for example, Wolff, J. A. , et al., Science, 247, 1465-1468, (1990); and Wolff, J. A. Nature, 352, 815-818, (1991).

For example, the nucleic acid molecules, expression cassette and/or vectors encoding anti-fibrin antibodies can be introduced to a cell by any method including, but not limited to, calcium-mediated transformation, electroporation, microinjection, lipofection, particle bombardment and the like. The cells can also be expanded in culture and then administered to a subject, e.g. a mammal such as a human. The amount or number of cells administered can vary but amounts in the range of about 10⁶ to about 10⁹ cells can be used. The cells are generally delivered in a physiological solution such as saline or buffered saline. The cells can also be delivered in a vehicle such as a population of liposomes, exosomes or microvesicles.

In some cases, the transgenic cell can produce exosomes or microvesicles that contain nucleic acid molecules, expression cassettes and/or vectors encoding anti-fibrin antibodies, or a combination thereof. In some cases, the transgenic cell can produce exosomes or microvesicles that contain nucleic acid molecules that can target anti-fibrin antibodies to particular tissues. Microvesicles can mediate the secretion of a wide variety of proteins, lipids, mRNAs, and micro RNAs, interact with neighboring cells, and can thereby transmit signals, proteins, lipids, and nucleic acids from cell to cell (see, e.g., Shen et al., J Biol Chem. 286(16): 14383-14395 (2011); Hu et al., Frontiers in Genetics 3 (April 2012); Pegtel et al., Proc. Nat'l Acad Sci 107(14): 6328-6333 (2010); WO/2013/084000; each of which is incorporated herein by reference in its entirety. Cells producing such microvesicles can be used to express the anti-fibrin antibodies.

Transgenic vectors or cells with a heterologous expression cassette or expression vector can express the encoded antibodies or fragments thereof. Any of these vectors or cells can be administered to a subject. Exosomes produced by transgenic cells can also be used to administer anti-fibrin antibody-encoding nucleic acids, anti-spike antibody-encoding nucleic acids, or antibody fragment-encoding nucleic acids to the subject.

Methods and compositions that include antibodies can involve use of one or more types of anti-fibrin antibodies, one or more types of anti-spike antibodies, one or more antibody fragments thereof, or a combination thereof.

### Compositions

The invention also relates to compositions containing the active agents described herein. Such active agents can antibodies, nucleic acids encoding antibodies (e.g., within an expression cassette or expression vector), polypeptides, small molecules, or a combination thereof. The compositions can be pharmaceutical compositions. In some embodiments, the compositions can include a pharmaceutically acceptable carrier. By “pharmaceutically acceptable” it is meant that a carrier, diluent, excipient, and/or salt is compatible with the other ingredients of the formulation, and not deleterious to the recipient thereof.

The composition can be formulated in any convenient form. In some embodiments, the compositions can include antibody, polypeptide, or small molecule that can bind to a SEQ ID NO:2, Bβ119-129 (YLLKDLWQKRQ, SEQ ID NO:41), γ163-181 (QSGLYFIKPLKANQQFLVY; SEQ ID NO:42), γ364-395 (DNGIIWATWKTRWYSMKKTTMKIIPFNRLTIG; SEQ ID NO:43), IIPFXRLXI (SEQ ID NO:64), or a combination of these peptidyl sites. In other embodiments, the compositions can include at least one nucleic acid or expression cassette encoding an antibody or polypeptide that can bind to a SEQ ID NO:2 epitope, or to any of the β119-129 (YLLKDLWQKRQ, SEQ ID NO:41), δ163-181 (QSGLYFIKPLKANQQFLVY; SEQ ID NO:42), β364-395 (DNGIIWATWKTRWYSMKKTTMKIIPFNRLTIG; SEQ ID NO:43), IIPFXRLXI (SEQ ID NO:64), or a combination of these peptidyl sites.

In some embodiments, the active agents of the invention (e.g., antibodies, nucleic acids encoding one or more antibody type (e.g., within an expression cassette or expression vector), polypeptides, small molecules, or a combination thereof), are administered in a “therapeutically effective amount.” Such a therapeutically effective amount is an amount sufficient to obtain the desired physiological effect, such reduction of at least one symptom of SARS-COV-2 infection. For example, active agents can reduce the short-term and the long-term symptoms of CoVID-19 infection such as inflammation, oxidative stress, fibrin deposition, clot formation, clot retention, blood brain barrier deterioration, fatigue, shortness of breath, cough, joint pain, chest pain, or combinations thereof, by 5%, or 10%, or 15%, or 20%, or 25%, or 30%, or 35%, or 40%, or 45%, or 50%, or 55%, or 60%, or 65%, or %70, or 80%, or 90%, 095%, or 97%, or 99%, or any numerical percentage between 5% and 100%.

To achieve the desired effect(s), the active agents may be administered as single or divided dosages. For example, active agents can be administered in dosages of at least about 0 01 mg/kg to about 500 to 750 mg/kg. of at least about 0.01 mg/kg to about 300 to 500 mg/kg, at least about 0.1 mg/kg to about 100 to 300 mg/kg or at least about 1 mg/kg to about 50 to 100 mg/kg of body weight, although other dosages may provide beneficial results. The amount administered will vary depending on various factors including, but not limited to, the type of antibodies, polypeptides, small molecules, or nucleic acid chosen for administration, the severity of the condition, the weight, the physical condition, the health, and the age of the mammal. Such factors can be readily determined by the clinician employing animal models or other test systems that are available in the art.

Administration of the active agents in accordance with the present invention may be in a single dose, in multiple doses, in a continuous or intermittent manner, depending, for example, upon the recipient's physiological condition, whether the purpose of the administration is therapeutic or prophylactic, and other factors known to skilled practitioners. The administration of the active agents and compositions of the invention may be essentially continuous over a preselected period of time or may be in a series of spaced doses. Both local and systemic administration is contemplated. Local administration can be to the heart, lungs, brain, kidneys, gut, liver, muscles, or a combination thereof.

To prepare the antibodies, polypeptides, small molecules, nucleic acids, expression cassettes, and other agents are synthesized or otherwise obtained, purified as necessary or desired. These antibodies, polypeptides, small molecules, nucleic acids, expression cassettes, and other agents can be suspended in a pharmaceutically acceptable carrier and/or lyophilized or otherwise stabilized. The antibodies, polypeptides, small molecules, nucleic acids, expression cassettes, other agents, and combinations thereof can be adjusted to an appropriate concentration, and optionally combined with other desired agents. The absolute weight of a given antibody, polypeptide, small molecule nucleic acid, expression vector, and/or another agent included in a unit dose can vary widely. For example, about 0.01 to about 2 g, or about 0.1 to about 500 mg, of at least one antibody, nucleic acid, polypeptide, small molecule, expression cassette, and/or other agent, or a plurality of antibodies, nucleic acids, polypeptides, small molecules, expression cassettes, and/or other agents can be administered. Alternatively, the unit dosage can vary from about 0.01 g to about 50 g, from about 0.01 g to about 35 g, from about 0.1 g to about 25 g, from about 0.5 g to about 12 g, from about 0.5 g to about 8 g, from about 0.5 g to about 4 g, or from about 0.5 g to about 2 g.

Daily doses of the agents of the invention can vary as well. Such daily doses can range, for example, from about 0.1 g/day to about 50 g/day, from about 0.1 g/day to about 25 g/day, from about 0.1 g/day to about 12 g/day, from about 0.5 g/day to about 8 g/day, from about 0.5 g/day to about 4 g/day, and from about 0.5 g/day to about 2 g/day.

It will be appreciated that the amount of the agent for use in treatment will vary not only with the particular carrier selected but also with the route of administration, the severity of the condition being treated and the age and condition of the patient. Ultimately the attendant health care provider can determine proper dosage. In addition, a pharmaceutical composition can be formulated as a single unit dosage form.

Thus, one or more suitable unit dosage forms comprising the agent(s) can be administered by a variety of routes including parenteral (including subcutaneous, intravenous, intramuscular and intraperitoneal), oral, rectal, dermal, transdermal, intrathoracic, intrapulmonary and intranasal (respiratory) routes. The agent(s) may also be formulated for sustained release (for example, using microencapsulation, see WO 94/07529, and U.S. Pat. No. 4,962,091). The formulations may, where appropriate, be conveniently presented in discrete unit dosage forms and may be prepared by any of the methods available in the pharmaceutical arts. Such methods may include the step of mixing the agents with liquid carriers, solid matrices, semi-solid carriers, finely divided solid carriers or combinations thereof, and then, if necessary, introducing or shaping the product into the desired delivery system. For example, the agent(s) can be linked to a convenient carrier such as a nanoparticle, albumin, polyalkylene glycol, or be supplied in prodrug form. The agent(s), and combinations thereof, can be combined with a carrier and/or encapsulated in a vesicle such as a liposome.

The compositions of the invention may be prepared in many forms that include aqueous solutions, suspensions, tablets, hard or soft gelatin capsules, and liposomes and other slow-release formulations, such as shaped polymeric gels. Administration of active agents can also involve parenteral or local administration of the in an aqueous solution or sustained release vehicle.

Thus, while the agents can sometimes be administered in an oral dosage form, that oral dosage form can be formulated so as to protect the antibodies, polypeptides, small molecules, nucleic acids, expression cassettes, and combinations thereof from degradation or breakdown before the antibodies, polypeptides, small molecules, nucleic acids encoding such polypeptides/antibodies, and combinations thereof provide therapeutic utility. For example, in some cases the antibodies, polypeptides, small molecules, nucleic acids encoding such antibodies/polypeptides, and/or other agents can be formulated for release into the intestine after passing through the stomach. Such formulations are described, for example, in U.S. Pat. No. 6,306,434 and in the references contained therein.

Liquid pharmaceutical compositions may be in the form of, for example, aqueous or oily suspensions, solutions, emulsions, syrups or elixirs, dry powders for constitution with water or other suitable vehicle before use. Such liquid pharmaceutical compositions may contain conventional additives such as suspending agents, emulsifying agents, non-aqueous vehicles (which may include edible oils), or preservatives. The pharmaceutical compositions may take such forms as suspensions, solutions, or emulsions in oily or aqueous vehicles, and may contain formulatory agents such as suspending, stabilizing and/or dispersing agents. Suitable carriers include saline solution, encapsulating agents (e.g, liposomes), and other materials. The agents can be formulated in dry form (e.g., in freeze-dried form), in the presence or absence of a carrier. If a carrier is desired, the carrier can be included in the pharmaceutical formulation, or can be separately packaged in a separate container, for addition to the agents, after packaging in dry form, in suspension, or in soluble concentrated form in a convenient liquid.

Active agent(s) and/or other agents can be formulated for parenteral administration (e.g., by injection, for example, bolus injection or continuous infusion) and may be presented in unit dosage form in ampoules, prefilled syringes, small volume infusion containers or multi-dose containers with an added preservative.

The compositions can also contain other ingredients such as anti-viral agents, antibacterial agents, antimicrobial agents, immune modulators, other monoclonal antibodies, blood thinners, and/or preservatives.

The present description is further illustrated by the following examples, which should not be construed as limiting in any way. The contents of all cited references (including literature references, issued patents, published patent applications as cited throughout this application) are hereby expressly incorporated by reference.

### EXAMPLE 1: MATERIALS AND METHODS

This Example describes some of the materials and methods used in the development of the invention.

### Subjects and Specimens

The COVID-19 infection cohort utilized remnant serum samples from routine clinical laboratory testing at Zuckerberg San Francisco General Hospital (ZSFG). All patients had positive results by SARS-COV-2 real-time polymerase chain reaction (RT-PCR) in nasopharyngeal swabs between March and July 2020. Clinical data were extracted from electronic health records and included demographic information, major co-morbidities, patient-reported symptom onset date, symptoms, and indicators of disease severity. COVID-19 was classified as severe in nineteen patients (admitted to intensive care unit), mild in twenty patients (admitted to hospital or managed as outpatients), and as asymptomatic in fifteen patients (not hospitalized, no symptoms). The criteria for ICU admission at the hospital remained the same throughout the course of the study. Non-COVID respiratory illness control (n=8) remnant samples were from febrile patients with upper respiratory symptoms who were SARS-COV-2 RT-PCR negative. The protocol for ZSFG remnant specimen collection from patients with suspected COVID-19 infection (IRB #20-30387) was approved by the University of California, San Francisco (UCSF) Institutional Review Board. The committee judged that written consent was not required for use of remnant specimens. Whole blood from healthy control were obtained from subjects enrolled at UCSF for a study that predates the COVID-19 outbreak. Human subjects research was approved by the UCSF Institutional Review Board (IRB# 10-00650). All subjects provided written informed consent before participation in this study. Ten ml whole blood was collected from each healthy control subject into Vacutainer tubes without anticoagulant, and blood was allowed to coagulate for 30 minutes. Tubes were centrifuged at 1300× g for 30 min. Serum layer was harvested, aliquoted, and preserved at −80° C.

### Animals

CS7BL/6 mice were purchased from the Jackson Laboratory. Fgα^{-/-} (Suh et al., Genes Dev 9: 2020-2033 (1995)) and Fggγ390-396A mice (2) were obtained from Dr. Jay Degen (University of Cincinnati, OH, USA) Mice were housed under a 12:12 light/dark cycle, 55%±5% relative humidity, and a temperature of 20±2° C. with access to standard laboratory chow and water ad libitum. They were boused in social groups of a maximum of 5 mice in standard mouse housing cages and bedding.

All single-housed mice were provided with cage enrichment (a cardboard or hard-plastic house-like hiding place and tissue paper). For husbandry, one male and one female were housed together with a maximum of one litter was permitted. Mice were weaned at postnatal day 21. Male mice were used for all experiments. All animal procedures were performed under the guidelines set by the Institutional Animal Care and Use Committee at the University of California, San Francisco

### SARS-COV-2 Recombinant Trimeric Spike Protein Production in a Mammalian Expression System

The plasmid vector pCAGGS containing the SARS Coronavirus 2, Wuhan-Hu-1 ectodomain Spike glycoprotein gene with a deletion of the polybasic cleavage site (RRAR to A), two stabilizing mutations (K986P and V987P), a C-terminal thrombin cleavage site, T4 fold on trimerization domain, and a hexahistidine tag (6xHis) was obtained from BEI Resources (deposited by the laboratory of Dr. Florian Krammer) (Stadlbauer et al. Curr Protoc Microbiol 57: e100 (2020)). Recombinant Spike protein was produced by Celltheon (Union City, CA). Briefly, CHO cells were transiently transfected with the plasmid and harvested at >70% viability. Spike protein was obtained by centrifugation and sterile filtration, purified by Ni2+-NTA affinity chromatography, and eluted in phosphate-buffered saline (PBS) containing imidazole. Fractions containing eluted recombinant spike protein were then buffer exchanged into 1× PBS and was further purified by size-exclusion chromatography using a Superdex 200 column.

### Fibrin Polymerization Assay

Fibrin polymerization was measured by turbidity analysis as described (Ryu et al. Nat Immunol 19: 1212-1223 (2018)). In brief, pooled healthy donor citrated human plasma (Innovative Research) was diluted to 1:3 in 20 mM HEPES. Recombinant trimeric spike protein was freshly thawed without freezing and thaw cycles. Recombinant trimeric spike protein was buffer exchanged into 20 mM HEPES, pH 7.4, 137 mM NaCl using Amicon concentrators (100 kDa cut-off) prior to plasma incubation. 50 μl of plasma dilution was incubated with 50 μl recombinant trimeric spike protein at 25° C. for 15 min. Clotting was initiated by 0.25 U/ml thrombin (Sigma-Aldrich) and 20 mM CaCl2. Final concentrations were 1:12 plasma, 0.75 μM Spike, 0.25 U/ml thrombin, 20 mM CaCl₂. Turbidity was measured at 340 nm every 15 sec for 30 min in a SpectraMax MS microplate reader (Molecular Devices) with SoftMax Pro 5.2 software (Phoenix Technologies).

### Scanning Electron Microscopy (SEM) of Fibrin Clots

Healthy donor citrated human plasma was diluted 1:3 in 20 mM HEPES buffer, pH 7.4; 15 μl of the diluted plasma was mixed with 15 μl of recombinant trimeric spike protein that was buffer exchanged into 20 mM HEPES and 137 mM NaCl using Amicon concentrators (100 kDa cut-off) prior to addition to plasma. Low concentration of NaCl was used to maintain spike solubility and stability Then, 25 μl of this mixture was pipetted onto 5 mm×5 mm silicon wafers (Ted Pella) and incubated for 15 min at 37° C. in a humidified tissue culture incubator. Next, 25 μl of a solution of CaCla and thrombin in 20 mM HEPES was added in the center of the wafer and allowed to polymerize at 25° ° C. for 2 hour. Final concentrations were plasma 1:12, 0.9 μM Spike, 0.25 U/ml thrombin, 20 mM CaCh Buffer was used instead of spike for control condition. Clots on wafers were placed on ice, washed twice for 10 min each with ice-cold electron microscopy grade 0.1 M cacodylate buffer, pH 7.4, and fixed in cold electron microscopy grade 2% glutaraldehyde (Electron Microscopy Sciences). Samples were rinsed three times for 5 min each in Millipore-filtered, double-distilled water; dehydrated in an ethanol series (20%, 50%, 70%, 90%, 100%, 100% for 2 min each); and critical point dried with carbon dioxide. Samples were sputter coated with a thin layer of gold-palladium and imaged with a Zeiss Merlin field-emission scanning electron microscope at 3.0 keV and a secondary electron detector.

SEM imaging and image acquisition were carried out blinded to test conditions. 4000X images were captured across the sample, then were converted to 8-bit with NIH ImageJ (v. 1.50). After pixel to micron scaling, each image was cropped into two to three fields of view (FOV) (8×8 μm) with NIH DiameterJ as described (Hotaling et al. Biomaterials 61, 327-338 (2015)). Surface plot plug-in ImageJ was used to generate topographical maps of SEM images. Briefly, the best segmentation algorithm was pre-selected based on side by side comparison of images before quantification. The Mixed Segmentation (M1-through M3 options) built in DiameterJ Segment provided the most accurate representation of the fibers to be quantified. The same segmentation method and variant was used across all test conditions and images.

Each segmented image was manually edited with ImageJ to ensure complete representation of segmented fibers. Edited images were batch processed with DiameterJ 1-108 (orientation analysis not selected). Fiber radius and intersection densities were collated from each batch. Data from 8-10 FOVs per sample was generated for group analysis.

### Fibrinogen and Fibrin Coated ELISA PLATES

Fibrinogen and fibrin coated plates were prepared as described (4). Briefly, human plasminogen-free fibrinogen (EMD Millipore) was used after IgG depletion using a Pierce albumin/IgG removal kit (Thermo Fisher Scientific). IgG-depleted human plasminogen-free fibrinogen was further diluted to 25 μg/ml by adding 20 mM HEPES buffer, pH 7.4 for coating fibrinogen plates or 20 mM HEPES buffer pH 7.4 with 1U/ml thrombin (Sigma-Aldrich) and 7 mM CaCl₂ for fibrin coated plates. Coating was performed for 1.5 h at 37° C. using 96-well MaxiSorp plates (Thermo Fisher Scientific) and fibrin-coated plates were dried at 37° C. overnight as described by Ryu et al. (Nat Immunol 19: 1212-1223 (2018)).

### Recombinant SARS-COV-2 Spike Protein Binding on Fibrin or Fibrinogen

Fibrin- or fibrinogen-coated 96-well plates were washed with wash buffer (1×PBS+0.05% Tween-20) and incubated with blocking buffer consisting of wash buffer with 5% bovine serum albumin (BSA) (Omnipure, Fisher) for 1 h at 25° C. Serial dilutions of recombinant spike were made in binding buffer (wash buffer containing 0.5% BSA). Recombinant trimeric spike protein was added to the wells and incubated for 2 h at 37° C. After washing five times with binding buffer, rabbit polyclonal anti-6x His tag antibody (1:1000, abcam, ab 137839) was added to the plates and incubated for 1 h at 25° C. Following washing, goat anti-rabbit IgG H&L (conjugated with horse radish peroxidase, HRP) (1:1000, abcam, ab205718) in wash buffer was added for 1 h at 25° C. After the final wash, the HRP substrate 3,3′,5,5′-tetramethybenzidine (TMB; Sigma-Aldrich) was added into the wells. The reaction was quenched by adding IN hydrochloric acid, and absorption was measured at 450 om. Non-linear regression curves were analyzed using Prism 9 software to calculate Kd values using one site binding model.

### Fibrinogen Peptide Array and Spike Binding Epitope Mapping

A custom PepStar™ Multiwell fibrinogen Peptide array that comprises a purified synthetic peptide library containing 390 15-mer peptides representing overlapping peptide scans (15/11) of the α, β, and γ fibrinogen chains (UniProt IDs: FIBA P02671, FIBB P02675, FIBG P02679) was generated by JPT Peptide Technologies (Berlin, Germany). The arrays were hybridized with Recombinant-His tagged trimeric Spike protein (1 μg/ml in blocking buffer) for 1 hour at 30° C. The His-tag peptide (AGHHHHHH (SEQ ID NO:65) was co-immobilized on the peptide microarray slides as an assay control. Microarrays were incubated for 1 hour at 30° C. with fluorescently labeled anti-6xHis monoclonal antibody (Alexa 647, Invitrogen, MA1-135-A647) diluted to 1 μg/ml in blocking buffer and dried. Before each step, microarrays were washed with washing buffer, 50 mM TBS-buffer including 0.1% Tween20, pH 7.2. The assay buffer was LowCross buffer (Candor Bioscience). The slides were washed, dried, and scanned with a high-resolution laser scanner at 635 nm to obtain fluorescence intensity profiles. The images were quantified to yield a mean pixel value for each peptide All incubations were done in 1 day. To assess nonspecific binding to the peptides and assay performance, a control incubation with secondary antibody only (no sample present) was done in parallel on each slide. The resulting images were analyzed and quantified with spot recognition software (GenePix, Molecular Devices). For each spot, the mean signal intensity was extracted (between 0 and 65535 arbitrary units). To visualize the results, heatmap diagrams representing all peptides immobilized on the microarray and containing all signal values were computed; fluorescence intensities were color-coded from white (no binding), to yellow (medium binding), to red (strong binding). Binding peptides were further mapped onto the 3D crystal structure of fibrinogen (PDB ID: 3GHG) with UCSF Chimera (Pettersen et al. J Comput Chem 25: 1605-1612 (2004)).

### Peptide Alanine Scanning

Alanine scanning experiments were performed with PepStar™ Multiwell microarrays containing 60 peptides representing Alanine substitutions of each residue on peptide YSMKKTTMKIIPFNRLTIG (SEQ ID NO:44) by JPT Peptide Technologies. Human full-length IgG and His-tagged peptides were co-immobilized on the peptide microarray slides as assay controls. His-tagged spike protein was applied at five concentrations (from 10 μg/ml to 0.001 μg/ml) and incubated for 1 hour at 30° C. Two fluorescently labeled secondary antibodies specific to the His tag were added separately and left to react for 1 hour. After washing and drying, the slides were scanned with a high-resolution laser scanner at 635 nm to obtain the fluorescence intensity profiles, and the images were quantified to yield a mean pixel value for each peptide. Control incubations with each secondary antibody only (no sample present) were performed in parallel on the slide to assess nonspecific binding to the peptides and assay performance. The data was analyzed with respect to the original peptide. A higher signal after alanine substitution indicated that a residue was not involved in binding to spike protein while a lower signal indicated that a residue was important for binding to spike protein.

### Plasmin Digestion of Fibrin Clots

Before clotting, 3 μM fibrinogen as incubated with 9 μM recombinant trimeric spike protein at 37° C. for 1 hour in 20 mM HEPES, pH 7.4, 137 mM NaCl, 5 mM CaCl₂. Thrombin was added to the mixture at a final concentration of 1.5 U/ml. Fibrin clots were allowed to form in Eppendorf tubes over a 2-h incubation at 37° C. Then, 5 μl of 100 ng/ml plasmin (Millipore) was added to each tube on top of the clot. All samples were incubated at 37° C. for 0, 1, 2, 4, and 6 hours; digestion was quenched by adding sodium dodecyl sulfate-polyacrylamide gel electrophoresis

(SDS-PAGE loading buffer with reducing regent. Samples were heated at 85° C. for 20 min, and aliquots (equivalent to 100 ng fibrinogen) were separated by SDS-PAGE on 4-12% Bis-Tris gels, transferred to PVDF membranes, and analyzed for anti-human fibrinogen by western blot. Band intensities of each protein species (i.e., γ-γ dimer, β-chain) were analyzed with Image J and normalized to corresponding bands at the 0 h time point.

### ROS Detection

Bone marrow-derived macrophages (BMDM) culture and ROS detection using 5 pM DHE (Invitrogen) were performed as described (Ryu et al. Nat Immunol 19: 1212-1223 (2018); Mendiola et al. Nat Immunol 21: 513-524 (2020)). Briefly, cells were plated on 96-well black p-clear-bottomed microtiter plates (Greiner Bio-One) pre-coated with 12.5 μg/ml fibrin with or without recombinant trimeric spike protein (0.168, 1.68, and 3.36 M). For fibrin inhibition, 5B8 or IgG2b (each 20 ug/ml) (MPC-11, BioXCell) was added in fibrin with or without 3.36 μM recombinant trimeric spike protein-coated wells 2 hours before plating of cells. Cells were incubated on fibrin for 24 hours and DHE fluorescence was detected at 518 nm/605 nm with a SpectraMax M5 microplate reader. Since macrophage activation can be influenced by cell culture conditions, heat-inactivated fetal bovine serum and macrophage colony-stimulating factor were batch tested as described (Mendiola et al. Nat Immunol 21: 513-524 (2020)).

### Production of Recombinant Monomeric Spike and Monomeric Spike Deletion Mutants in E. coli

A plasmid expressing full-length Spike (amino acids (aa) 1-1273) of SARS-CoV-2, Wuhan-Hu-1 (GenBank: MN908947; SEQ ID NO:30) with a C-terminal 6xHis was generated by amplifying the Spike coding sequence and inserting it into pET-21a(+) (Novagen) at BamHI/XhoI sites.

Plasmids expressing six Spike mutants-S1 (aa 1-685), S1ΔCT (aa 1-541), S1NT (aa 1-318), receptor binding domain (aa 319-541), S1ΔNT (aa 319-685), and S2 (aa 686-1273)-were generated with a PCR-based method and properly mutated primers.

The S1 (aa 1-685) mutant has the following sequence (SEQ ID NO:35).

| | | | | |
|---|---|---|---|---|
| 1 | MFVFLVLLPL | VSSQCVNLTT | RTQLPPAYIN | SFTRGVYYPD |
| 41 | KVERSSVLHS | TQDLFLPFFS | NVTWFHAIHV | SGINGTKRED |
| 81 | NPVLPENDGV | YFASTEKSNI | IRGWIFGTTL | DSKTQSLLIV |
| 121 | NNATNVVIKV | CEFQFCNDPF | LGVYYHKNNK | SWMESEFRVY |
| 161 | SSANNCTFEY | VSQPFLMDLE | GKQGNEKNLR | EFVEKNIDGY |
| 201 | FKIYSKHTPI | NLVRDLPQGF | SALEPLVDLP | IGINITRFQT |
| 241 | LLALHRSYLT | PGDSSSGWTA | GAAAYYVGYL | QPRTELLKYN |
| 281 | ENGTITDAVD | CALDPLSETK | CTLKSFTVEK | GIYQTSNERV |
| 321 | QPTESIVRFP | NITNLCPFGE | VENATRFASV | YAWNRKRISN |
| 361 | CVADYSVLYN | SASFSTEKCY | GVSPTKLNDL | CFTNVYADSE |
| 401 | VIRGDEVRQI | APGQTGKIAD | YNYKLPDDFT | GCVIAWNSNN |
| 441 | LDSKVGGNYN | YLYRLERKSN | LKPFERDIST | EIYQAGSTPC |
| 481 | NGVEGENCYF | PLQSYGFQPT | NGVGYQPYRV | VVLSFELLHA |
| 521 | PATVCGPKKS | TNLVKNKCVN | FNENGLTGTG | VLTESNKKEL |
| 561 | PFQQFGRDIA | DTTDAVRDPQ | TLEILDITPC | SFGGVSVITP |
| 601 | GTNTSNQVAV | LYQDVNCTEV | PVAIHADQLT | PTWRVYSTGS |
| 641 | NVFQTRAGCL | IGAEHVNNSY | ECDIPIGAGI | CASYQTQTNS |
| 681 | PRRAR | | | |

The S1ΔCT (aa 1-541) mutant has the following sequence (SEQ ID NO:36).

| | | | | |
|---|---|---|---|---|
| 1 | MFVFLVLLPL | VSSQCVNLIT | RTQLPPAYIN | SFTRGVYYPD |
| 41 | KVFRSSVLHS | TQDLFLPFFS | NVTWFHAIHV | SGTNGTKRED |
| 81 | NPVLPENDGV | YFASTEKSNI | IRGWIFGTTL | DSKTQSLLIV |
| 121 | NNATNVVIKV | CEFQFCNDPF | LGVYYHKNNK | SWMESEFRVY |
| 161 | SSANNCTFEY | VSQPFLMDLE | GKQGNEKNLR | EFVEKNIDGY |
| 201 | FKIYSKHTPI | NLVRDLPQGF | SALEPLVDLP | IGINITRFQT |
| 241 | LLALHRSYLT | PGDSSSGWTA | GAAAYYVGYL | QPRTELLKYN |
| 281 | ENGTITDAVD | CALDPLSETK | CTLKSFTVEK | GIYQTSNERV |
| 321 | QPTESIVRFP | NITNLCPFGE | VENATRFASV | YAWNRKRISN |
| 361 | CVADYSVLYN | SASFSTEKCY | GVSPTKINDL | CFTNVYADSE |
| 401 | VIRGDEVRQI | APGQTGKIAD | YNYKLPDDFT | GCVIAWNSNN |
| 441 | LDSKVGGNYN | YLYRLFRKSN | LKPFERDIST | EIYQAGSTPC |
| 481 | NGVEGENCYF | PLQSYGFQPT | NGVGYQPYRV | VVLSFELLHA |
| 521 | PATVCGPKKS | TNLVKNKCVN | F | |

The SINT (aa 1-318) mutant has the following sequence (SEQ ID NO:37).

| | | | | |
|---|---|---|---|---|
| 1 | MFVFLVLLPL | VSSQCVNLIT | RTQLPPAYTN | SFTRGVYYPD |
| 41 | KVERSSVLHS | TQDLFLPFFS | NVTWFHAIHV | SGINGTKRED |
| 81 | NPVLPENDGV | YFASTEKSNI | IRGWIFGTTL | DSKTQSLLIV |
| 121 | NNAINVVIKV | CEFQFCNDPF | LGVYYHKNNK | SWMESEFRVY |
| 161 | SSANNCTFEY | VSQPFLMDLE | GKQGNEKNLR | EFVEKNIDGY |
| 201 | FKIYSKHTPI | NLVRDLPQGF | SALEPLVDLP | IGINITRFQT |
| 241 | LLALHRSYLT | PGDSSSGWTA | GAAAYYVGYL | QPRTFLLKYN |
| 281 | ENGTITDAVD | CALDPLSETK | CTLKSFTVEK | GIYQTSNE |

The receptor binding domain (aa 319-541) mutant has the following sequence (SEQ ID NO:38).

| | | | | |
|---|---|---|---|---|
| 319 | | | | RV |
| 321 | QPTESIVREP | NITNLCPFGE | VENATRFASV | YAWNRKRISN |
| 361 | CVADYSVLYN | SASFSTFKCY | GVSPTKLNDL | CFTNVYADSE |
| 401 | VIRGDEVRQI | APGQTGKIAD | YNYKLPDDFT | GCVIAWNSNN |
| 441 | LDSKVGGNYN | YLYRLFRKSN | LKPFERDIST | EIYQAGSTPC |
| 481 | NGVEGENCYF | PLQSYGFQPT | NGVGYQPYRV | VVLSFELLHA |
| 521 | PATVCGPKKS | TNLVKNKCVN | F | |

The S1ΔNT (aa 319-685) mutant has the following sequence (SEQ ID NO:39)

| | | | | |
|---|---|---|---|---|
| 319 | | | | RV |
| 321 | QPTESIVREP | NITNLCPFGE | VENATRFASV | YAWNRKRISN |
| 361 | CVADYSVLYN | SASFSTEKCY | GVSPTKINDL | CFTNVYADSE |
| 401 | VIRGDEVRQI | APGQTGKIAD | YNYKLPDDFT | GCVIAWNSNN |
| 441 | LDSKVGGNYN | YLYRLERKSN | LKPFERDIST | EIYQAGSTPC |
| 481 | NGVEGENCYF | PLQSYGFQPT | NGVGYQPYRV | VVLSFELLHA |
| 521 | PATVCGPKKS | TNLVKNKCVN | FNENGLIGTG | VLTESNKKEL |
| 561 | PFQQFGRDIA | DTTDAVRDPQ | TLEILDITPC | SFGGVSVITP |
| 601 | GTNTSNQVAV | LYQDVNCTEV | PVAIHADQLT | PTWRVYSTGS |
| 641 | NVFQTRAGCL | IGAEHVNNSY | ECDIPIGAGI | CASYQTQTNS |
| 681 | PRRAR | | | |

The S2 (aa 686-1273) mutant has the following sequence (SEQ ID NO:40).

| | | | | |
|---|---|---|---|---|
| 681 | SVASQ | SIIAYTMSLG | AENSVAYSNN | SIAIPTNFTI |
| 721 | SVTTEILPVS | MTKTSVDCTM | YICGDSTECS | NLLLQYGSFC |
| 761 | TQLNRALTGI | AVEQDKNTQE | VFAQVKQIYK | TPPIKDEGGE |
| 801 | NFSQILPDPS | KPSKRSFIED | LLENKVTLAD | AGFIKQYGDC |
| 841 | LGDIAARDLI | CAQKENGLTV | LPPLLTDEMI | AQYTSALLAG |
| 881 | TITSGWTFGA | GAALQIPFAM | QMAYRENGIG | VTQNVLYENQ |
| 921 | KLIANQFNSA | IGKIQDSLSS | TASALGKLQD | VVNQNAQALN |
| 961 | TLVKQLSSNF | GAISSVLNDI | LSRLDKVEAE | VQIDRLITGR |
| 1001 | LQSLQTYVTQ | QLIRAAEIRA | SANLAATKMS | ECVLGQSKRV |
| 1041 | DFCGKGYHLM | SFPQSAPHGV | VELHVTYVPA | QEKNFTTAPA |
| 1081 | ICHDGKAHFP | REGVFVSNGT | HWFVTQRNFY | EPQIITTDNT |
| 1121 | FVSGNCDVVI | GIVNNTVYDP | LQPELDSFKE | ELDKYFKNHT |
| 1161 | SPDVDLGDIS | GINASVVNIQ | KEIDRLNEVA | KNLNESLIDL |
| 1201 | QELGKYEQYI | KWPWYIWLGF | IAGLIAIVMV | TIMLCCMTSC |
| 1241 | CSCLKGCCSC | GSCCKEDEDD | SEPVLKGVKL | HYT |

The expression plasmids encoding these mutant spike proteins were transformed into E. coli Rosetta 2(DE3) pLysS competent cells (Novagen) and cultured in 500-ml flasks containing 100 ml of LB + ampicillin and chloramphenicol on a shaking incubator (250 rpm, 37° C. ) to optical density of 0.4 at 600 ng. The cultures were induced with 1 mM IPTG (Thermo Fisher Scientific), incubated for 4 hours, and centrifuged (6000 g, 4° C.) for 15 min. The supernatant was removed, and the pellets were frozen at −80° C. overnight. Spike produced in E.coli was only used for immunoprecipitation assays.

### Immunoprecipitation Assay

For co-immunoprecipitation to test interaction of fibrinogen with Spike protein (His-tagged recombinant trimeric spike protein or monomeric Spike protein produced in E. coli), the Pierce co-immunoprecipitation kit (Thermo Fisher Scientific) protocol was used with an original immunoprecipitation/lysis buffer and modifications. For lysis, the frozen cell pellets were solubilized in 800 μl of immunoprecipitation/lysis buffer (50 mM Tris, pH 8 0, 5% glycerol, 1% NP-40, 100 mM NaCl) supplemented with 100 μg/ml lysozyme (Sigma-Aldrich), 100× EDTA-free Halt protease inhibitor (Thermo Fisher Scientific), and 250 U/ul benzonase nuclease (Sigma-Aldrich). E. coli cells were lysed by two rounds of sonication at 30 Hz for 30 sec each until the sample was no longer viscous. After further mixing for 20 min in a rotator, the lysate was cleared by centrifugation at 10,000 g for 10 min, warmed to 37° C. , mixed with 25 μg of fibrinogen, incubated for 1 hour, applied to beads incubated with 10 μg of anti-fibrinogen sheep antibody (SAFGAP, Enzyme Research Laboratories), and incubated for 1 h at 37° C. All washes were done essentially as described in the kit The bound proteins were eluted in 60 μl of EB solution provided in the kit and neutralized with 1/10 volume of 1 M Tris, pH 9.0. Washing buffer and EB solution were warmed to 37° C. in advance The eluted proteins were separated by SDS-PAGE on 4-12% gels, transferred to PVDF membranes (Invitrogen), and incubated with rabbit anti-His antibody (1:1000, Cell Signaling, 2365S) and sheep anti-fibrinogen antibody (1:1000, Enzyme Research Laboratories, SAFG-AP) and then with HRP-conjugated anti-rabbit (111-035-144, Jackson ImmunoResearch; 1:10,000) and anti-sheep (HAF016, R&D Systems; 1:5000) secondary antibodies. Protein bands were detected with Immobilon Forte Western HRP substrate (Sigma-Aldrich) and the ChemiDoc imaging system (Bio-Rad).

### Production of Spike Pseudotyped Virions (PVs)

For production of HIV virions pseudotyped with SARS-COV-2 trimeric Spike glycoprotein (Spike PVs), 293T cells (3.75×106) were plated in a T175 flask and transfected 24 h later with 90 μg of polyethyleneimine (PEI), 30 μg of HIV-1 NL-4-3 Δ Env eGFP (NIH AIDS Reagent Program), or 3.5 μg of pCAGGS SARS-COV-2 trimeric Spike glycoprotein (NR52310, BEI Resources) in a total of 10 ml of Opti-MEM medium (Invitrogen), using PEI transfection reagent (Sigma). These pseudotyped virions do not carry the genetic material of the SARS-COV-2 virus other than Spike, which does not bind efficiently to the murine ACE2 receptor, enabling the study of the in vivo effects of Spike to be studied in the absence of viral infection The next day, the medium was replaced with DMEM10 complete medium, and the cells were incubated at 37° C. in 5% CO₂ for 48 h. The supernatant was then harvested, filtered with 0.22-μm Steriflip filters (EMD, Millipore), and ultracentrifuged at 25,000 rpm for 1.5 h at 4° C. The concentrated supernatant was removed, the pellets (viral particles) were resuspended in cold 1× PBS containing 1% fetal bovine serum, and aliquots were stored at −80° C. in a biosecurity level 3 laboratory. For production of control viral particles not expressing the Spike glycoprotein (BALD), the same procedure was used but with the omission of the pCAGGS SARS-COV-2 Spike vector transfection. HIV ENV pseudotyped viral particles were also produced with the same procedure, using an HIV89.6 ENV dual tropic (X4 and R5) expression vector (NIH AIDS Reagent Program) instead of the Spike expression vector.

### In vivo Administration of SARS-COV-2 Spike PVs

Mice were anaesthetized with isoflurane and placed on electric heating pad. Spike pseudotyped or BALD PVs (control) (100 μl) were slowly injected into the retro-orbital plexus with a BD 0.3-ml insulin syringe attached to a 29-gauge needle. After 3 min, the needle was slowly withdrawn, and the mice were allowed to recover. Since the activity of PVs can be influenced by freeze/thaw cycles, all experiments were done with virions that had been freshly thawed and kept at 37° C. Refrozen virion samples were not used. SARS-COV-2 Spike PVs were administered in 12-week-old to 15-week-old male C57BL/6, Fga^{-/-}, and Fggγ^{390-396A} mice. Experiments in Fga^{-/-} mice were done blinded to the mouse genotype. Experiments in Fggγ^{390-396A} mice were done blinded to the mouse genotype and the type of virions. Mice were randomly assigned to treatment groups in a blinded manner. Genotype and treatment assignment were revealed after image quantification.

### 5B8 Treatment

For pharmacological treatment after SARS-COV-2 Spike PV administration, anti-fibrin antibody 5B8 (Ryu et al. Nat Immunol 19: 1212-1223 (2018)) or an isotype-matched IgG2b (MPC-11, BioXCell) control were administered by retroorbital injection at 30 mg/kg 15 min before injection of Spike PVs to WT mice as described above. Mice were sacrificed at 24 h for histological analysis. Experimenters were blinded to treatment. Treatment assignments were revealed after histologic analysis and image quantification.

### Immunohistochemistry

Lung tissues were cut with a cryostat into 30-um-thick frozen sections for free-floating immunostaining. The following antibodies were used: mouse anti-SARS-COV-2 (COVID-19) Spike antibody (1A9, GeneTex; 1:100), rat anti-mouse CD106 (VCAM-1, catalog no. 553849, BD Pharmingen; 1:100), and rabbit polyclonal anti-fibrinogen (gift from Dr. Jay Degen; 1:500). The tissue sections were washed in PBS and incubated in a blocking and permeabilization buffer consisting of PBS supplemented with 0.2% Triton X-100 and 5% BSA for 1 h at 25° C. For mouse primary antibodies, sections were incubated in M.O.M. mouse IgG blocking reagent diluted in PBS containing 0.2% Triton X-100, and 5% BSA and then with M.O.M. diluent for 5 min at room temperature (M.O.M. (Mouse on Mouse) Immunodetection Kits, Vector Laboratories). Sections were rinsed twice with PBS containing 0.1% Triton X-100 and incubated overnight with primary antibodies at 4° C. All tissue sections were washed with PBS containing 0.1% Triton X-100 and incubated with the following secondary antibodies: goat anti-rabbit Alexa Fluor 488 (1:1000, Thermo Fisher Scientific, A-11008), goat anti-mouse Alexa Fluor 568 (A-110041, Thermo Fisher Scientific; 1:1000), or goat anti-rat Alexa Fluor 647 (A-21247, Thermo Fisher Scientific; 1:1000), and stained with DAPI. Sections were mounted on frosted microscopic slides (Thermo Fisher Scientific), covered with glass coverslips, sealed with ProLong Diamond Antifade Mounting reagent (Thermo Fisher Scientific), and kept at 4° C. until imaging. To assess nonspecific antibody binding, negative control sections were incubated with Isotype-matched, nonspecific mouse, rat, and rabbit antibodies (catalog nos. 08-6599, 31933, and 08-6199, respectively, Thermo Fisher Scientific.

### Confocal Microscopy

Tissue sections were imaged with a laser-scanning confocal microscope (FLUOVIEW FV3000RS “Snow Leopard”), a 60× oil-immersion UPLSAPO objective (NA=1.35), and FV31S-SW software, v 2.3.2.169 (Olympus). Individual channels were captured sequentially with a 405-nm laser and a 430/70 spectral detector for DAPI, a 488-nm laser and a 500/40 spectral detector for Alexa Fluor 488, a 561-nm laser and a 570/620 high-sensitivity detector for Alexa Fluor 568, and a 650-nm laser and a 650/750 high-sensitivity detector (Olympus TruSpectral detector technology) for Alexa Fluor 647. Captured images were processed with Fiji 2.1.0/Image J 1.53c.

### Image Analysis

Immunostained cells were counted with Jupyter Notebook in Python 3. Briefly, an arbitrary threshold was manually set and used for all images in the dataset. The total number of cells per image was estimated with the function peak_local_max). from the open source “skimage” Python image processing library, which returns the coordinates and number of local peaks in an image (see website at scikit-image.org/docs/dev/api/skimage.feature.html#skimage.feature.peak_local_max). Fibrinogen immunoreactivity was quantified with Fiji (ImageJ) as described (Davalos et al., Nat Commun 3: 1227 (2012)). Python image processing was used to colocalize fibrinogen and Spike protein in lung tissues. Briefly, a Jupyter Notebook was written to estimate the amount of fluorescent signal overlap between Spike and fibrinogen in lung tissues. The “Ostu” filter from the “skimage” Python image processing library was used to threshold each image labeled with Spike and fibrinogen (see website at scikitimage.org/docs/0.13.x/api/skimage.filters.html#skimage.filters.threshold_otsu). After thresholding, each set of images was compared, and pixels were compartmentalized in four categories: Spike and fibrinogen overlap, Spike signal only, fibrinogen signal only, and no signal. In each image, the total number of pixels in an image and the number of pixels with signal for Spike only, fibrinogen only, or both were computed for 24 images.

### Gene Expression Analysis

SARS-COV-2 Spike PVs were administered in male C57BL6/J mice as described above. 24 hours after injection, mice were perfused with PBS following isolation of the lungs. A small piece of tissue from each lobe of the lung was dissected, combined, and immediately homogenized in a 1.5-ml Eppendorf tube with buffer RLT (Qiagen) and a pestle (catalog no. 749521-1500, Kimble Chase) on ice. The homogenate was further processed with QIAshredder (Qiagen), and RNA samples were extracted with the RNeasy Mini Kit (Qiagen). RNA concentration was measured with a Nanodrop spectrophotometer (catalog no. 840-274200, Thermo Scientific) and the integrity was determined with an Agilent Bioanalyzer. RNA samples were sent to the Core Center for Musculoskeletal Biology and Medicine at

UCSF, and the gene expression of Mouse Immunology Panel (Codeset: NS_Immunology_Mm_C2269) was determined with a NanoString nCounter machine.

### Network Analysis

Network analysis was performed as described (Mendiola et al. Nat Immunol 21: 513-524 (2020)). The STRING database (Szklarczyk et al. Nucleic Acids Res 49; D605-D612 (2021); Szklarczyk et al. Nucleic Acids Res 45: D362-D368 (2017)) was queried for interactions among the 51 significantly upregulated genes (P value<0.05) after injection of Spike PVs relative to expression after injection of BALD PVs. A subset of 43 genes was connected by high-confidence interactions (score >0.8) and visualized with Cytoscape (Shannon et al. Genome Res 13, 2498-2504 (2003)) Degree was calculated with the built-in Analyzer tool and mapped to node size, while the log2FoldChange was mapped to node fill color with a gradient over the full range of values (0.074-0.723). The network will be made available at NDEx under doi: 10.18119/N9WK60.

### Stereotactic Injection of Fibrinogen and SPIKE

Fibrinogen was stereotactically injected into the brain as described (Ryu et al. Nat Commun 6: 8164 (2015)). Mice were anesthetized with isoflurane and placed in a stereotaxic apparatus (Kopf Instruments). Alexa Fluor 488 human fibrinogen (Thermo Fisher Scientific) was dissolved in 0.1 M sodium bicarbonate (pH 8.3) at 25° C. to a concentration of 1.5 mg/ml as described (Tognatta et al. STAR Protoc 2: 100638 (2021)), mixed with Spike PVs, BALD PVs, or PBS control (1:1 ratio), and incubated at 37° C. for 15 min, 1.5 μl of the mixture was stereotactically injected at 0.3 l /min with a 10-μl Hamilton syringe and a 33-gauge needle into the corpus callosum of C57B1/6 mice (Ryu et al., Nat Commun 6. 8164 (2015)). The mice anesthetized with avertin and transcardially perfused with 4% paraformaldehyde in PBS. The brains were removed, postfixed overnight at 4° C. in 4% paraformaldehyde in PBS, processed with 30% sucrose in PBS, cut into coronal sections 30 μm thick, washed in PBS, and incubated for 10 min with DAPI (Thermo Fisher Scientific; 1:1000) in PBS, and processed for immunohistochemical staining with rabbit anti-allograft inflammatory factor 1 (anti-Iba-1; catalog no. 019-19741, Wako; 1:1,000) as described by Ryu et al. (Nat Commun 6: 8164 (2015)). Images were acquired with an Axioplan II epifluorescence microscope (Zeiss) and Plan-Neofluar objectives (10×0.3 NA). Images of similar anatomical locations were quantified with NIH ImageJ (v. 1.50) by an observer blinded to experimental conditions. Images were acquired and quantified in a blinded manner. Treatment assignment were revealed after image quantification.

### Fibrin Autoantibody Detection in COVID-19 Patient Sera

This experiment was carried out in the BSL-3 facility. Fibrin-coated plates were washed with 1× PBS and blocked with 4 mg/ml mouse serum solution (Molecular Biosciences, dissolved in 5% BSA in PBS) for 1 hour at 25° C. Wells were washed three times for 5 minutes each with wash buffer (0.05% Tween in 1× PBS). Frozen human serum samples were stored at −80° C. with minimal freeze and thaw except for aliquoting purposes until the screen. Once thawed on ice, each sample was gently mixed by pipetting, diluted 1:2 in sample dilution buffer (0.4 mg/ml mouse serum, 0.5% BSA, 0.05% Tween-20 in 1× PBS). Samples were plated in duplicate, incubated for 2 hours at 37° C. on fibrin-coated plates. Wells were washed in wash buffer five times for 5 min each. Fibrin bound human IgG was detected by incubation with mouse anti-human IgG-HRP (1:5000, Invitrogen) for 1 hour at 25° C. in the dark. After thorough washes in wash buffer, HRP substrate (TMB, Millipore) was added, and after adequate colorimetric development wells were neutralized with 1N hydrochloric acid. All samples shown were screened at the same time. Plates were read immediately at 450 nm. Individual well reads were corrected by subtracting secondary background signal.

### Statistical Analysis

All values are reported as mean ±s.e.m. Unless stated otherwise, P values were calculated with one-way or two-way analysis of variance followed Tukey's post hoc test for multiple comparisons or two-tailed Mann-Whitney test for non-normally distributed pairs in GraphPad Prism software 6.0. Sample sizes were determined by prior studies rather than statistical approaches. All mice survived until the end of the study, and all of the data was analyzed. For in vivo studies with Fga^{-/-} mice, only mice, not virions, were randomized and coded for group assignment and data collection. For Fagy 390-3964 mice, both mice and virions were blindly assigned to experimental groups. For the antibody treatment, SB8 and IgG2b were blindly assigned to experiment groups. For the NanoString experiment, virions were blindly assigned to experimental groups. All histological analysis and quantification were done in blinded fashion. No data were excluded. Biochemical studies of the binding of fibrinogen to Spike or Spike PVs were performed in the Akassoglou lab and independently validated in the Greene lab.

For quantification of the fibrin clots by scanning electron microscopy data, at each radius, the difference in log odds of detecting fibers (among all the views in a given image) with the chosen radius under Spike versus control conditions was estimated across all images (log odds ratio) The log odds ratio at each radius was estimated with generalized linear mixed effects models, with the family argument set to binomial and implemented in glmer function in the Ime4 package in R (Douglas et al. J Stat Softw 67: 1-48 (2015)), in which the image source for the observations is modeled as a random effect. P values were corrected for multiple testing with the Holm procedure (Holm, Scandinavian Journal of Statistics 6: 65-70 (1979)) For co-localization of fibrin and Spike, the ratio of the odds that a pixel with signal for Spike would also have signal for fibrinogen to the odds that a pixel with Spike would not have signal for fibrinogen was estimated for each image with Fisher's exact test. For gene expression analysis from NanoString data, the normalized gene expression data from four replicates under each of Spike or BALD PV conditions were log 2 transformed. Up-regulation or down-regulation of expression for each gene was estimated with a one-sided two-sample Welch/test. The resulting P values were corrected for multiple testing with the Benjamini-Hochberg procedure (Benjamini & Hochberg, J R Stat Soc Series B Stat Methodol 57. 289-300 (1995)). All analyses were done in a given number of sections, or cells per lung tissue imaged in vivo, per mouse, and the mean ±s.e.m. was calculated for the reported number (n) of mice per group.

### EXAMPLE 2: DESIGN OF FIBRIN-TARGETING IMMUNOTHERAPY

The C-terminus of the fibrinogen y chain has two different sites at γ400-411 and γ377-395 that are involved in platelet engagement and inflammation respectively. These two sites are distinct domains within the three-dimensional structure of the fibrinogen protein. Peptide 7400-411 is the binding site for the platelet auß3 integrin receptor and is required for platelet aggregation. Peptide γ377-395 is the binding site for the CD11b I-domain of complement receptor 3 (CR3) (also known as CD11b/CD18, Mac-1, α_{M}β2; Ugarova et al. Biochemistry 42, 9365-9373 (2003)) and is required for fibrin-induced activation of microglia and macrophages. The γ377-395 binding site is considered “cryptic” in the soluble fibrinogen molecule and is exposed only upon conversion of fibrinogen to insoluble fibrin.

The inventors hypothesized that monoclonal antibodies against various fibrin/fibrinogen epitopes might block SARS-COV-2 induced thrombosis or SARS-CoV-2 induced inflammation caused by SARS-COV-2.

The inventors had previously prepared antibodies against various fibrin epitopes using the following procedures.

Peptides corresponding to the exact amino acids on the y chain of fibrin that have been shown to be needed for the interaction of fibrin/fibrinogen with Mac-1 were synthesized

(Peptide #1: CGWTVLQKRIDGSL (SEQ ID NO: 4) and
Peptide #2: CKKTTMKIIPFNRLTIG (SEQ ID NO: 2)).

These two peptides were synthesized with N-terminal cysteine residues to allow for conjugation to the carrier protein keyhole limpet hemocyanin (KLH) which promotes a robust antibody response in vivo. Both peptides were used to immunize three mice generating an antibody response in these mice. Preliminary serum screening revealed a strong antibody titer against these peptides and lead to the subsequent generation of hybridomas producing clonal antibodies against these two peptide sequences. The initial screening of 480 hybridoma clones was performed by ELISA against both peptides as well as the carrier protein. The positive clones were expanded and retested to confirm peptide epitope reactivity by ELISA. The final results of this initial screen resulted in 46 clones that were specific to either Peptide #1 or #2. In depth analysis of these ELISA results identified 16 target candidates for further examination. These 16 clones were screened for their ability to block microglial adhesion via the Mac-1 receptor on full length fibrinogen. Tissue culture wells were coated with 50 μg/mL fibrinogen upon which microglia cells (200,000 cells/mL) were plated in the presence of these antibody clones. Wells were washed after 30 minutes and the remaining adherent cells were stained with 0.1% crystal violet. Stained cells were fixed with 1% PFA and solubilized with 0.5% Triton X-100. Five of these clones showed a significant ability, similar to that of a commercially available blocking antibody to Mac-1 (M1/70), to prevent microglial adhesion to fibrinogen as assessed by absorbance measurements at 595 nm.

Clones 1A5, 1D6 and 1E3 recognized the Peptide #1 epitope while clones 4E11 and 5B8 recognize the Peptide #2 epitope. The 5B8 monoclonal antibody has previously been shown by the inventors to inhibit neuroinflammation (Ryu et. Nat Immunol. 19(11): 1212-1223 (November 2018).

The five antibody preparations were further analyzed for their ability to recognize fibrinogen by western blot. All five antibodies recognized fibrinogen's γ chain to a similar degree. To examine whether these antibodies recognized fibrinogen in a dose dependent manner an ELISA was performed on full length coated fibrinogen. All five antibodies were found to specifically bind increasing concentrations of full-length fibrinogen. From these five antibodies three were chosen (IE3, 4E11 and 5B8, having greater than 50% inhibition of Mac-1 binding to the fibrin or fibrinogen γC domain when measured by shift in absorbance) for isolation and large-scale purification.

Antibodies 5B8, 4E11, and 4F1 had the highest selectivity and specificity for the γ377-395 region of fibrinogen. All antibodies against cryptic epitopes bound with higher affinity to fibrin than to fibrinogen. Conversion of fibrinogen into fibrin exposes amino acids 377-395 in the fibrinogen chain. Hence, this region may be more accessible in fibrin than in fibrinogen. Among antibodies targeting γ377-395, the 5B8 antibodies bound fibrin to the greatest degree with minimal binding to soluble fibrinogen. Competitive binding assays showed that 5B8 bound to human and mouse γ377-395 peptides, but not γ190-202 peptide. The 5B8 antibodies also inhibited binding of the CD11b I-domain to fibrin, indicating that the 5B8 antibodies interfere with the ligand-receptor interaction.

### EXAMPLE 3: ANIMAL MODEL OF SARS-COV-2 EFFECTS

Mice were selected as an animal model for evaluation of the effects of SARS-CoV-2 infection on various organ systems.

Pseudotyped SARS-COV-2 viral particles encoding wild type spike protein were formulated for administrations to the mice. In addition, ‘bald’ virion particles that did not encode spike proteins (mock) were formulated to serve as a negative control.

Pseudotyped SARS-COV-2 Spike protein virions were produced by using an HIV Env-deficient packaging vector lacking its natural Env gene (HIV-1 NL4-3 ΔEnv EGFP Reporter Vector) with a viral packaging system. An example of a sequence for a plasmid/expression vector for SARS-COV-2 Spike protein is the pCAGGS vector with the NR-52310 Spike protein insert provided by beiresources.org. ‘BALD’ virions that do not express the SARS-COV-2 Spike protein or the HIV Env protein were generated to serve as a negative control.

The pseudotyped SARS-COV-2 virions encoding wild type spike protein and the bald viral particles were administered to the mice and the pathological effects on the animals were monitored.

SARS-COV-2 infection can negatively affect the brain, gut, kidneys, vascular system, and lungs of the mice. In the brain, neuroinflammation was prevalent, the blood brain barrier was disrupted, and fibrin deposition was visible. The lungs also exhibited inflammation and fibrin deposition, as well as oxidative stress.

After administration of the pseudotyped SARS-COV-2 Spike protein virions, a preparation of the 5B8 anti-fibrin antibodies was administered to an experimental group of mice. As a control, some mice administered the pseudotyped SARS-COV-2 Spike protein virions were administered a non-reactive isotype-matched preparation of IgG2b antibodies.

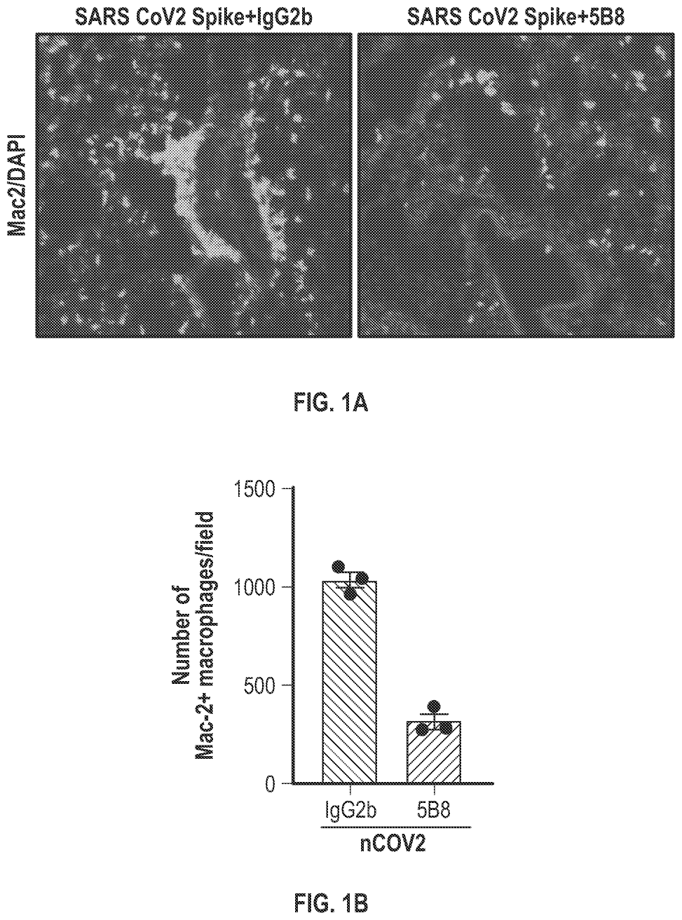

As illustrated in FIG. 1A, the 5B8 antibodies significantly reduced inflammation in the lungs of mice administered the pseudotyped SARS-COV-2 Spike protein virions compared to the lungs of control mice that received the non-reactive IgG2b antibodies. FIG. 1B shows that the number per field of Mac-2⁺ macrophages was significantly reduced in the lungs of mice that received the 5B8 antibodies after receiving the pseudotyped SARS-COV-2 spike protein virions compared to the control mice that received the non-reactive IgG2b antibodies.

### EXAMPLE 4: FIBRIN AND FIBRINOGEN BIND TO SARS-COV-2 SPIKE PROTEIN

This Example illustrates that the SARS-COV-2 spike protein binds fibrin and fibrinogen.

Varying amounts of SARS-COV-2 spike protein were incubated on fibrin- or fibrinogen- coated plates, the plates were washed, and the quantity of bound spike protein was detected by use of a labeled anti-spike antibody.

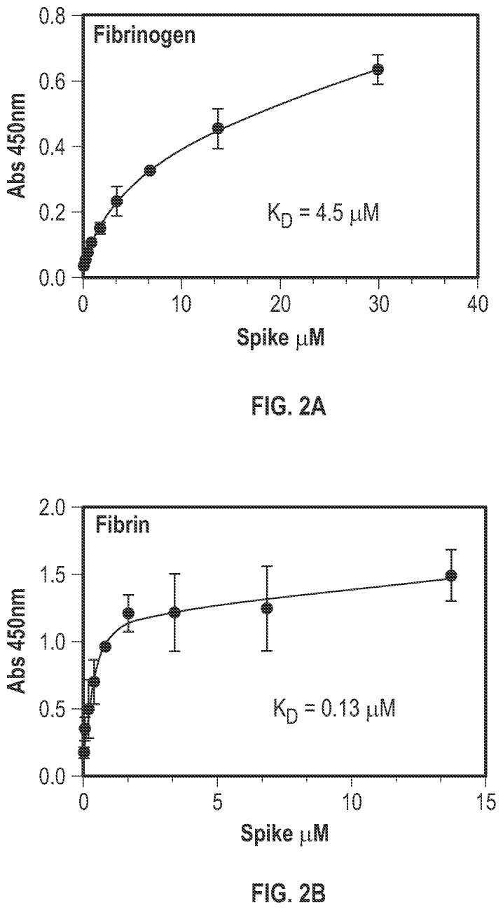

As shown in FIGS. 2A-2B, the amount of spike protein bound to fibrin and fibrinogen is directly proportional to the amount of spike protein incubated on the fibrin-coated plates.

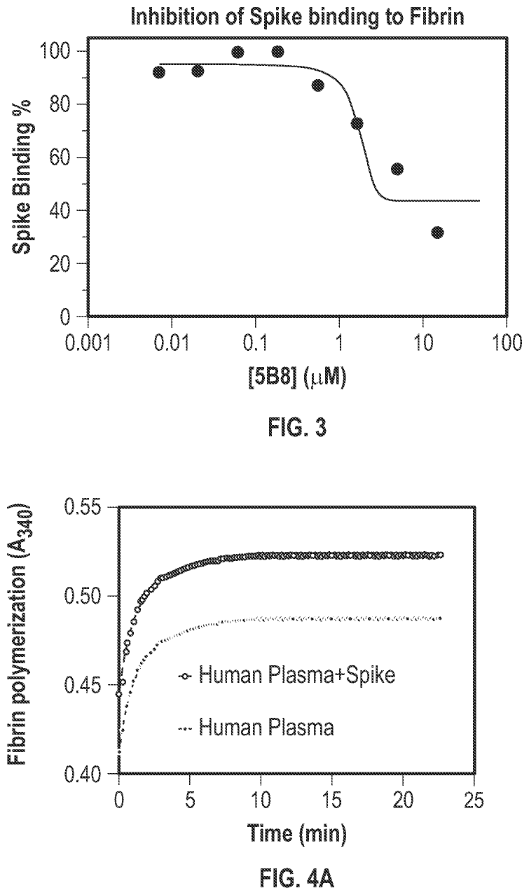

Moreover, when varying amounts of the 5B8 anti-fibrin antibodies are incubated in solution with a set amount of SARS-COV-2 spike protein on the fibrin-coated plates, the amount of SARS-COV-2 spike protein bound to the fibrin is significantly reduced (FIG. 3).

Hence, 5B8 anti-fibrin antibodies inhibit binding of the SARS-COV-2 spike protein to fibrin.

### EXAMPLE 5: SPIKE BINDING SITES ON FIBRINOGEN

As illustrated herein, hypercoagulability in COVID-19 patients has features distinct from those of other inflammatory diseases and the inventors have shown that SARS-COV-2 directly affects the structural and functional properties of blood clots.

FIG. 4A further illustrates that incubation of SARS-COV-2 recombinant trimeric spike protein (Spike) with healthy donor plasma increased fibrin polymerization. Spike strikingly altered the fibrin clot structure resulting in thinner fibers with a rough appearance and increased clot density as shown by scanning electron microscopy (SEM) (FIG. 4B). Therefore, the SARS-COV-2 Spike protein has direct effects on fibrin clot architecture.

Consistent with these structural changes, a solid-phase binding assay revealed binding of Spike to both fibrinogen and fibrin (K_{d}5.3 μM and 0.4 μM, respectively) (FIG. 4C). Fibrinogen was immunoprecipitated with full-length recombinant trimeric Spike (FIG. 4D). Studies with Spike deletion mutants identified an interaction with the S2 domain of Spike (amino acids 686-1273; FIG. 4D).

Fibrinogen is a 340 kDa protein consisting of three pairs of polypeptide chains Aα, Bγ, and γ (Doolittle et al., Ann N Y Acad Sci 936: 31-43 (2001)). To identify Spike binding regions on fibrinogen, a custom fibrinogen peptide array was generated consisting of 390 15-mer peptides overlapping by eleven amino acids and spanning the fibrinogen Aα, Bβ, and γ chains (FIG. 4E). Hybridization with His-tagged trimeric Spike identified three binding sites in the Bβ and γ fibrinogen chains, namely the Bβ119-129 (YLLKDLWQKRQ, SEQ ID NO:41), γ163-181 (QSGLYFIKPLKANQQFLVY; SEQ ID NO:42) and γ7364-395 (DNGIIWATWKTRWYSMKKTTMKIIPFNRLTIG; SEQ ID NO:43) fibrinogen peptides (FIG. 4E). The Bβ119-129 peptide contains cleavage sites for the fibrinolytic serine protease plasmin (Lijnen et al., Ann N Y Acad Sci 936: 226-236 (2001)). Spike bound to the γ364-395 peptide, which encompasses the γ377-395 cryptic fibrinogen binding site to complement receptor 3 that activates innate immune responses (Davalos & Akassoglou, Semin Immunopathol 34: 43-62 (2012); Ugarova et al., Biochemistry 42:

9365-9373 (2003)). Spike also bound to the y163-181 peptide, whose function is unknown. Mapping of the Spike binding peptides onto the crystal structure of fibrinogen revealed proximity of the γ163-181 and γ377-395 peptides, suggesting that a 3D conformational cpitope in the carboxy-terminal γ-chain of fibrinogen (γC domain) is involved in fibrinogen binding to Spike.

Spike binds to fibrinogen sites involved in regulation of plasmin cleavage and binding to complement receptor 3. The inventors therefore decided to test whether Spike binding interferes with the fibrin degradation and with the inflammatory properties of fibrin. Incubation of Spike with fibrin delayed plasmin degradation of both the γ-chain and the γ-γ dimer (FIG. 4F), indicating that Spike delays fibrinolysis.

This finding is consistent with dense fibrin clots composed of thin fibers that the inventors identified and the presence of fibrinolysis-resistant blood clots in COVID-19 patients (Mitrovic et al., Platelets 32: 690-696 (2021)). Dense fibrin clots with thin fibers resistant to lysis are also observed in thromboembolic diseases (Undas & Ariens, Arterioscler Thromb Vasc Biol 31, e88-99 (2011)).

Fibrin is deposited locally at sites of vascular damage and is a potent proinflammatory activator and a key inducer of oxidative stress (Davalos & Akassoglou, Semin Immunopathol 34, 43-62 (2012); Ryu et al., Nat Immunol 19, 1212-1223 (2018)). Strikingly, Spike increased fibrin-induced release of reactive oxygen species (ROS) in a concentration-dependent manner in bone marrow-derived macrophages (BMDMs), while Spike alone did not have an effect (FIG. 4G). These results indicate that the Spike protein has a role as an enhancer of fibrin-induced inflammation at sites of vascular damage.

Overall, these results reveal that the SARS-COV-2 Spike protein has an unanticipated role as a fibrinogen binding protein that can accelerate the formation of abnormal clots with altered structures and increased inflammatory activities.

In COVID-19 patients, fibrin is deposited in the air spaces and lung parenchyma and is associated with inflammation (Fox et al., Lancet Respir Med 8: 681-686 (2020)). The inventors developed an experimental platform to study the interplay between fibrin and SARS-COV-2 Spike in vivo by injecting mice with HIV virions pseudotyped with SARS-COV-2 trimeric Spike glycoprotein (Spike PVs) (FIG. S3), enabling the study of the in vivo effects of Spike independent of active viral replication. The pseudotyped SARS-COV-2 Spike protein virions were produced by using an HIV Env-deficient packaging vector lacking its natural Env gene (HIV-1 NL4-3 ΔEnv EGFP Reporter Vector) with a viral packaging. As shown in FIG. 4H, the HIV-1 NL4-3 ΔEnv pro-viral DNA vector was co-transfected with the SARS-CoV-2 trimeric Spike glycoprotein expression vector into 293T cells Forty-eight hours after transfection, supernatant was harvested and Spike pseudotyped virions (PVs) were pelleted by ultracentrifugation and collected.

Intravenous administration of Spike PVs in wild-type (WT) mice induced extensive fibrin deposition in the lung (FIG. 5A). Double immunofluorescence staining for fibrin and Spike PVs revealed strong overlap of Spike and fibrin deposits (FIG. 5B).

Fibrin deposition was associated with activated endothelium in the lung. Gene expression analysis revealed increased expression of endothelial and inflammatory markers in Spike PV-injected mice compared to mice injected with control BALD PVs (FIG. 5C), consistent with findings of SARS-COV-2 toxicity to endothelial cells (Varga et al., Lancet 395, 1417-1418 (2020))

Fibrin activates macrophages and induces oxidative stress through nicotinamide adenine dinucleotide phosphate (NADPH) oxidase (Davalos & Akassoglou, Semin Immunopathol 34, 43-62 (2012); Ryu et al., Nat Immunol 19, 1212-1223 (2018)), which is linked to severe disease and thrombotic events in COVID-19 patients (Violi et al., Redox Biol 36, 101655 (2020)). In WT mice, Spike-PVs activated macrophages and increased expression of the gp-91-phox subunit of NADPH oxidase in the lung of WT mice indicating the generation of an oxidative stress response (FIG. 5D). In contrast, control BALD PVs or PVs expressing the Env protein from the HIV-1(HIV-1 PVs) did not induce these effects (FIG. SD), indicating that lung pathology was specific for SARS-COV-2 Spike. Mice genetically-deficient in fibrinogen (Fga^{-/-}mice), which express all other blood proteins except fibrinogen and are protected from autoimmune and inflammatory conditions (Davalos & Akassoglou, Semin Immimopathol 34, 43-62 (2012)), did not exhibit lung pathology following Spike PV challenge (FIG. SE). These results reveal a Spike-fibrinogen-dependent mechanism of clot formation that generates strong inflammatory and oxidative stress responses.

Fibrinogen is causally linked to the activation of macrophages and microglia in autoimmune and inflammatory diseases in the brain and periphery (Davalos & Akassoglou, Semin Immunopathol 34, 43-62 (2012); Petersen, Ryu, & Akassoglou, Nat Rev Neurosci 19, 283-301 (2018)). Fibrin is a driver of microglia-induced cognitive dysfunction (Merlini et al., Neuron 101, 1099-1108 (2019)) and is associated with perivascular-activated microglia and macrophages in brains of COVID-19 patients even without signs of infection (Lee et al., N Engl J Med 384, 481-483 (2021)). Stereotactic injection of fibrinogen into the brains of WT mice is a model of fibrinogen-induced encephalomyelitis (Petersen, Ryu, & Akassoglou, Nat Rev Neurosci 19, 283-301 (2018))). Co-injection of Spike PVs and fibrinogen into the brains of WT mice significantly increased fibrin-induced microglia activation (FIG. 6A), indicating that Spike enhances the inflammatory function of fibrin in vivo. Like recombinant Spike (FIGS. 4D, 4G), Spike PVs co-immunoprecipitated with fibrinogen and increased fibrin-induced oxidative stress in BMDMs.

Conversion of fibrinogen to fibrin exposes the cryptic inflammatory y377-395 epitope in the fibrinogen y-chain. Genetic or pharmacologic targeting of this epitope has potent therapeutic effects in autoimmune and inflammatory diseases (Davalos & Akassoglou, Semin Immunopathol 34, 43-62 (2012); Ryu et al., Nat Immunol 19, 1212-1223 (2018); Flick et al., J Clin Invest 113, 1596-1606 (2004); Adams et al., J Exp Med 204, 571-582 (2007)).

Alanine scanning mutagenesis was used to locate where Spike interacts with fibrin/fibrinogen. A series of mutant fibrin peptides were evaluated to determine the Spike protein binding site. Sequences for some of the peptides evaluated are shown below.

(SEQ ID NO: 44)
YSMKKTTMKIIPENRLTIG
(SEQ ID NO: 45)
ASMKKTTMKIIPENRLTIG
(SEQ ID NO: 46)
YAMKKTTMKIIPENRLTIG
(SEQ ID NO: 47)
YSAKKTTMKIIPFNRLTIG
(SEQ ID NO: 48)
YSMAKTTMKIIPENRLTIG
(SEQ ID NO: 49)
YSMKATTMKIIPENRLTIG
(SEQ ID NO: 50)
YSMKKATMKIIPENRLTIG
(SEQ ID NO: 51)
YSMKKTAMKIIPFNRLTIG
(SEQ ID NO: 52)
YSMKKTTAKIIPFNRLTIG
(SEQ ID NO: 53)
YSMKKTTMAIIPENRLTIG
(SEQ ID NO: 54)
YSMKKTTMKAIPENRLTIG
(SEQ ID NO: 55)
YSMKKTTMKIAPFNRLTIG
(SEQ ID NO: 56)
YSMKKTTMKIIAFNRLTIG
(SEQ ID NO: 57)
YSMKKTTMKIIPANRLTIG
(SEQ ID NO: 58)
YSMKKTTMKIIPFARLTIG
(SEQ ID NO: 59)
YSMKKTTMKIIPENALTIG
(SEQ ID NO: 60)
YSMKKTTMKIIPFNRATIG
(SEQ ID NO: 61)
YSMKKTTMKIIPENRLAIG
(SEQ ID NO: 62)
YSMKKTTMKIIPENRLTAG
(SEQ ID NO: 63)
YSMKKTTMKIIPFNRLTIA

As shown in FIG. 6B, fibrin/fibrinogen sequences at amino acid positions 386-394 in the C-terminus of the γ377-395 epitope are involved in spike binding to fibrin/fibrinogen. Residues with low signal intensity upon Ala substitution (Bold residues) required for binding included the IIPFXRLXI sequence (SEQ ID NO:64) shown at the top.

FIG. 6C illustrates that genetic targeting of the fibrin γ377-395 epitope in Fggγ390-3964 mice, in which fibrinogen retains normal clotting function but lacks the γ390-396-binding motif, rescued from macrophage activation and oxidative stress in the lung after Spike PV administration. Because the fibrin γ377-395 peptide is a binding site for both Spike (this study) and complement receptor inhibition of this epitope may reduce their interactions with fibrin.

These findings reveal a previously unknown interaction between SARS-COV-2 Spike protein and fibrin γ377-395 epitope that promotes innate immune activation.

### EXAMPLE 6: FIBRIN AUTOANTIBODIES ARE ABUNDANT IN COVID-19 PATIENTS

A surge of autoantibody production against diverse immune targets have been detected in COVID-19 patients (Wang et al., Nature 595, 283-288 (2021)). To determine whether COVID-19 patients develop autoantibodies against abnormal blood clots, the inventors tested autoantibody responses to fibrin. Autoantibodies against fibrin epitopes would be potentially missed by the inherent limitations of phage and yeast library screens to produce post-translationally modified insoluble fibrin polymer. To overcome this challenge, the inventors developed a fibrin autoantibody discovery platform optimized for screening patient samples. longitudinally collected serum samples ranging from acute to convalescent disease stages from 54 COVID-19 asymptomatic, mild, and severe disease patients requiring admission to the intensive care units were tested. The characteristics of the COVID-19 patients are shown in Table 1.

TABLE 1
Characteristics of COVID-19 and control patients
included in the fibrin autoantibody screen

| | | | | | |
|---|---|---|---|---|---|
| | Severe | Mild | Asymptomatic | Healthy | Non-COVID |
| Patient | COVID-19 | COVID-19 | COVID-19 | Control | Respiratory Illness |
| Characteristics | n = 19 | n = 20 | n = 15 | n = 9 | n = 8 |

Age, y

| | | | | | |
|---|---|---|---|---|---|
| Median | 60 | 44 | 53 | 37 | 61 |
| Mean (SD) | 60.05 | 47.74 | 54.9 | 40.4 | 64.9 |
| | (13.9) | (12.6) | (14.6) | (9) | (15.8) |

Sex

| | | | | | |
|---|---|---|---|---|---|
| Male | 16 | 14 | 8 | 4 | 8 |
| Female | 3 | 6 | 7 | 5 | 0 |

Level of Care

| | | | | | |
|---|---|---|---|---|---|
| Mechanical | 19 | 0 | 0 | 0 | — |
| ventilation | | | | | |

Mortality

| | | | | |
|---|---|---|---|---|
| 3 | 0 | 0 | 0 | — |

Medical History

| | | | | | |
|---|---|---|---|---|---|
| Hypertension | 14 | 7 | 6 | 0 | — |
| Type 2 diabetes | 10 | 2 | 4 | 0 | — |
| Obesity | 8 | 3 | 3 | 0 | — |
| Coronary artery | 1 | 1 | 0 | 0 | — |
| disease | | | | | |
| Congestive | 2 | 1 | 1 | 0 | — |
| heart failure | | | | | |
| Stroke | 1 | 0 | 0 | 0 | — |
| Chronic kidney | 1 | 0 | 1 | 0 | — |
| disease | | | | | |
| Chronic liver | 0 | 0 | 1 | 0 | — |
| disease | | | | | |
| COPD | 1 | 0 | 1 | 0 | — |
| Asthma | 0 | 0 | 1 | 0 | — |
| Organ | 0 | 1 | 0 | 0 | — |
| transplant | | | | | |
| Immuno- | 0 | 1 | 0 | 0 | — |
| compromised | | | | | |
| HIV | 0 | 0 | 1 | 0 | — |
| None | 3 | 8 | 4 | 0 | — |

Symptoms

| | | | | | |
|---|---|---|---|---|---|
| Fever | 13 | 13 | 0 | 0 | — |
| Cough | 14 | 8 | 0 | 0 | — |
| Shortness of | 15 | 5 | 0 | 0 | — |
| breath | | | | | |
| Sore throat | 3 | 5 | 0 | 0 | — |
| Chest pain | 2 | 3 | 0 | 0 | — |
| Loss of | 0 | 1 | 0 | 0 | — |
| taste/smell | | | | | |
| Headache | 3 | 4 | 0 | 0 | — |
| Myalgia | 4 | 6 | 0 | 0 | — |
| Chills | 3 | 6 | 0 | 0 | — |
| Diarrhea | 3 | 3 | 0 | 0 | — |

As shown in FIGS. 7A-7B, fibrin autoantibodies were abundant in all three groups of COVID-19 patients and persisted during the convalescent stage but were scarce in healthy donor controls or in subjects with non-COVID respiratory illnesses. Blockade of the thromboinflammation cascade following Spike and fibrinogen/fibrin interaction is an attractive therapeutic target. Based on the inventors' genetic rescue results implicating a causal role for the y377-395 epitope, the inventors tested the effects of 5B8, a monoclonal antibody generated against the fibrin γ377-395 epitope (Ryu et al., Nat Immunol 19, 1212-1223 (2018)). This selective antibody-based approach suppresses fibrin-induced inflammation without altering normal hemostasis (Ryu et al. (2018)).

As shown in FIG. 7C, the SB8 antibody rescued the enhanced inflammatory effects induced by Spike in fibrin-treated BMDMs, indicating that pharmacologic blockade of the fibrin γ377-395 epitope inhibits the deleterious effects of SARS-CoV-2 Spike as an enhancer of thromboinflammation.

Strikingly, the 5B8 antibody reduced macrophage activation and oxidative stress in the lungs of Spike PV-treated WT mice compared to isotype IgG2b-treated controls (FIGS. 7D-7E). Collectively, these results show that anti-fibrin autoimmune responses occur in COVID-19 patients. These result also demonstrate that 5B8 antibodies provide a potent protective effect against thromboinflammation.

In summary, SARS-COV-2 Spike protein enhances the formation of highly inflammatory clots that are neutralized by fibrin-targeting monoclonal antibodies such as the 5B6 antibodies. The data described herein shed a new light on the enigmatic coagulopathy found in COVID-19, revealing a causal role for fibrinogen in thromboinflammation that is even independent of active viral replication.

The high incidence of clotting complications in COVID-19 has been attributed to systemic inflammation (Merad & Martin, Nat Rev Immunol 20, 355-362 (2020)), vascular damage including abnormal levels of circulating coagulation proteins (Tang et al. Thromb Haemost 18, 844-847 (2020); Ackermann et al., N Engl J Med 383, 120-128 (2020)), genetic susceptibility to tissue factor and complement genes (Ramlall et al., Nat Med 26, 1609-1615 (2020)), and prothrombotic autoantibodies (Zuo et al., Sei Transl Med 12, (2020)). However, the data shown herein demonstrate that coagulopathy is not merely a consequence of inflammation. Rather, the interaction of SARS-COV-2 Spike with fibrinogen and fibrin results in abnormal blood clot formation that in turn drives inflammation.

Identification of SARS-COV-2 Spike protein as a fibrinogen binding partner provides a mechanistic basis for the formation of abnormal clots with enhanced inflammatory properties. This mechanism might be in play at sites of local fibrin deposition and microvascular injury perpetuating a hypercoagulable and inflammatory state as reported in COVID-19 patients (Page, R. A. S. Ariens, Thromb Res 200, 1-8 (2021)) that could be critical during acute infection, as well as in Post-Acute Sequelae of SARS COV-2 infection (PASC).

Fibrin is locally deposited in brain and other organs of COVID-19 patients. Thus, fibrin immunotherapy may represent a novel strategy for reducing thromboinflammation in systemic and neurologic manifestations of COVID-19. Because, as shown herein, the anti-fibrin antibody 5B8 has protective effects and protective autoantibodies targeting fibrin can be an effective strategy against COVID-19.

### EXAMPLE 7: ANTI-FIBRIN(OGEN) ANTIBODIES INHIBIT SPIKE VIRION BINDING

This Example illustrates that anti-fibrin(ogen) antibodies can inhibit or prevent pseudotyped SARS-COV-2 Spike protein expressing virions from binding and accumulating in lung tissues.

Mice (6 per group) were intravenously administered anti-Fibrin(ogen) 5B8 antibodies (30 mg/kg) or IgG2b antibodies (30 mg/kg; control). Twenty-four hours after antibody administration SARS-COV-2 Spike pseudovirions were injected into the mice. Lung tissues were collected and sections were stained with 4′,6-diamidino-2-phenylindole (DAPI; blue) as well as either labeled anti-spike antibodies (bright red) or labeled anti-Fibrin(ogen) antibodies (bright green). The quantities of SARS-CoV-2 Spike protein and fibrin(ogen) were determined by detecting the signals from the labeled antibodies from multiple microscopic fields in each of the six mice conditions.

FIG. 8A shows images of the lung sections, demonstrating that treatment with 5B8 antibody, but not control IgG2b antibody, blocks fibrin and Spike co-deposition in the lungs of mice injected with Spike pseudotyped virions.

FIG. 8B graphically illustrates the quantity of SARS-COV-2 Spike protein when the anti-Fibrin(ogen) 5B8 antibodies or the control IgG antibodies were administered. As shown, when the anti-Fibrin(ogen) 5B8 antibodies were administered, little or no SARS-COV-2 Spike protein was deposited in the lung tissues.

FIG. 8C graphically illustrates the quantity of Fibrin(ogen) when the anti-Fibrin(ogen) 5B8 antibodies or the control IgG antibodies were administered. As shown, when the anti-Fibrin(ogen) 5B8 antibodies were administered, little or no fibrin(ogen) was deposited in the lung tissues.

These findings show that not only do 5B8 anti-Fibrin(gen) antibodies exert anti-inflammatory effects but they also prevent fibrin deposition, which is part of the clotting process.

### REFERENCES

1. N. Tang, D. Li, X. Wang, Z. Sun, J Thromb Haemost 18, 844-847 (2020).
2. Z. Al-Aly, Y. Xie, B. Bowe, Nature 594, 259-264 (2021).
3. M. Merad, J. C. Martin, Nat Rev Immunol 20, 355-362 (2020).
4. E. G. Bouck et al., Arterioscler Thromb Vasc Biol 41, 401-414 (2021).
5. M. Mitrovic et al., Platelets 32, 690-696 (2021).
6. A. E. Merkler et al., JAMA Neurol, (2020).
7. A. Nalbandian et al., Nat Med 27, 601-615 (2021).
8. S. E. Fox et al., Lancet Respir Med 8, 681-686 (2020).
9. T. M. Tu et al., JAMA Netw Open 4, e217498 (2021).
10. R. F. Doolittle, Z. Yang, I. Mochalkin, Ann N Y Acad Sci 936, 31-43 (2001).
11. D. Davalos, K. Akassoglou, Semin Immunopathol 34, 43-62 (2012).
12. M. H. Lee et al., N Engl J Med 384, 481-483 (2021).
13. K. T. Thakur et al., Brain, (2021).
14. E. M. Page, R. A. S. Ariens, Thromb Res 200, 1-8 (2021).
15. H. R. Lijnen, Ann N Y Acad Sci 936, 226-236 (2001).
16. T. P. Ugarova et al., Biochemistry 42, 9365-9373 (2003).
17. A. Undas, R. A. Ariens, Arterioscler Thromb Vasc Biol 31, e88-99 (2011).
18. J. K. Ryu et al., Nat Immunol 19, 1212-1223 (2018).
19. Z. Varga et al., Lancet 395, 1417-1418 (2020).
20. F. Violi et al., Redox Biol 36, 101655 (2020).
21. M. A. Petersen, J. K. Ryu, K. Akassoglou, Nat Rev Neurosci 19, 283-301 (2018).
22. M. Merlini et al., Neuron 101, 1099-1108 (2019).
23. M. J. Flick et al., J Clin Invest 113, 1596-1606 (2004).
24. R. A. Adams et al., J Exp Med 204, 571-582 (2007).
25. E. Y. Wang et al., Nature 595, 283-288 (2021).
26. M. Ackermann et al., N Engl J Med 383, 120-128 (2020).
27. V. Ramlall et al., Nat Med 26, 1609-1615 (2020).
28. Y. Zuo et al., Sci Transl Med 12, (2020).
29. P. Bastard et al., Science 370, (2020).
30. T. T. Suh et al., Genes Dev 9, 2020-2033 (1995).
31. M. J. Flick et al., J Clin Invest 113, 1596-1606 (2004).
32. D. Stadlbauer et al., Curr Protoc Microbiol 57, e100 (2020).
33. J. K. Ryu et al., Nat Immimol 19, 1212-1223 (2018).
35. N. A. Hotaling et al. Biomaterials 61, 327-338 (2015).
36. E. F. Pettersen et al., J Comput Chem 25, 1605-1612 (2004).
37. A. S. Mendiola et al., Nat Imnumol 21, 513-524 (2020).
38. D. Davalos et al., Nat Commun 3, 1227 (2012).
39. D. Szklarczyk et al., Nucleic Acids Res 49, D605-D612 (2021).
40. D. Szklarczyk et al., Nucleic Acids Res 45, D362-D368 (2017).
41. P. Shannon et al., Genome Res 13, 2498-2504 (2003).

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following statements are intended to describe and summarize various embodiments of the invention according to the foregoing description in the specification.

### STATEMENTS

1. A method comprising administering a composition comprising anti-fibrin and/or anti-fibrinogen antibodies to a subject infected with coronavirus, including SARS-COV-2 or SARS-COV-1.
2. The method of statement 1, further comprising administering an antibody that binds to a Coronavirus spike protein to the subject.
3. The method of statement 1 or 2, wherein the composition further comprises an antibody that binds SARS-COV-2 or SARS-COV-1 spike protein to the subject.
4. The method of any one of statements 1-3, which reduces the symptoms of SARS-COV-2 or SARS-COV-1 infection compared to a control subject who did not receive the composition.
5. The method of any one of statements 1-4, which reduces inflammation, oxidative stress, or a combination thereof in the subject compared to a control subject who did not receive the composition.
6. The method of any one of statements 1-5, which reduces inflammation, oxidative stress, or a combination thereof in tissues with fibrin deposition compared to tissues of a control subject who did not receive the composition.
7. The method of any one of statements 1-6, which reduces inflammation, oxidative stress, or a combination thereof in the subject's brain, gut, kidneys, vascular system, lungs, or a combination thereof compared to a control subject who did not receive the composition.
8. The method of any one of statements 1-7, which reduces SARS-COV-2 virus and/or or SARS-COV-1 virus binding to fibrin or fibrinogen compared to SARS-COV-2 virus and/or SARS-COV-1 virus binding to fibrin or fibrinogen of a control subject who did not receive the composition.
9. The method of any one of statements 1-8, which reduces SARS-COV-2 and/or SARS-COV-1 spike protein binding to fibrin or fibrinogen compared to SARS-CoV-2 and/or SARS-COV-1 spike protein binding to fibrin or fibrinogen of a control subject who did not receive the composition.
10. The method of any one of statements 1-9, which reduces Mac-1 protein binding to fibrin or fibrinogen compared to Mac-1 binding to fibrin or fibrinogen of a control subject who did not receive the composition.
11. The method of any one of statements 1-10, comprising greater than 50% inhibition of SARS-COV-2 and/or SARS-COV-1 spike protein binding, SARS-CoV-2 and/or SARS-COV-1 viral particle binding, or Mac-1 binding to the fibrin or fibrinogen, compared to SARS-COV-2 and/or SARS-COV-1 spike protein binding, SARS-COV-2 and/or SARS-COV-1 viral particle binding, or Mac-1 binding to fibrin or fibrinogen of a control subject who did not receive the composition.
12. The method of statement 11, wherein SARS-COV-2 and/or SARS-COV-1 spike protein, SARS-COV-2 and/or SARS-COV-1 viral particle, or Mac-1 binding to the fibrin or fibrinogen γC domain is inhibited compared to such binding to the fibrin or fibrinogen γC domain of SARS-COV-2 and/or SARS-CoV-1 spike protein, SARS-COV-2 and/or SARS-COV-1 viral particle, or Mac-1 from a control subject who did not receive the composition.
13. The method of any one of statements 1-12, which reduces SARS-COV-2 viral entry and/or SARS-COV-1 viral entry into cells.
14. The method of any one of statements 1-13, which reduces SARS-COV-2 viral entry into cells and/or SARS-COV-1 viral entry into cells by at least 25%, or at least 30%, or at least 40%, or at least 50%, or at least 60%, or at least 70%, or at least 80%, or at least 90%, or at least 95%, or at least 98%.
15. The method of any one of statements 1-14, which reduces fibrin deposition in tissues.
16. The method of any one of statements 1-15, which reduces fibrin deposition in tissues by at least 25%, or at least 30%, or at least 40%, or at least 50%, or at least 60%, or at least 70%, or at least 80%, or at least 90%, or at least 95%, or at least 98%.
17. The method of any one of statements 1-16, further comprising administering an anti-spike antibody preparation to the subject.
18. The method of any one of statements 1-17, wherein the anti-fibrin antibodies or anti-fibrinogen antibodies or anti-spike antibodies are human antibodies or humanized antibodies.
19. The method of any one of statements 1-18, wherein the anti-fibrin antibodies or anti-fibrinogen antibodies bind to an epitope with peptide sequence SEQ ID NO:2, Bβ119-129 (YLLKDLWQKRQ, SEQ ID NO:41), γ163-181 (QSGLYFIKPLKANQQFLVY; SEQ ID NO:42), γ364-395 (DNGIIWATWKTRWYSMKKTTMKIIPFNRLTIG; SEQ ID NO:43), IIPFXRLXI (SEQ ID NO:64), or a combination thereof.
20. The method of any one of statements 1-19, wherein the anti-fibrin antibodies or anti-fibrinogen antibodies comprise a CDR region with a sequence comprising SEQ ID NO:6-8, 10-12, or combination of CDR regions with sequences comprising SEQ ID NO:6-8, 10, 11, and 12.
21. The method of any one of statements 1-20, wherein the SARS-COV-2 or SARS-Co-V-1 is infectious SARS-COV-2 or SARS-COV-1.
22. The method of any one of statements 1-21, wherein the composition is administered at a time while SARS-COV-2 or SARS-COV-1 viral replication is occurring in the subject.
23. The method of any one of statements 1-22, wherein the SARS-COV-2 is non-infectious SARS-COV-2 or non-infectious SARS-COV-1.
24. The method of any one of statements 1-23, wherein the composition is administered at a time after SARS-COV-2 or SARS-COV-1 viral replication in the subject is no longer replicating or is not detected.
25. A composition comprising one or more antibodies, small molecules, polypeptides, or a combination thereof, wherein at least one of the antibodies, small molecules, or polypeptides binds to a fibrinogen or fibrin epitope.
26. The composition of statement 25, wherein the fibrinogen or fibrin epitope comprises peptide sequence SEQ ID NO:2, Bβ119-129 (YLLKDLWQKRQ. SEQ ID NO:41), γ163-181 (QSGLYFIKPLKANQQFLVY; SEQ ID NO:42), γ364-395 (DNGIIWATWKTRWYSMKKTTMKIIPFNRLTIG; SEQ ID NO:43), IIPFXRLXI (SEQ ID NO:64), or a combination thereof.
27. The composition of statement 25 or 26, wherein at least one of the antibodies is a human or humanized antibody.
28. The composition of statement 25, 26 , or 27, wherein at least one of the antibodies comprises a CDR region with a sequence comprising SEQ ID NO:6-8, 10-12, or combination of CDR regions with sequences comprising SEQ ID NO:6-8, 10, 11, and 12.
29. The composition of any one of statements 25-28, wherein one or more of the antibodies, small molecules, polypeptides, or a combination thereof, are present the composition in an amount sufficient to reduce symptoms of SARS-CoV-2 infection or SARS-COV-1.
30. The composition of any one of statements 25-29, wherein one or more of the antibodies, small molecules, polypeptides, or a combination thereof, are present the composition in an amount sufficient to reduce brain, gut, kidneys, vascular system, or lung inflammation.
31. The composition of any one of statements 25-30, wherein one or more of the antibodies, small molecules, polypeptides, or a combination thereof, are present the composition in an amount sufficient to reduce SARS-COV-2 or SARS-COV-1 virus binding to fibrin or fibrinogen.
32. The composition of any one of statements 25-31, wherein one or more of the antibodies, small molecules, polypeptides, or a combination thereof, are present the composition in an amount sufficient to reduce Mac-1 binding to fibrin or fibrinogen.
33. The composition of any one of statements 25-32, formulated in an amount sufficient to reduce SARS-COV-2 viral entry and/or SARS-COV-1 viral entry into cells.
34. The composition of any one of statements 25-33, formulated in an amount sufficient to reduce SARS-COV-2 viral entry into cells and/or SARS-COV-1 viral entry into cells by at least 25%, or at least 30%, or at least 40%, or at least 50%, or at least 60%, or at least 70%, or at least 80%, or at least 90%, or at least 95%, or at least 98%.
35. The composition of any one of statements 25-34, formulated in an amount sufficient to reduce fibrin deposition in tissues.
36. The composition of any one of statements 25-35, formulated in an amount sufficient to reduce fibrin deposition in tissues by at least 25%, or at least 30%, or at least 40%, or at least 50%, or at least 60%, or at least 70%, or at least 80%, or at least 90%, or at least 95%, or at least 98%.
37. An isolated human or humanized antibody that binds human fibrin or fibrinogen γC domain and that inhibits SARS-COV-2 spike protein binding to fibrin or fibrinogen γC domain.
38. isolated antibody of statement 37, wherein the antibody inhibits Mac-1 protein binding to fibrin or fibrinogen γC domain.
39. The isolated antibody of statement 37 or 38, wherein at least one of the antibody variable domains comprises a SEQ ID NO:6-8, 10, 11 or 12 amino acid sequence, or wherein the antibody variable domains comprise a combination of CDR regions with sequences comprising SEQ ID NO:6-8, 10, 11, and 12.
40. A method comprising contacting at least one test agent with fibrin, fibrinogen, SARS-COV-2 spike protein, or SARS-COV-1 spike protein, and detecting whether at least one of the test agents binds to the fibrin, fibrinogen, or spike protein, to thereby identify a useful binding agent.
41. The method of statement 40, wherein at least one of the test agents is a small molecule, polypeptide, or antibody.
42. The method of statement 40 or 41, further comprising detecting whether one or more of the test agents or the useful binding agents binds to at least one peptide with SEQ ID NO:2, Bβ119-129 (YLLKDLWQKRQ, SEQ ID NO:41), γ163-181 (QSGLYFIKPLKANQQFLVY; SEQ ID NO:42), γ364-395 (DNGIIWATWKTRWYSMKKTTMKIIPFNRLTIG; SEQ ID NO:43), or IIPFXRLXI (SEQ ID NO:64).
43. The method of any one of statements 40-42, further comprising determining whether one or more of the test agents or the useful binding agents will compete with the 5B8 antibody for binding to fibrin or fibrinogen.
44. The method of any one of statements 40-43, further comprising determining whether one or more of the test agents or the useful binding agents competes with fibrin for binding to the SARS-COV-2 spike protein or SARS-COV-1 spike protein.
45. The method of any one of statements 40-44, further comprising determining whether one or more of the test agents or the useful binding agents inhibits binding of Mac-1 with fibrin.
46. The method of any one of statements 40-45, further comprising determining whether one or more of the test agents or the useful binding agents will compete with the SARS-COV-2 spike protein or the SARS-COV-1 spike protein for binding to fibrin or fibrinogen.
47. The methods of any one of statements 40-46, further comprising quantifying the affinity and/or specificity of binding of the test agent or the or the useful binding agent to fibrin, fibrinogen, or spike protein.
48. The methods of any one of statements 40-47, further comprising administering the useful binding agent to an animal infected with SARS-COV-2 or SARS-Co-V-1 virus and determining whether the useful binding agent reduces inflammation or oxidative stress associated with the SARS-COV-2 or SARS-CoV-1 infection within the animal.
49. The method of statement 48, further comprising determining whether the useful binding agent reduces inflammation or oxidative in the brain, gut, kidneys, vascular system, or lungs of the animal infected with SARS-COV-2 or SARS-CoV-1 virus.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a nucleic acid” or “a protein” or “a cell” includes a plurality of such nucleic acids, proteins, or cells (for example, a solution or dried preparation of nucleic acids or expression cassettes, a solution of proteins, or a population of cells), and so forth. In this document, the term “or” is used to refer to a nonexclusive or, such that “A or B” includes “A but not B,” “B but not A,” and “A and B,” unless otherwise indicated

Under no circumstances may the patent be interpreted to be limited to the specific examples or embodiments or methods specifically disclosed herein Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

1. A method comprising administering a composition comprising anti-fibrin/anti-fibrinogen antibodies to a subject infected with SARS-COV-2 or SARS-COV-1.

2. The method of claim 1, which further comprises one or more antibodies that bind to a SARS-COV-2 spike protein or SARS-COV-1 spike protein.

3. The method of claim 1, which reduces SARS-COV-2 virus binding or SARS-CoV-2 Spike protein binding to fibrin or fibrinogen in the subject.

4. The method of claim 1, which reduces SARS-COV-1 virus binding or SARS-CoV-1 Spike protein binding to fibrin or fibrinogen in the subject.

5. The method of claim 1, which reduces inflammation, oxidative stress, fibrin deposition, or a combination thereof, in tissues of the subject.

6. The method of claim 1, which reduces inflammation, oxidative stress, fibrin deposition, or a combination thereof, in the subject's brain, gut, kidneys, vascular system, lungs, or a combination thereof.

7. The method of claim 1, which reduces inflammation, oxidative stress, fibrin deposition, or a combination thereof, in tissues of the subject, compared to a control subject who did not receive the composition.

8. The method of claim 1, which inhibits at least 50% of SARS-COV-2 spike protein, SARS-COV-1 spike protein, SARS-COV-2 viral particle, SARS-COV-1 viral particle, or Mac-1 binding to the fibrin or fibrinogen, compared to SARS-COV-2 spike protein, SARS-COV-1 spike protein, SARS-COV-2 viral particle, SARS-COV-1 viral particle, or Mac-1 binding to fibrin or fibrinogen in a control subject who did not receive the composition.

9. The method of claim 1, wherein SARS-COV-2 spike protein, SARS-COV-1 spike protein, SARS-COV-2 viral particle, SARS-COV-1 viral particle, or Mac-1 binding is to the fibrin γC domain or the fibrinogen γC domain.

10. The method of claim 1, wherein the antibodies are human antibodies or humanized antibodies.

11. The method of claim 1, wherein the anti-fibrin antibodies/anti-fibrinogen antibodies bind to at least one epitope with peptide sequence SEQ ID NO:2, Bβ119-129 (YLLKDLWQKRQ, SEQ ID NO:41), γ163-181 (QSGLYFIKPLKANQQFLVY; SEQ ID NO:42), γ364-395 (DNGIIWATWKTRWYSMKKTTMKIIPFNRLTIG; SEQ ID NO:43), IIPFXRLXI (SEQ ID NO:64), or a combination thereof.

12. The method of claim 1, wherein the anti-fibrin antibodies/anti-fibrinogen antibodies comprise a CDR region with a sequence comprising SEQ ID NO:6-8, 10-12, or combination of CDR regions with sequences comprising SEQ ID NO:6-8, 10, 11, and 12.

13. The method of claim 1, wherein the composition is administered at a time while SARS-COV-2 or SARS-COV-1 viral replication is occurring in the subject.

14. The method of claim 1, wherein the SARS-COV-2 is non-infectious SARS-CoV-2 or non-infectious SARS-COV-1.

15. The method of claim 1, wherein the composition is administered at a time after SARS-COV-2 or SARS-COV-1 viral replication in the subject is no longer replicating or is not detected.

16. A composition comprising one or more antibodies, small molecules, polypeptides, or a combination thereof in an amount sufficient to inhibit SARS-COV-2 spike protein or SARS-COV-1 spike protein binding fibrin or fibrinogen in a mammalian subject.

17. The composition of claim 16, wherein one or more of the antibodies are anti-fibrin antibodies/anti-fibrinogen antibodies.

18. The composition of claim 16, wherein one or more of the antibodies further comprise one or more antibodies that bind to a SARS-COV-2 spike protein or SARS-CoV-1 spike protein.

19. The composition of claim 16, which is formulated in an amount sufficient to reduce inflammation, oxidative stress, fibrin binding, or a combination thereof, in the subject's brain, gut, kidneys, vascular system, lungs, or a combination thereof, compared to a control subject who did not receive the composition.

20. The composition of claim 16, formulated in an amount that reduces SARS-CoV-2 virus and/or or SARS-COV-1 virus binding to fibrin or fibrinogen compared to SARS-COV-2 virus and/or SARS-COV-1 virus binding to fibrin or fibrinogen of a control subject who did not receive the composition.

21. The composition of claim 16, formulated in an amount that reduces Mac-1 protein binding to fibrin or fibrinogen compared to Mac-1 binding to fibrin or fibrinogen of a control subject who did not receive the composition.

22. The composition of claim 16, which inhibits at least 50% of SARS-COV-2 spike protein, SARS-COV-1 spike protein, SARS-COV-2 viral particle, SARS-CoV-1 viral particle, or Mac-1 binding to the fibrin or fibrinogen, compared to SARS-COV-2 spike protein, SARS-COV-1 spike protein, SARS-COV-2 viral particle, SARS-COV-1 viral particle, or Mac-1 binding to fibrin or fibrinogen in a control subject who did not receive the composition.

23. The composition of claim 22, wherein SARS-COV-2 spike protein, SARS-CoV-1 spike protein, SARS-COV-2 viral particle, SARS-COV-1 viral particle, or Mac-1 binding is to the fibrin γC domain or the fibrinogen γC domain.

24. The composition of claim 16, wherein the antibodies are human antibodies or humanized antibodies.

25. The composition of claim 17, wherein the anti-fibrin antibodies/anti-fibrinogen antibodies bind to at least one epitope with peptide sequence SEQ ID NO:2, Bβ119-129 (YLLKDLWQKRQ, SEQ ID NO:41), γ163-181 (QSGLYFIKPLKANQQFLVY; SEQ ID NO:42), γ364-395 (DNGIIWATWKTRWYSMKKTTMKIIPFNRLTIG; SEQ ID NO:43), IIPFXRLXI (SEQ ID NO:64), or bind to a combination thereof.

26. The composition of claim 17, wherein the anti-fibrin antibodies/anti-fibrinogen antibodies comprise a CDR region with a sequence comprising SEQ ID NO:6-8, 10-12, or combination of CDR regions with sequences comprising SEQ ID NO:6-8, 10, 11, and 12.

27. The composition of claim 16, wherein at least one antibody is an antibody fragment.

28. The composition of claim 16, wherein the SARS-COV-2 or SARS-Co-V-1 is infectious SARS-COV-2 or SARS-COV-1.

29. A method comprising (a) contacting at least one test agent with fibrin or fibrinogen and detecting whether at least one of the test agents binds to the fibrin or fibrinogen to thereby identify a binding agent; and/or (b) contacting the binding agent with a combination of fibrin and SARS-COV-2 Spike protein and detecting whether the binding agent inhibits binding of the SARS-COV-2 Spike protein to fibrin to thereby identify a useful binding agent.

30. The method of claim 29, wherein the test agent is a small molecule, polypeptide, or antibody.

31. (canceled)

32. (canceled)

33. (canceled)
